(19) United States
(12) Patent Application Publication
(10) Pub. No.: US 2018/0112217 A1
(43) Pub. Date: Apr. 26, 2018

(54) Stereospecific Phosphorothioate LNA

(71) Applicant: Roche Innovation Center Copenhagen A/S, Hørsholm (DK)

(72) Inventors: Henrik Frydenlund Hansen, Ringsted (DK); Troels Koch, Kobenhaven S. (DK); Nanna Albaek, Birkerød (DK); Jacob Ravn, Skovlunde (DK); Christoph Rosenbohm, Birkerød (DK); Peter Hagedorn, Hørsholm (DK)

(21) Appl. No.: 15/527,832

(22) PCT Filed: Nov. 18, 2015
(86) PCT No.: PCT/EP2015/076971

§ 371 Date: May 18, 2017

(30) Foreign Application Priority Data
Nov. 19, 2014 (EP) 14193887.8
Dec. 16, 2014 (EP) 14198167.0
Aug. 25, 2015 (EP) 15182401.8
Oct. 22, 2015 (EP) 15191074.2
Oct. 22, 2015 (EP) 15191075.9
Oct. 22, 2015 (EP) 15191076.7

(51) Int. Cl. C12N 15/117

(52) U.S. Cl. CPC C12N 15/117; C12N 2310/17; C12N 2310/315; C12N 2310/3231; C12N 2310/351; C12N 2310/341; C12N 2330/30; C12N 2320/53; C12N 2310/346

(57) ABSTRACT

The present invention provides stereodefined phosphorothioate LNA oligonucleotide, comprising at least one stereodefined phosphorothioate linkage between a LNA nucleoside and a subsequent (3′) nucleoside.

### FIELD OF INVENTION

The present invention provides stereodefined phosphorothioate LNA oligonucleotides, comprising at least one stereodefined phosphorothioate linkage between a LNA nucleoside and a subsequent (3′) nucleoside.

### BACKGROUND

Koziolkiewicz et al. (NAR 1995 24; 5000-5005) discloses 15mer DNA phosphorothioate oligonucleotides where the phosphorothioate linkages are either [all-Rp] configuration, or [all-Sp] configuration, or a random mixture of diastereomers. The [all-Rp] was found to be “more susceptible to” RNAaseH dependent degradation compared to the hybrids or [all-Rs] oligonucleotides, and was found to have a higher duplex thermal stability. It is suggested that for practical application, the [all-Rp] oligos should be protected by [Sp] phosphorothioates at their 3′ end.

Stec et al. (J. Am. Chem. Soc. 1998, 120; 7156-7167) reports on new monomers of 5′-=-DMT-deoxyribnucleoside 3′-O-(2-thio-“spiro”-4,4-pentamethylene-1,2,3-oxathiaphospholane) for use in sterocontrolled synthesis of PS-oligos via the oxathiaphospholane approach.

Karwowski et al. (Bioorganic & Med. Chem. Letts. 2001 11; 1001-1003) uses the oxathiaphospholane approach for the sterocontrolled synthesis of LNA dinucleoside phosphorothioates. The R steroisomer dinucleotide was readily hydrolysed by snake venom phosphodiesterase

Krieg et al. (Oligonucleotides 13; 491-499) investigated whether the immune stimulation by CpG PS-oligos depend on the chirality of their P-chirality. CpG PS Rp oligos showed much higher MAPK activation and induction of IκB degradation as compared to Sp oligos. There was no evidence for differential uptake of the different steroisomer oligonucleotides. The Rp oligonucleotides had a shorter duration (less than 48 hours), probably due to rapid degradation. For immune stimulation, CpG oligos with Rp chirality are suggested for rapid short term use, and the Sp oligos for longer term effect.

Levin et al. Chapter 7 Antisense Drug Technology 2008; 183-215 reviews phosphorotioate chirality, confirming that the chirality of phosphorothioate DNA oligonucleotides greatly effects their pharmacokinetics, not least due to the exonuclease resistance of the Sp stereoisomer. The PK effects of phosphorothioate chirality are reported to be less significant in second generation ASOs due to the 2′ modifications at the 3′ and 5′ termini which prevents exonuclease degradation, but it is likely that individual molecules which have Rp terminal residues may be more susceptible to exonucleases→i.e. for longer half-lives, the molecules with Sp residues at the termini are likely to have longer half-life.

Wave Life Sciences Poster (TIDES, May 3-6, 2014, San Diego): Based on the calculation of 524,288 possible different stereoisomers within mipomersen they illustrate 7 stereoisomers which differ markedly with respect to Tm, RNAseH recruitment, lipophilicity, metabolic stability, efficacy in vivo, and specific activity.

Wan et al, Nucleic Acids Research, Nov. 14, 2014 (advanced publication), discloses 31 antisense oligonucleotides where the chirality of the gap region was controlled using the DNA-oxazaphosphpoline approach (Oka et al., J. Am. Chem. Soc. 2008; 16031-16037.), and concluded that controlling PS chirality in the gap region of gapmers provides no significant benefits for therapeutic applications relative to the mixture of stereo-random PS ASOs. Wan et al. further refers to the added complexity and costs associated with the synthesis and characterization of chiral PS ASOs as minimizing their utility.

Swayze et al., 2007, NAR 35(2): 687-700 reports that LNA antisense compounds improve potency but cause significant hepatotoxicity in animals. WO 2008/049085 reports on LNA mixed wing gapmers which comprise 2′-O-MOE in the LNA flanking regions, which apparently reduce the toxicity of certain LNA compounds, but significantly reduce the potency.

WO2014/012081 and WO2014/010250 provide chiral reagents for synthesis of oligonucleotides.

WO2015/107425 reports on the chiral designs of chirally defined oligonucleotides, and reports that certain chirally defined compounds can alter the RNaseH cleavage pattern.

### SUMMARY OF INVENTION

The invention provides for a stereodefined phosphorothioate LNA oligonucleotide, comprising at least one stereodefined phosphorothioate linkage between a LNA nucleoside and a subsequent (3′) nucleoside. The term stereodefined is used interchangeably with the term stereoselective herein.

The invention provides for a stereodefined phosphorothioate LNA oligonucleotide of which comprises at least one stereospecific phosphorothioate nucleotide pair wherein the internucleoside linkage between the nucleosides of the stereodefined phosphorothioate nucleotide pair is either in the Sp configuration or in the Rp configuration, and wherein at least one of the nucleosides of the nucleotide pair is a LNA nucleoside.

In some embodiments, the LNA oligonucleotide of the invention is a gapmer oligonucleotide. The invention provides for a stereodefined phosphorothioate LNA oligonucleotide, comprising at least one stereodefined phosphorothioate linkage between a LNA nucleoside and a subsequent (3′) nucleoside; wherein the LNA oligonucleotide is a gapmer oligonucleotide.

In some embodiments of the LNA oligonucleotide of the invention, such as the gapmer oligonucleotide, the other nucleoside of the stereodefined phosphorothioate nucleotide pair is other than DNA, such as nucleoside analogue, such as a further LNA nucleoside or a 2′ substituted nucleoside.

In some embodiments of the LNA oligonucleotide of the invention, such as the gapmer oligonucleotide, the phosphorothioate internucleoside linkage between at least two adjacent LNA nucleosides is stereospecific, Sp or Rp.

In some embodiments of the LNA gapmer oligonucleotide of the invention, each wing of the gapmer comprises one or more stereospecific phosphorothioate internucleoside linkage between at least two adjacent LNA nucleosides.

In some embodiments of the LNA oligonucleotide of the invention, such as the gapmer oligonucleotide, all the phosphorothioate internucleoside linkages between adjacent LNA nucleosides are stereospecific.

The oligonucleotide of the invention is a LNA oligonucleotide, i.e. it comprises at least one LNA unit. In some embodiments of the LNA gapmer oligonucleotide of the invention, may further comprise other nucleoside units, such as DNA nucleosides. In some embodiments the oligonucleotide of the invention may further comprise at least one 2′ substituted nucleoside analogue units, such as, for example, 2′-O-methoxyethyl-RNA (2′MOE), 2′-fluoro-DNA units. In some embodiments the oligonucleotide of the invention comprises at least one LNA unit, at least one 2′ substituted nucleoside analogue unit, such as, for example at least one 2′-O-methoxyethyl-RNA (2′MOE) unit or at least one 2′-fluoro-DNA units, and at least one DNA unit.

In some embodiments of the LNA gapmer oligonucleotide of the invention, the oligonucleotide comprises a region Y′ which is capable of recruiting RNase H, which is flanked 5′ and 3′ by 1-6 nucleoside analogue units, such as LNA or 2′ substituted nucleoside analogue units.

In some embodiments, the nucleoside analogue units are independently selected from the group consisting of 2′-O-methoxyethyl-RNA (2′MOE), 2′-fluoro-DNA units (monomers) or LNA nucleoside units (monomers). Therefore in some embodiments, the oligonucleotide comprises both at least one LNA unit and at least one 2′ substituted nucleoside analogue unit, such as 2′-O-methoxyethyl-RNA (2′MOE) or 2′-fluoro-DNA units.

In some embodiments, the nucleoside analogue units present in the oligonucleotide of the invention, such as the gapmer oligonucleotide are LNA units.

In some embodiments, the LNA units in the stereodefined phosphorothioate LNA oligonucleotide comprise or are selected from the group consisting of (R)-cET, and (S)-cET.

In some embodiments, the LNA units in the stereodefined phosphorothioate LNA oligonucleotide comprise or are beta-D-oxy LNA units.

The invention further provides for a conjugate comprising the stereodefined phosphorothioate LNA oligonucleotide of the invention.

The invention further provides for a pharmaceutical composition comprising the stereodefined phosphorothioate LNA oligonucleotide of the invention and an a pharmaceutically acceptable solvent, (such as water or saline water), diluent, carrier, salt or adjuvant.

The invention further provides for a stereodefined phosphorothioate LNA oligonucleotide or conjugate of the invention, for use in medicine.

Pharmaceutical and other compositions comprising an oligomer of the invention are also provided. Further provided are methods of down-regulating the expression of a target nucleic acid, e.g. an RNA, such as a mRNA or microRNA in cells or tissues comprising contacting said cells or tissues, in vitro or in vivo, with an effective amount of one or more of the oligomers, conjugates or compositions of the invention.

Also disclosed are methods of treating an animal (a non-human animal or a human) suspected of having, or susceptible to, a disease or condition, associated with expression, or over-expression of a RNA by administering to the non-human animal or human a therapeutically or prophylactically effective amount of one or more of the oligomers, conjugates or pharmaceutical compositions of the invention.

The invention provides for methods of inhibiting (e.g., by down-regulating) the expression of a target nucleic acid in a cell or a tissue, the method comprising the step of contacting the cell or tissue, in vitro or in vivo, with an effective amount of one or more oligomers, conjugates, or pharmaceutical compositions thereof, to affect down-regulation of expression of a target nucleic acid.

The invention provides an LNA-gapmer oligonucleotide which comprises at least one stereodefined phosphorothioate internucleoside linkage within the gap region, wherein the LNA-gapmer comprises at least one beta-D-oxy LNA nucleoside unit.

The invention provides an LNA-gapmer oligonucleotide greater than 12 nucleotides in length, which comprises at least one stereodefined phosphorothioate internucleoside linkage within the gap region. In some embodiments the LNA-gapmer comprises at least one beta-D-oxy LNA nucleoside unit or at least one ScET nucleoside unit.

The invention provides for a phosphorothioate LNA oligonucleotide, comprising at least one stereodefined phosphorothioate linkage between a LNA nucleoside and a subsequent (3′) nucleoside. Such an LNA oligonucleotide may for example be a LNA gapmer, such as those as described or claimed herein. Such an oligonucleotide may be described as stereoselective.

In some embodiments, the LNA oligonucleotide of the invention comprises at least one stereodefined phosphorothioate linkage between a LNA nucleoside and a subsequent (3′) nucleoside. A stereodefined phosphorothioate linkage may also be referred to as a stereoselective or stereospecific phosphorothioate linkage.

In some embodiments, the LNA oligonucleotide of the invention comprises at least one stereodefined phosphorothioate nucleotide pair wherein the internucleoside linkage between the nucleosides of the stereodefined phosphorothioate nucleotide pair is either in the Rp configuration or in the Rs configuration, and wherein at least one of the nucleosides of the nucleotide pair is a LNA nucleotide. In some embodiments, the other nucleoside of the stereodefined phosphorothioate nucleotide pair is other than DNA, such as nucleoside analogue, such as a further LNA nucleoside or a 2′ substituted nucleoside.

The invention provides for a stereodefined phosphorothioate oligonucleotide which has a reduced toxicity in vivo or in vitro as compared to a non-stereodefined phosphorothioate oligonucleotide (parent) with the same nucleobase sequence and chemical modifications (other than the stereodefined phosphorothioate linkage(s)). In some embodiments, the non-stereodefined phosphorothioate oligonucleotide/stereodefined oligonucleotide may be a gapmer, such as a LNA-gapmer, or a mixmer a totolmer or one of the other oligonucleotide designs disclosed herein. For the comparison of toxicity, the stereodefined phosphorothioate oligonucleotide retains the pattern of modified and unmodified nucleosides present in the parent oligonucleotide

The invention provides for the use of a stereodefined phosphorothioate internucleoside linkage in an oligonucleotide, wherein the oligonucleotide has a reduced toxicity as compared to an identical oligonucleotide which does not comprise the sterospecified phosphorothioate internucleotide linkage.

The invention provides for the use of a stereocontrolling phosphoramidite monomer for the synthesis for a reduced toxicity oligonucleotide (a stereodefined phosphorothioate oligonucleotide).

The invention provides a method of altering the biodistribution of an antisense oligonucleotide sequence (parent oligonucleotide), comprising the steps of
a. Creating a library of stereodefined oligonucleotide variants (child oligonucleotides), retaining the core nucleobase sequence of the parent oligonucleotide,
b. Screening the library created in step a. for their biodistribution
c. Identify one or more stereodefined variants present in the library which has an altered (such as preferred) biodistribution as compared to the parent oligonucleotide.

It is recognised that in some embodiments, the parent oligonucleotide may be a mixture of different stereoisomeric forms, and as such the method of the invention may comprise a method of identifying individual stereodefined oligonucleotides, or individual stereoisomers (child oligonucleotides) which have one or more improved property, such as reduced toxicity, enhanced specificity, altered biodistribution, enhanced potency as compared to the patent oligonucleotide.

In some embodiments the compounds of the invention, or identified by the methods of the invention, have an enhanced biodistribution to the liver.

In some embodiments the compounds of the invention, or identified by the methods of the invention, have an enhanced liver/kidney biodistribution ratio.

In some embodiments the compounds of the invention, or identified by the methods of the invention, have an enhanced kidney/liver biodistribution ratio.

In some embodiments the compounds of the invention, or identified by the methods of the invention, have an enhanced biodistribution to the kidney.

In some embodiments the compounds of the invention, or identified by the methods of the invention, have an enhanced cellular uptake in hepatocytes.

In some embodiments the compounds of the invention, or identified by the methods of the invention, have an enhanced cellular uptake in kidney cells.

When referring to compounds with enhanced functional characteristics, the enhancement may be made with regards the parent oligonucleotide, such as an otherwise identical non-stereodefined oligonucleotide.

Whilst biodistribution studies are typically performed in vivo, they may also be performed in in vitro systems, by example by comparing the cellular uptake in different cell types, for examples in in vitro hepatotcytes (e.g. primary hepatocytes) or renal cells (e.g. renal epithelial cells, such as PTEC-TERT1 cells).

### FIGURES

FIG. 1: A schematic view of some LNA oligonucleotide of the invention. Regions X′ and Z′ comprise at least one stereodefined phosphorothioate internucleoside linkage between a LNA nucleoside and a 3′ nucleoside, and may for example all be LNA nucleosides with stereodefined phosphorothioate internucleoside linkages between them, and optionally between region X′ and Y′ and between region Y′ and Z′. The figure shows a 3-10-3 gapmer oligonucleotide with 15 internucleoside phosphorothioate linkages. The internucleoside linkages in the wing regions X′ and Y′ may be as described herein, for example may be randomly Rp or Sp phosphorothioate linkages. The table part of FIG. 1 provides a parent compound (P) where all the internucleoside linkages of the gap region Y′ are also randomly incorporated Rp or Sp phosphorothioate linkages (M), and in compounds 1-10, one of the phosphorothioate linkages is stereodefined as a Rp phosphorothioate internucleoside linkage (R).

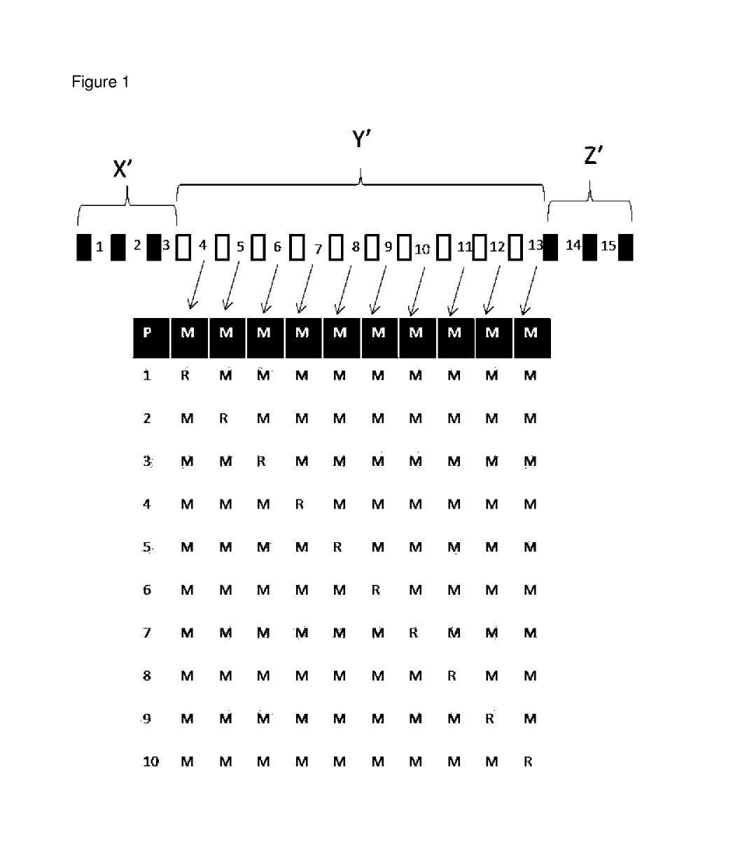

FIG. 2: As per FIG. 1, except in compounds 1-10, one of the phosphorothioate linkages is stereodefined as a Sp phosphorothioate internucleoside linkage (S).

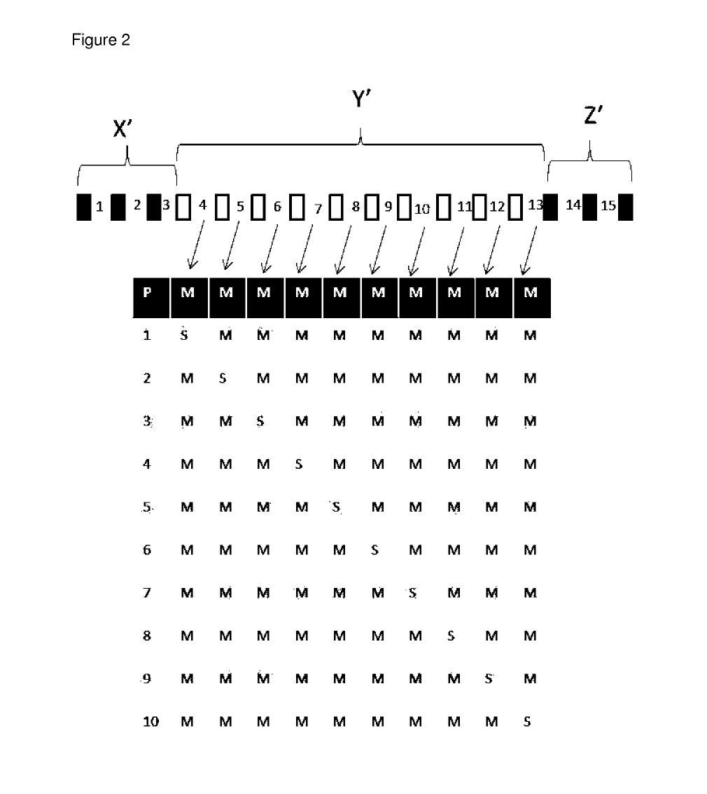

FIG. 3: The hepatotoxic potential (ALT) for LNA oligonucleotides where 3 phosphorothioate internucleoside linkages are fixed in either S (Comp #10) or R (Comp #14) configuration was compared to the ALT for parent mixture of diastereoisomers (Comp #1) with all internucleoside linkages as mixtures of R and S configuration.

FIG. 4: Oligonucleotide content in liver, kidney, and spleen

FIG. 5: Changes in LDH levels in the supernatants and intracellular ATP levels of cells treated for 3 days with the respective LNAs. Target knockdown (Myd88) was evaluated after 48 hours.

FIG. 6: In vitro toxicity screening in primary hepatocytes: Changes in LDH levels in the supernatants and intracellular ATP levels of cells treated for 3 days with the respective LNAs. Data are mean values and expressed as % vehicle control (n=4 experiments in triplicates for #56 and n=2 experiments in triplicates for all other LNAs).

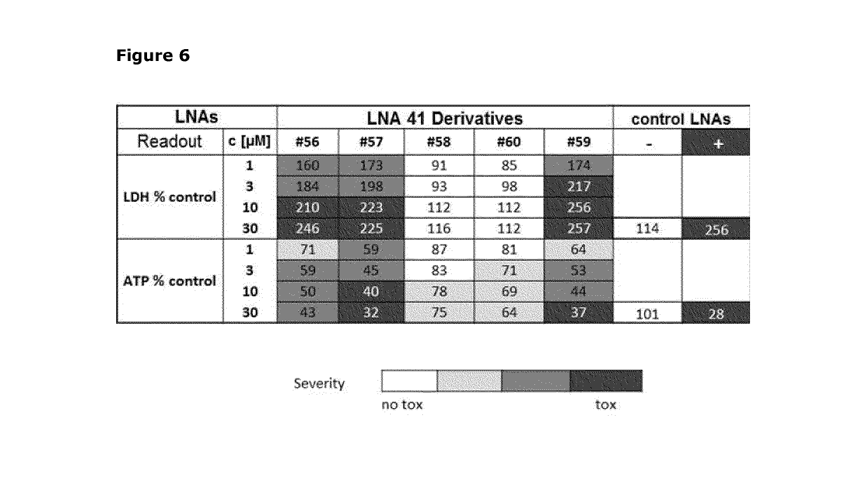

FIG. 7: In vitro toxicity screening in kidney proximal tubule cells: Viability of PTEC-TERT1 treated with LNA Myd88 stereovariants at 10 μM and 30 μM as measured after 9 days (cellular ATP).

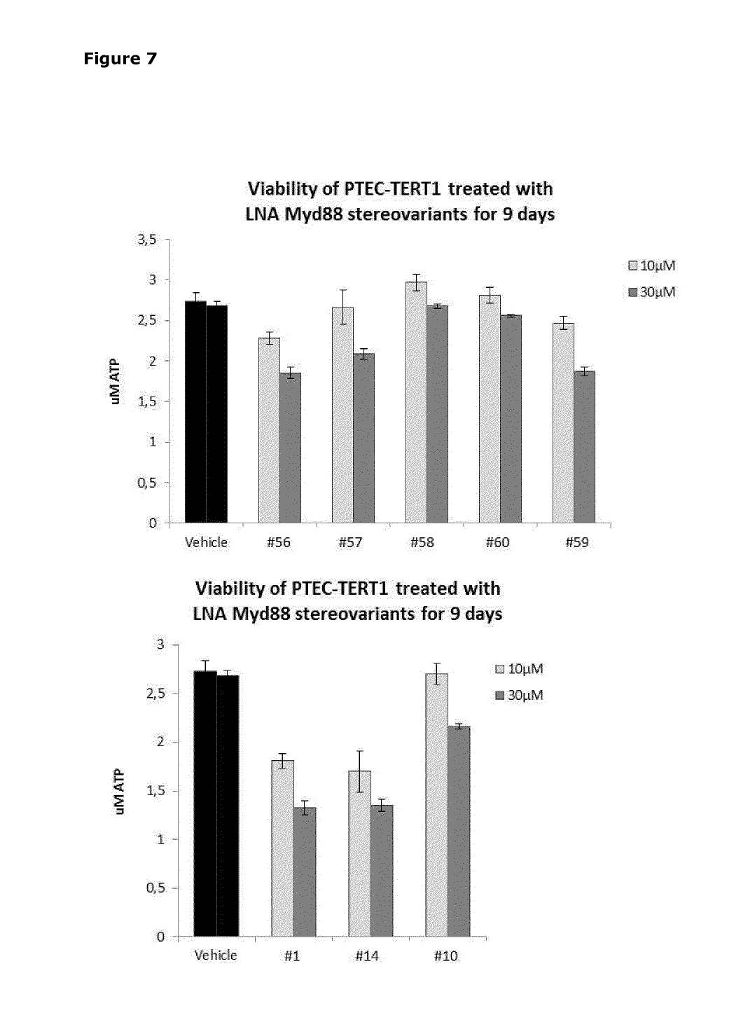

### DETAILED DESCRIPTION OF INVENTION

LNA Monomers

LNA monomers (also referred to as BNA) are nucleosides where there is a biradical between the 2′ and 4′ position of the ribose ring. The 2′-4′ biradical is also referred to as a bridge. LNA monomers, when incorporated into a oligonucleotides are known to enhance the binding affinity of the oligonucleotide to a complementary DNA or RNA sequence, typically measured or calculated as an increase in the temperature required to melt the oligonucleotide/target duplex (Tₘ).

The invention provides for LNA-oxazaphopholine monomers which may be used in methods of synthesis of oligonucleotides. For example, the LNA oxazaphopholine monomers may be as according to the formula 1A, 1B; 2A, 2B, 3A, 3B; 4A, 4B; 5A, 5B; 6A, 6B, or 7A-7H herein.

The Oligomer

The present invention employs LNA oligomeric compounds (also referred herein as LNA oligomers or LNA oligonucleotides) for use in modulating, such as inhibiting a target nucleic acid in a cell. Oligonucleotides which comprise at least one LNA nucleoside may be referred to as an LNA oligonucleotide or LNA oligomer herein. The term oligonucleotide and oligomer are used interchangeably herein.

An LNA oligomer comprises at least one “Locked Nucleic Acid” (LNA) nucleoside, such as a nucleoside which comprises a covalent bridge (also referred to a radical) between the 2′ and 4′ position (a 2′-4′ bridge). LNA nucleosides are also referred to as “bicyclic nucleosides”. The LNA oligomer is typically a single stranded antisense oligonucleotide.

In some embodiments the LNA oligomer comprises or is a gapmer. In some embodiments the LNA oligomer comprises or is a mixmer. In some embodiments the LNA oligomer comprises or is a totalmer.

In some embodiments, the nucleoside analogues present in the oligomer are all LNA, and the oligomer may, optionally further comprise RNA or DNA, such as DNA nucleosides (e.g. in a gapmer or mixmer).

In various embodiments, the compound of the invention does not comprise RNA (units). In some embodiments, the oligomer has a single contiguous sequence which is a linear molecule or is synthesized as a linear molecule. The oligomer may therefore be single stranded molecule. In some embodiments, the oligomer does not comprise short regions of, for example, at least 3, 4 or 5 contiguous nucleotides, which are complementary to equivalent regions within the same oligomer (i.e. duplexes). The oligomer, in some embodiments, may be not (essentially) double stranded. The oligomer is essentially not double stranded, such as is not a siRNA. In some embodiments, the oligomeric compound is not in the form of a duplex with a (substantially) complementary oligonucleotide—e.g. is not an siRNA.

The term “oligomer” in the context of the present invention, refers to a molecule formed by covalent linkage of two or more nucleotides (i.e. an oligonucleotide). Herein, a single nucleotide (unit) may also be referred to as a monomer or unit. In some embodiments, the terms “nucleoside”, “nucleotide”, “unit” and “monomer” are used interchangeably. It will be recognized that when referring to a sequence of nucleotides or monomers, what is referred to is the sequence of bases, such as A, T, G, C or U.

In some embodiments the oligonucleotide of the invention is 10-20 nucleotides in length, such as 10-16 nucleotides in length. In some embodiments the oligonucleotide of the invention is 12-20 or 12-24 nucleotides in length, such as 12-20 or 12-24 nucleotides in length.

In some embodiments, the invention provides a phosphorothioate LNA gapmer oligonucleotide, comprising at least one stereodefined phosphorothioate linkage between a LNA nucleoside and a subsequent (3′) nucleoside, wherein at least one of the internucleoside linkages of central region is stereodefined, and wherein the central region comprises both Rp and Sp internucleoside linkages; and optionally wherein at least one of the LNA or 2′ substituted nucleosides region (X′) or (Z′) is a beta-D-oxy LNA nucleoside.

In some embodiments the gapmer comprises a central region (Y′) of at least 5 or more contiguous nucleosides, and a 5′ wing region (X′) comprising of 1-6 LNA or 2′ substituted nucleosides and a 3′ wing region (Z′) comprising of LNA 1-6 or 2′ substituted nucleosides.

The gapmer oligonucleotide of the invention may comprise a central region (Y′) of at least 5 or more contiguous nucleosides capable of recruiting RNaseH, and a 5′ wing region (X′) comprising of 1-6 LNA nucleosides and a 3′ wing region (Z′) comprising of LNA 1-6 nucleosides, wherein at least one of the internucleoside linkages of central region are stereodefined, and wherein the central region comprises both Rp and Sp internucleoside linkages. Suitably region Y′ may have 6, 7, 8, 9, 10, 11 or 12 (or in some embodiments 13, 14, 15 or 16) contiguous nucleotides, such as DNA nucleotides, and the nucleotides of regions X′ and Z′ adjacent to region Y′ are LNA nucleotides. In some embodiments regions X′ and Z′ have 1-6 nucleotides at least one of which in each flank (X′ and Z′) are an LNA. In some embodiments all the nucleotides in region X′ and region Z′ are LNA nucleotides. In some embodiments the oligonucleotide of the invention comprises LNA and DNA nucleosides. In some embodiments, the oligonucleotide of the invention may be a mixed wing LNA gapmer where at least one of the LNA nucleosides in one of the wing regions (or at least one LNA in each wing) is replaced with a DNA nucleoside, or a 2′ substituted nucleoside, such as a 2′MOE nucleoside. In some embodiments the LNA gapmer does not comprise 2′ substituted nucleosides in the wing regions.

The internucleoside linkages between the nucleotides in the contiguous sequence of nucleotides of regions X′—Y′—Z′ may be all phosphorothioate internucleoside linkages. In some embodiments, the internucleoside linkages within region Y′ are all stereodefined phosphorothioate internucleoside linkages. In some embodiments, the internucleoside linkages within region X′ and Y′ are stereodefined phosphorothioate internucleoside linkages. In some embodiments the internucleoside linkages between region X′ and Y′ and between region Y′ and Z′ are stereodefined phosphorothioate internucleoside linkages. In some embodiments all the internucleoside linkages within the contiguous nucleosides of regions X′—Y′—Z′ are stereodefined phosphorothioate internucleoside linkages.

The introduction of at least one stereodefined phosphorothioate linkage adjacent to a LNA nucleoside, or in one or both wing regions (optionally including the introduction of at least one stereodefined phosphorothioate linkages in the gap region) may be used to modulate the biological profile of the oligonucleotide, for example it may modulate the toxicity profile.

In some embodiments, 2, 3, 4 or 5 of the phosphorothioate linkages in the gap region are stereodefined. In some embodiments the remaining internucleoside linkages in the gap region are not stereodefined: They exist as a (e.g. racemic) mixture of Rp and Sp in the population of oligonucleotide species. In some embodiments the remaining internucleoside linkage in the oligonucleotide are not stereodefined. In some embodiments all the internucleoside linkages in the gap region are stereodefined. The gap region (referred to as Y′) herein, is a region of nucleotides which is capable of recruiting RNaseH, and may for example be a region of at least 5 contiguous DNA nucleosides. In some embodiments all the internucleoside linkages in the gap and wing regions are stereodefined (i.e. within X′—Y′—Z′). In some embodiments all of the phosphorothioate internucleoside linkages in the oligonucleotide of the invention are stereodefined phosphorothioate internucleoside linkages. In some embodiments, all of the internucleoside linkages in the oligonucleotide of the invention are stereodefined phosphorothioate internucleoside linkages.

Typically, oligonucleotide phosphorothioates are synthesised as a random mixture of Rp and Sp phosphorothioate linkages (also referred to as a racemic mixture). In the present invention, gapmer phosphorothioate oligonucleotides are provided where at least one of the phosphorothioate linkages of the gap region oligonucleotide is stereodefined, i.e. is either Rp or Sp in at least 75%, such as at least 80%, or at least 85%, or at least 90% or at least 95%, or at least 97%, such as at least 98%, such as at least 99%, or (essentially) all of the oligonucleotide molecules present in the oligonucleotide sample. Such oligonucleotides may be referred as being stereodefined, stereoselective or stereospecified: They comprise at least one phosphorothioate linkage which is stereospecific. The terms stereodefined and stereospecified/stereoselective may be used interchangeably herein. The terms stereodefined, stereoselective and stereospecified may be used to describe a phosphorothioate internucleoside linkage (Rp or Sp), or may be used to described a oligonucleotide which comprises such a phosphorothioate internucleoside linkage. It is recognised that a stereodefined oligonucleotide may comprise a small amount of the alternative stereoisomer at any one position, for example Wan et al reports a 98% stereoselectivity for the gapmers reported in NAR, November 2014.

In some embodiments 1, 2, 3, 4, 5, 6, 7, 8, 9, 10, 11, 12, 13, 14, or 15, of the linkages in the gap region of the oligomer are stereodefined phosphorothioate linkages.

In some embodiments 5%, 10%, 15%, 20%, 25%, 30%, 35%, 40%, 45%, 50%, 55%, 60%, 65%, 70%, 75%, 80%, 85%, 90%, or 95% of the linkages in the oligomer are stereodefined phosphorothioate linkages. In some embodiments all of the phosphorothioate linkages in the oligomer are stereodefined phosphorothioate linkages. In some embodiments the all the internucleoside linkages of the oligomer are stereodefined phosphorothioate linkages. It should be recognised that stereodefined (stereospecificity) refers to the incorporation of a high proportion, i.e. at least 75%, of either the Rp or the Sp internucleoside linkage at a defined internucleoside linkage.

LNA monomers (also referred to as bicyclic nucleic acids, BNA) are nucleosides where there is a biradical between the 2′ and 4′ position of the ribose ring. The 2′-4′ biradical is also referred to as a bridge. LNA monomers, when incorporated into a oligonucleotides are known to enhance the binding affinity of the oligonucleotide to a complementary DNA or RNA sequence, typically measured or calculated as an increase in the temperature required to melt the oligonucleotide/target duplex (Tₘ).

An LNA oligomer comprises at least one “Locked Nucleic Acid” (LNA) nucleoside, such as a nucleoside which comprises a covalent bridge (also referred to a radical) between the 2′ and 4′ position (a 2′-4′ bridge). LNA nucleosides are also referred to as “bicyclic nucleosides”. The LNA oligomer is typically a single stranded antisense oligonucleotide.

In some embodiments the LNA oligomer comprises or is a gapmer. In some embodiments, the nucleoside analogues present in the oligomer are all LNA.

In various embodiments, the compound of the invention does not comprise RNA (units). In some embodiments, the oligomer has a single contiguous sequence which is a linear molecule or is synthesized as a linear molecule. The oligomer may therefore be single stranded molecule. In some embodiments, the oligomer does not comprise short regions of, for example, at least 3, 4 or 5 contiguous nucleotides, which are complementary to equivalent regions within the same oligomer (i.e. duplexes). The oligomer, in some embodiments, may be not (essentially) double stranded. The oligomer is essentially not double stranded, such as is not a siRNA. In some embodiments, the oligomeric compound is not in the form of a duplex with a (substantially) complementary oligonucleotide—e.g. is not an siRNA.

The term “oligomer” in the context of the present invention, refers to a molecule formed by covalent linkage of two or more nucleotides (i.e. an oligonucleotide). Herein, a single nucleotide (unit) may also be referred to as a monomer or unit. In some embodiments, the terms “nucleoside”, “nucleotide”, “unit” and “monomer” are used interchangeably. It will be recognized that when referring to a sequence of nucleotides or monomers, what is referred to is the sequence of bases, such as A, T, G, C or U. The term monomer is used herein both to describe each unit of an oligonucleotide (nucleoside/nucleotide) as well as the (phosphoramidite) monomers used in oligonucleotide synthesis.

### Advantages

Traditional discovery of oligonucleotides for therapeutic application involve the screening of a large number of compounds across a large section or even the entire nucleic acid target—referred to as a gene walk. Whilst such an approach is useful in identifying accessible target sites in a mRNA, it results in compounds which are selected based on their in vitro hybridisation properties.

The present invention provides a method for optimising oligonucleotides, such as oligonucleotides identified by gene-walk for in vivo (e.g. pharmacological) utility. In particular the monomers of the present invention may be used in the synthesis of oligomers to enhance beneficial in vivo properties, such as serum protein binding, biodistribution, intracellular uptake, or to reduce undesirable properties, such as toxicity or inflammatory sensitivities. The monomers of the present invention may be used to reduce hepatotoxicity of LNA oligonucleotides in vivo. LNA hepatotoxicity may be determined using a model mouse system, see for example EP 1 984 381. The monomers of the present invention may be used to reduce nephrotoxicity of LNA oligonucleotides. LNA nephrotoxicity may be determined using a model rat system, and is often determined by the use of the Kim-1 biomarker (see e.g. WO 2014118267). The monomers of the present invention may be used to reduce the immunogenicity of an LNA oligomer in vivo. According to EP 1 984 381, LNAs with a 4′-CH₂—O-2′ radicals are particularly toxic.

The oligonucleotides of the invention may have improved nuclease resistance, biostability, target affinity, RNaseH activity, and/or lipophilicity. As such the invention provides methods for both enhancing the activity of the oligomer in vivo and improvement of the pharmacological and/or toxicological profile of the oligomer.

### Advantages

### RNaseH Recruitment

As illustrated in the examples, in some embodiments, the stereodefined oligonucleotides of the invention have an enhanced RNaseH recruitment activity as compared to an otherwise non-stereodefined oligonucleotide (the parent oligonucleotide). Indeed, the present inventors were surprised to find that in general, the introduction of stereodefined phosphorothioate internucleoside linkages into a RNaseH recruiting LNA oligonucleotide, e.g. a LNA gapmer oligonucleotide, resulted in an enhanced RNaseH recruitment activity, upto 30× that of the parent (non-stereodefined). The invention therefore provides for the use of a stereocontrolled (also referred to as stereospecific) phosphoramidite monomer for the synthesis for an oligonucleotide with enhanced RNaseH recruitment activity as compared to an otherwise identical non-stereodefined oligonucleotide.

The invention provides for a method for enhancing the RNaseH recruitment activity of an antisense oligonucleotide sequence (parent oligonucleotide) for a RNA target, comprising the steps of:

a. Creating a library of stereodefined oligonucleotide variants (child oligonucleotides), retaining the core nucleobase sequence of the parent oligonucleotide

b. Screening the library created in step a. for their in vitro RNaseH recruitment activity against a RNA target,

c. Identify one or more stereodefined variants present in the library which has an enhanced RNaseH recruitment activity as compared to the parent oligonucleotide.

d. Optionally manufacturing at least one of the stereodefined variants identified in step c.

The invention provides for an LNA oligonucleotide which has an enhanced RNaseH recruitment activity as compared to an otherwise identical non-stereodefined LNA oligonucleotide (or a parent oligonucleotide).

An otherwise identical non-stereodefined LNA oligonucleotide (e.g. a parent oligonucleotide) is a non-stereodefined phosphorothioate oligonucleotide with the same nucleobase sequence and chemical modifications, other than the stereodefined phosphorothioate linkage(s). It will be recognised that a non-stereodefined LNA oligonucleotide may comprise stereodefined centres in parts of the compound other than the phosphorothioate internucleotide linkages, e.g. within the nucleosides.

The use of chirally defined phosphorothioate linkages in LNA oligonucleotides surprisingly results in an increase in RNaseH activity. This may be seen when the gap-region comprises both stereodefined Rp and Sp internucleoside linkages. In some embodiments, the gap-region of the oligonucleotide of the invention comprises at least 2 Rp and at least 2 Sp stereodefined internucleoside linkages. In some embodiments the proportion of Rp vs. Sp stereodefined internucleoside linkages within gap region thereof (including internucleoside linkages adjacent to the wing regions), is between about 0.25 and about 0.75. In some embodiments, the gap-region of the oligonucleotide of the invention comprises at least 2 consecutive internucleoside linkages which are either stereodefined Rp or Sp internucleoside linkages. In some embodiments, the gap-region of the oligonucleotide of the invention comprises at least 3 consecutive internucleoside linkages which are either stereodefined Rp or Sp internucleoside linkages.

In some embodiments, the LNA oligonucleotide has an enhanced human RNaseH recruitment activity as compared to an equivalent non stereoselective LNA oligonucleotide, for example using the RNaseH recruitment assays provided in example 7. In some embodiments, the increase in RNaseH activity is at least 2×, such as at least 5×, such as at least 10× the RNaseH activity of the equivalent non stereoselective LNA oligonucleotide (e.g. parent oligonucleotide). Example 7 provides a suitable RNaseH assay which may be used to assess RNaseH activity (also referred to as RNaseH recruitment).

It has been found that a marked improvement in activity of RNaseH activity is found with LNA gapmer compounds where the gap region comprises both Rp and Sp internucleoside linkages, and in some embodiments, the gap region may comprise at least two Rp internucleoside linkages and at least two Sp internucleoside linkages, such as at least three Rp internucleoside linkages and/or at least three Sp internucleoside linkages.

It has been found that a marked improvement in activity of RNaseH activity is found with LNA gapmer compounds where the internucleoside linkages of the gap region are stereodefined. In some embodiments, therefore, there is at least one stereoselective phosphorothioate LNA oligonucleotide, comprising at least one stereoselective phosphorothioate linkage between a LNA nucleoside and a subsequent (3′) nucleoside. In some embodiments at least one of the internucleotide linkages within region X′ and/or Z′ is is a Rp internucleoside linkage. In some embodiments, the 5′ most internucleoside linkage in the oligomer or in region X′ is a Sp internucleoside linkage. In some embodiments the flanking regions X′ and Z′ comprise at least one Sp internucleoside linkage and at least one Rp internucleoside linkage. In some embodiments the 3′ internucleoside linkage of the oligomer or of region Z′ is a Sp internucleoside linkage.

In some embodiments, the stereodefined oligonucleotide of the invention has improved potency as compared to an otherwise non-stereodefined oligonucleotide or parent oligonucleotide.

### Specificity and Mismatch Discrimination

As illustrated in the examples, in some embodiments, the stereodefined oligonucleotides of the invention may have an enhanced mismatch discrimination (or enhanced target specificity) as compared to an otherwise non-stereodefined oligonucleotide (or parent oligonucleotide). Indeed, the present inventors were surprised to find that the introduction of stereodefined phosphorothioate internucleoside linkages into a RNaseH recruiting LNA oligonucleotide, e.g. a LNA gapmer oligonucleotide, may result in an enhanced mismatch discrimination (or target specificity). The invention therefore provides for the use of a stereocontrolling phosphorothioate monomer for the synthesis for an oligonucleotide with enhanced mismatch discrimination (or target specificity) as compared to an otherwise identical non-stereodefined oligonucleotide.

The invention therefore provides for method of enhancing the mismatch discrimination (or target specificity) of an antisense oligonucleotide sequence (parent oligonucleotide) for a RNA target in a cell, comprising the steps of

a. Creating a library of stereodefined oligonucleotide variants (child oligonucleotides), retaining the core nucleobase sequence of the parent oligonucleotide

b. Screening the library created in step a. for their activity against the RNA target and their activity for at least one other RNA present,

c. Identify one or more stereodefined variants present in the library which has a reduced activity against the at least one other RNA as compared to parent oligonucleotide. The reduced activity against the at least one other RNA may be determined as a ratio of activity of the intended target/unintended target (at least one other RNA). This method may be combined with the method for enhancing the RNaseH recruitment activity of an antisense oligonucleotide sequence (parent oligonucleotide) for a RNA target, to identify oligonucleotides of the invention which have both enhanced RNaseH recruitment activity and enhanced mismatch discrimination (i.e. enhanced targeted specificity).

The invention provides for an LNA oligonucleotide which has an enhanced mismatch discrimination (or enhanced target specificity) as compared to an otherwise identical non-stereodefined LNA oligonucleotide (or a parent oligonucleotide).

The invention provides for an LNA oligonucleotide which has an enhanced RNaseH recruitment activity and an enhanced mismatch discrimination (or enhanced target specificity) as compared to an otherwise identical non-stereodefined LNA oligonucleotide (or a parent oligonucleotide).

The invention therefore provides for the use of a stereocontrolling/stereocontrolled (can also be referred to as a stereodefined or stereospecific) phosphoramidite monomer for the synthesis for an oligonucleotide with enhanced mismatch discrimination (or target specificity) and enhanced RNAseH recruitment activity as compared to an otherwise identical non-stereodefined oligonucleotide.

In some embodiments the stereocontrolling phosphoramidite monomer is a LNA stereospecific phosphoramidite monomer. In some embodiments the stereocontrolling phosphoramidite monomer is a DNA stereocontrolling phosphoramidite monomer. In some embodiments the stereospecific phosphoramidite monomer is a 2′modified stereospecific phosphoramidite monomer, such as a 2′methoxyethyl stereospecific phosphoramidite RNA monomer. Stereospecific phosphoramidite monomers may, in some embodiments, be oxazaphospholine monomers, such as DNA-oxazaphospholine LNA-oxazaphospholine monomers. In some embodiments, the stereospecific phosphoramidite monomers may comprise a nucleobase selected from the group consisting of A, T, U, C, 5-methyl-C or G nucleobase.

### In Vivo Optimisation

The present invention provides a method for optimising oligonucleotides, such as oligonucleotides identified by gene-walk for in vivo (e.g. pharmacological) utility. In particular the monomers of the present invention may be used in the synthesis of oligomers to enhance beneficial in vivo properties, such as serum protein binding, biodistribution, intracellular uptake, or to reduce undesirable properties, such as toxicity or inflammatory sensitivities.

### Reduced Toxicity

The invention provides a method of reducing the toxicity of an antisense oligonucleotide sequence (parent oligonucleotide), comprising the steps of
a. Creating a library of stereodefined oligonucleotide variants (child oligonucleotides), retaining the core nucleobase sequence of the parent oligonucleotide,
b. Screening the library created in step a. for their in vitro or in vivo toxicity in a cell,
c. Identify one or more stereodefined variants present in the library which has a reduced toxicity in the cell as compared to the parent oligonucleotide.

In some embodiments the reduced toxicity is reduced hepatotoxicity. Hepatotoxicity of an oligonucleotide may be assess in vivo, for example in a mouse. In vivo hepatotoxicity assays are typically based on determination of blood serum markers for liver damage, such as ALT, AST or GGT. Levels of more than three times upper limit of normal are considered to be indicative of in vivo toxicity. In vivo toxicity may be evaluated in mice using, for example, a single 30 mg/kg dose of oligonucleotide, with toxicity evaluation 7 days later (7 day in vivo toxicity assay).

Suitable markers for cellular toxicity include elevated LDH, or a decrease in cellular ATP, and these markers may be used to determine cellular toxicity in vitro, for example using primary cells or cell cultures. For determination of hepatotoxicity, mouse or rat hepatocytes may be used, including primary hepatocytes. Primary primate such as human hepatocytes may be used if available. In mouse hepatocytes an elevation of LDH is indicative of toxicity. A reduction of cellular ATP is indicative of toxicity. In some embodiments the oligonucleotides of the invention have a reduced in vitro hepatotoxicity, as determined in primary mouse hepatocyte cells, e.g. using the assay provided in Example 8.

In some embodiments the reduced toxicity is reduced nephrotoxicity. Nephrotoxicity may be assessed in vivo, by the use of kidney damage markers including a rise in blood serum creatinine levels, or elevation of kim-1 mRNA/protein. Suitably mice or rodents may be used.

In vitro kidney injury assays may be used to measure nephrotoxicity, and may include the elevation of kim-1 mRNA/protein, or changes in cellular ATP (decrease). In some embodiments, PTEC-TERT1 cells may be used to determine nephrotoxicity in vitro, for example by measuring cellular ATP levels. In some embodiments the oligonucleotides of the invention have a reduced in vitro nephrotoxicity, as determined in PTEC-TERT1 cells, e.g. using the assay provided in Example 9.

Other in vitro toxicity assays which may be used to assess toxicity include caspase assays, and cell viability assays, e.g. MTS assays. In some embodiments the reduced toxicity oligonucleotide of the invention comprises at least one stereodefined Rp internucleotide linkage, such as at least 2, 3, or 4 stereodefined Rp internucleotide linkage. The examples illustrate compounds which comprise stereodefined Rp internucleotide linkages that have a reduced hepatotoxicity in vitro and in vivo. In some embodiments, the at least one stereodefined Rp internucleotide linkage is present within the gap-region of a LNA gapmer.

In some embodiments the reduced toxicity oligonucleotide of the invention comprises at least one stereodefined Sp internucleotide linkage, such as at least 2, 3, or 4 stereodefined Sp internucleotide linkage. The examples illustrate compounds which have a reduced nephrotoxicity which comprise at least one stereodefined Sp internucleoside linkage. In some embodiments, the at least one stereodefined Sp internucleotide linkage is present within the gap-region of a LNA gapmer.

The invention provides for the use of a stereocontrolled (may also be referred to as stereospecific, or stereospecifying) phosphoramidite monomer for the synthesis for a reduced toxicity oligonucleotide, e.g. reduced hepatotoxicity or reduced nephrotoxicity oligonucleotide. In some embodiments the stereocontrolled phosphoramidite monomer is a LNA stereocontrolled phosphoramidite monomer. In some embodiments the stereocontrolled phosphoramidite monomer is a DNA stereocontrolled phosphoramidite monomer. In some embodiments the stereocontrolled phosphoramidite monomer is a 2′modified stereocontrolled phosphoramidite monomer, such as a 2′methoxyethyl stereocontrolled phosphoramidite RNA monomer. Stereocontrolled phosphoramidite monomers may, in some embodiments, be oxazaphospholine monomers, such as DNA-oxazaphospholine LNA-oxazaphospholine monomers.

The monomers of the present invention may be used to reduce hepatotoxicity of LNA oligonucleotides in vitro or in vivo.

LNA hepatotoxicity may be determined using a model mouse system, see for example EP 1 984 381. The monomers of the present invention may be used to reduce nephrotoxicity of LNA oligonucleotides. LNA nephrotoxicity may be determined using a model rat system, and is often determined by the use of the Kim-1 biomarker (see e.g. WO 2014118267). The monomers of the present invention may be used to reduce the immunogenicity of an LNA oligomer in vivo. According to EP 1 984 381, LNAs with a 4′-CH₂—O-2′ radicals are particularly toxic.

The oligonucleotides of the invention may have improved nuclease resistance, biostability, target affinity, RNaseH activity, and/or lipophilicity. As such the invention provides methods for both enhancing the activity of the oligomer in vivo and improvement of the pharmacological and/or toxicological profile of the oligomer.

In some embodiments, the LNA oligonucleotide has reduced toxicity as compared to an equivalent non stereoselective LNA oligonucleotide, e.g. reduced in vivo hepatotoxicity, for example as measured using the assay provided in example 6, or reduced in vitro hepatotoxicity, for example as measured using the assay provided in example 8, or reduced nephrotoxicity, for example as measured using the assay provided in example 9. Reduced toxicity may also be assessed using other methods known in the art, for example caspase assays and primary hepatocyte toxicity assays (e.g. example 8).

### The Target

The target of an oligonucleotide is typically a nucleic acid to which the oligonucleotide is capable of hybridising under physiological conditions. The target nucleic acid may be, for example a mRNA or a microRNA (encompassed by the term target gene). Such as oligonucleotide is referred to as an antisense oligonucleotide.

Suitably the oligomer of the invention is capable of down-regulating (e.g. reducing or removing) expression of the a target gene. In this regards, the oligomer of the invention can affect the inhibition of the target gene, typically in a mammalian such as a human cell. In some embodiments, the oligomers of the invention bind to the target nucleic acid and affect inhibition of expression of at least 10% or 20% compared to the normal expression level, more preferably at least a 30%, 40%, 50%, 60%, 70%, 80%, 90% or 95% inhibition compared to the normal expression level (such as the expression level in the absence of the oligomer(s) or conjugate(s)). In some embodiments, such modulation is seen when using from 0.04 and 25 nM, such as from 0.8 and 20 nM concentration of the compound of the invention. In the same or a different embodiment, the inhibition of expression is less than 100%, such as less than 98% inhibition, less than 95% inhibition, less than 90% inhibition, less than 80% inhibition, such as less than 70% inhibition. Modulation of expression level may be determined by measuring protein levels, e.g. by the methods such as SDS-PAGE followed by western blotting using suitable antibodies raised against the target protein. Alternatively, modulation of expression levels can be determined by measuring levels of mRNA, e.g. by northern blotting or quantitative RT-PCR. When measuring via mRNA levels, the level of down-regulation when using an appropriate dosage, such as from 0.04 and 25 nM, such as from 0.8 and 20 nM concentration, is, In some embodiments, typically to a level of from 10-20% the normal levels in the absence of the compound, conjugate or composition of the invention.

The invention therefore provides a method of down-regulating or inhibiting the expression of a target protein and/or target RNA in a cell which is expressing the target protein and/or RNA, said method comprising administering the oligomer or conjugate according to the invention to said cell to down-regulating or inhibiting the expression of the target protein or RNA in said cell. Suitably the cell is a mammalian cell such as a human cell. The administration may occur, in some embodiments, in vitro. The administration may occur, in some embodiments, in vivo.

The oligomers may comprise or consist of a contiguous nucleotide sequence which corresponds to the reverse complement of a nucleotide sequence present in the target nucleic acid.

In determining the degree of “complementarity” between oligomers of the invention (or regions thereof) and the target region of the nucleic acid the degree of “complementarity” (also, “homology” or “identity”) is expressed as the percentage identity (or percentage homology) between the sequence of the oligomer (or region thereof) and the sequence of the target region (or the reverse complement of the target region) that best aligns therewith. The percentage is calculated by counting the number of aligned bases that are identical between the 2 sequences, dividing by the total number of contiguous monomers in the oligomer, and multiplying by 100. In such a comparison, if gaps exist, it is preferable that such gaps are merely mismatches rather than areas where the number of monomers within the gap differs between the oligomer of the invention and the target region.

As used herein, the terms “homologous” and “homology” are interchangeable with the terms “identity” and “identical”.

The terms “corresponding to” and “corresponds to” refer to the comparison between the nucleotide sequence of the oligomer (i.e. the nucleobase or base sequence) or contiguous nucleotide sequence (a first region) and the equivalent contiguous nucleotide sequence of a further sequence selected from either i) a sub-sequence of the reverse complement of the nucleic acid target, such as the mRNA which encodes the target protein. WO2014/118267 provides numerous target mRNAs which are of therapeutic relevance, as well as oligomer sequences which may be optimised using the present invention (see e.g. table 1, the NCBI Genbank references are as disclosed in WO2014/118257)

TABLE 1

| The compound of the | |
|---|---|
| invention may target | |
| a nucleic acid (e.g. mRNA | |
| encoding, or miRNA) selected | For the treatment of a disease or |
| from the groups consisting of | disorder such as |
| AAT | AAT-LivD |
| ALDH2 | Alcohol dependence |
| HAMP pathway | Anemia or inflammation/CKD |
| Apo(a) | Atherosclerosis/high Lp(a) |
| Myc | Liver cancer |
| 5′UTR | HCV |
| 5′UTR & NS5B | HCV |
| NS3 | HCV |
| TMPRSS6 | Hemochromatosis |
| Antithrombin III | Hemophilia A, B |
| ApoCIII | Hypertriglyceridemia |
| ANGPLT3 | Hyperlipidaemia |
| MTP | Hyperlipidaemia |
| DGAT2 | NASH |
| ALAS1 | Porphyria |
| Antithrombin III | Rare Bleeding disorders |
| Serum amyloid A | SAA-amyloidosis |
| Factor VII | Thrombosis |
| Growth hormone receptor | Acromegaly |
| ApoB-100 | Hypercholesterolemia |
| ApoCIII | Hypertriglyceridemia |
| PCSK9 | Hypercholesterolemia |
| CRP | Inflammatory disorders |
| KSP or VEGF | Liver cancer |
| PLK1 | Liver cancer |
| FGFR4 | Obesity |
| Factor IXa | Thrombosis |
| Factor XI | Thrombosis |
| TTR | TTR amyloidosis |
| GCCR | Type 2 diabetes |
| PTP-1B | Type 2 diabetes |
| GCGR | Cushing's Syndrome |
| Hepatic Glucose 6-Phosphate | glucose homeostasis, diabetes, type 2 |
| Transporter-1 | diabetes |

In one embodiment, the target is selected from the group consisting of Myd88, ApoB, and PTEN.

The terms “corresponding nucleotide analogue” and “corresponding nucleotide” are intended to indicate that the nucleotide in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine.

The terms “reverse complement”, “reverse complementary” and “reverse complementarity” as used herein are interchangeable with the terms “complement”, “complementary” and “complementarity”.

### Length

The oligomer may consists or comprises of a contiguous nucleotide sequence of from 7-30, such as 7-26 or 8-25, such as 9, 10, 11, 12, 13, 14, 15, 16, 17, 18, 19, 20, 21, 22, 23, 24, 25 nucleotides in length, such as 10-20 nucleotides in length. In some embodiments, the length of the LNA oligomer is 10-16 nucleotides, such as 12, 13 or 14 nucleosides. In some embodiments, the LNA oligomer is 7, 8, 9 nucleosides in length, such as a “Tiny” LNA.

In some embodiments, the oligomers comprise or consist of a contiguous nucleotide sequence of a total of from 10-22, such as 12-18, such as 13-17 or 12-16, such as 13, 14, 15, 16 contiguous nucleotides in length.

In some embodiments, the oligomers comprise or consist of a contiguous nucleotide sequence of a total of 10, 11, 12, 13, or 14 contiguous nucleotides in length.

In some embodiments, the oligomer according to the invention consists of no more than 22 nucleotides, such as no more than 20 nucleotides, such as no more than 18 nucleotides, such as 15, 16 or 17 nucleotides. In some embodiments the oligomer of the invention comprises less than 20 nucleotides. It should be understood that when a range is given for an oligomer, or contiguous nucleotide sequence length it includes the lower an upper lengths provided in the range, for example from (or between) 10-30, includes both 10 and 30.

In some embodiments, LNA oligomers has a length of less than 20, such as less than 18, such as 16 nts or less or 15 or 14 nts or less. In some embodiments, the oligomers comprise or consist of a contiguous nucleotide sequence of a total of 10, 11, 12, 13, or 14 contiguous nucleotides in length.

In some embodiments, the oligomer according to the invention consists of no more than 22 nucleotides, such as no more than 20 nucleotides, such as no more than 18 nucleotides, such as 15, 16 or 17 nucleotides. In some embodiments the oligomer of the invention comprises less than 20 nucleotides. It should be understood that when a range is given for an oligomer, or contiguous nucleotide sequence length it includes the lower an upper lengths provided in the range, for example from (or between) 10-30, includes both 10 and 30.

### Stereo-Selective LNA Motifs

As referred to above, the invention provides for an oligonucleotide comprising at least one nucleotide pair wherein the internucleoside linkage between the nucleotides pair is either in the Rp configuration or in the Rs configuration, and wherein at least one of the nucleosides of the nucleotide pair is a LNA nucleotide. Such as nucleotide pair is referred to as a “LNA dinucleotide” herein. LNA dinucleotides may also be referred to as a stereospecific phosphorothioate LNA dinucleotide.

In some embodiments the oligonucleotide of the invention comprises more than one LNA dinucleotide, such as 2, 3, 4, 5, 6, 7, 8, 9 or 10 LNA dinucleotides.

In some embodiments the oligomer comprises a 5′ terminal LNA dinucleotide. In some embodiments the oligomer comprises a 3′ terminal LNA dinucleotide. In some embodiments the oligomer comprises both a 5′ and a 3′ terminal LNA dinucleotide, the stereospecificity of the phosphorothioate linkages in the 5′ and/or 3′ terminal LNA dinucleotides may be independently or dependently selected from Sp or Rp phosphosphorothiate linkages.

In some embodiments where the oligomer comprises both a 5′ and a 3′ terminal LNA dinucleotide, the oligomer may be a gapmer oligonucleotide, and as such comprise a central region of at least 5 or more contiguous DNA nucleosides, such as 6, 7, 8, 9, 10, 11, 12, 13, 14 or 15 contiguous DNA nucleotides. Gapmers comprising stereospecific DNA phosphorothioate units are known in the art (e.g. see Wan et al, NAR November 2014). In some embodiments the DNA gap region comprises at least one stereospecific PS linkage between contiguous DNA units. In some embodiments all of the phosphorothioate linkages in the DNA gap region are stereospecific phosphorothioate linkages. When considering whether a phosphorothioate linkage is part of the gap region, it is considered that due to standard oligonucleotide synthesis methods which proceed in a 5′-3′ direction that the internucleoside linkage between the 5′ wing and the first DNA nucleoside of the gap is part of the wing as the internucleoside linkage originates from the wing monomer, whereas the internucleoside linkage between the 3′ DNA nucleoside of the gap and the 5′ nucleoside of the 3′ wing region is part of the gap region.

In some embodiments the oligomer of the invention is e.g. a gapmer where all the internucleoside linkages between LNA units or between LNA and 2′substituted nucleoside units are stereospecific phosphorothioate linkages. In some embodiments the oligomer of the invention, which may be a gapmer, has all the internucleoside linkages between LNA units or between LNA and 2′substituted nucleoside units being either stereospecific phosphorothioate linkages or non-phosphorothioate linkages, such as phosphodiester linkages.

### Screening Methods

The invention provides for a method of reducing the toxicity of a stereo unspecified phosphorothioate oligonucleotide sequence, comprising the steps of:

a. Providing a stereo unspecified phosphorothioate oligonucleotide (the parent) which has a toxicity phenotype in vivo or in vitro

b. Creating a library of stereo specified phosphorothioate oligonucleotides (the children), retaining the core nucleobase sequence of the parent gapmer oligonucleotide

c. Screening the library created in step b. in an in vivo or in vitro toxicity assay to

d. Identify one or more stereo specified phosphorothioate oligonucleotides which have a reduced toxicity as compared to the stereo unspecified phosphorothioate oligonucleotide.

The stereo specified phosphorothioate oligonucleotides may be as according to the oligonucleotides of the invention, as disclosed herein. In some embodiments, the parent oligonucleotide is a gapmer oligonucleotide, such as a LNA gapmer oligonucleotide as disclosed herein. In some embodiments, the library of stereo specified phosphorothioate oligonucleotides comprises of at least 2, such as at least 5 or at least 10 or at least 15 or at least 20 stereodefined phosphorothioate oligonucleotides.

The screening method may further comprise a step of screening the children oligonucleotides for at least one other functional parameter, for example one or more of RNaseH recruitment activity, RNase H cleavage specificity, biodistribution, target specificity, target binding affinity, and/or in vivo or in vitro potency.

The method of the invention may therefore be used to reduce the toxicity associated with the parent oligonucleotide. Toxicity of oligonucleotides may be evaluated in vitro or in vivo. In vitro assays include the caspase assay (see e.g. the caspase assays disclosed in WO2005/023995) or hepatocyte toxicity assays (see e.g. Soldatow et al., Toxicol Res (Camb). 2013 Jan. 1; 2(1): 23-39.). In vivo toxicities are often identified in the pre-clinical screening, for example in mouse or rat. In vivo toxicity be for hepatotoxicity, which is typically measured by analysing liver transaminase levels in blood serum, e.g. ALT and/or AST, or may for example be nephrotoxicity, which may be assayed by measuring a molecular marker for kidney toxicity, for example blood serum creatinine levels, or levels of the kidney injury marker mRNA, kim-1. Cellular ATP levels may be used to determine cellular toxicity, such as hepatotoxicity or nephrotoxicity.

The selected child oligonucleotides identified by the screening method are therefore safer effective antisense oligonucleotides.

### Stereocontrolled Monomer

A stereocontrolled monomer is a monomer used in oligonucleotide synthesis which confers a stereodefined phosphorothioate internucleoside linkage in the oligonucleotide, i.e. either the Sp or Rp. In some embodiments the monomer may be a amidite such as a phosphoramidite. Therefore monomer may, in some embodiments be a stereocontrolling/controlled amidite, such as a stereocontrolling/controlled phosphoramidite. Suitable monomers are provided in the examples, or in the Oka et al., J. AM. CHEM. SOC. 2008, 130, 16031-16037 9 16031. See also WO10064146, WO 11005761, WO 13012758, WO 14010250, WO 14010718, WO 14012081, and WO 15107425. The term stereocontrolled/stereocontrolling are used interchangeably herein and may also be referred to stereospecific/stereospecified or stereodefined monomers.

As the stereocontrolled monomer may therefore be referred to as a stereocontrolled “phosphorothioate” monomer. The term stereocontrolled and stereocontrolling are used interchangeably herein. In some embodiments, a stereocontrolling monomer, when used with a sulfarizing agent during oligonucleotide synthesis, produces a stereodefined internucleoside linkage on the 3′ side of the newly incorporated nucleoside (or 5′-side of the grown oligonucleotide chain).

Gap Regions with Stereodefined Phosphorothioate Linkages

As reported in Wan et al., there is little benefit is introducing fully Rp or fully Sp gap regions in a gapmer, as compared to a random racemic mixture of phosphorothioate linkages. The present invention is based upon the surprising benefit that the introduction of at least one stereodefined phosphorothioate linkage may substantially improve the biological properties of an oligonucleotide, e.g. see under advantages. This may be achieved by either introducing one or a number, e.g. 2, 3, 4, 5, 6, 7, 8, 9, 10, 11, 12, 13 or 14 stereodefined phosphorothioate linkages, or by stereo specifying all the phosphorothioate linkages in the gap region.

In some embodiments, only 1, 2, 3, 4 or 5 of the internucleoside linkages of the central region (Y′) are stereoselective phosphorothioate linkages, and the remaining internucleoside linkages are randomly Rp or Sp.

In some embodiments, all of the internucleoside linkages of the central region (Y′) are stereoselective phosphorothioate linkages.

In some embodiments, the central region (Y′) comprises at least 5 contiguous phosphorothioate linked DNA nucleoside. In some embodiments, the central region is at least 8 or 9 DNA nucleosides in length. In some embodiments, the central region is at least 10 or 11 DNA nucleosides in length. In some embodiments, the central region is at least 12 or 13 DNA nucleosides in length. In some embodiments, the central region is at least 14 or 15 DNA nucleosides in length.

### Stereo-Selective DNA Motifs

We have previously identified that certain DNA dinucleotides may contribute to the toxicity profile of antisense oligonucleotides (Hagedorn et al., Nucleic Acid Therapeutics 2013, 23; 302-310). In some embodiments of the invention, the toxicity of the DNA dinucleotides in antisense oligonucleotides, such as the LNA gapmer oligonucleotides described herein, may be modulated via introducing stereoselective phosphorothioate internucleoside linkages between the DNA nucleosides of DNA dinucleotides, particularly dinucleotides which are known to contribute to toxicity, e.g. hepatotoxicity. In some embodiments the oligonucleotide of the invention comprises a DNA dinucleotide motif selected from the group consisting of cc, tg, tc, ac, tt, gt, ca and gc, wherein the internucleoside linkage between the DNA nucleosides of the dinucleotide is a stereodefined phosphorothioate linkage such as either a Sp or a Rp phosphorothioate internucleoside linkage. Typically such dinucleotides may be within the gap region of a gapmer oligonucleotide, such as a LNA gapmer oligonucleotide. In some embodiments the oligonucleotide of the invention comprises at least 2, such as at least 3 dinucleotides dependently or independently selected from the above list of DNA dinucleotide motifs.

### RNAse Recruitment

It is recognised that an oligomeric compound may function via non RNase mediated degradation of target mRNA, such as by steric hindrance of translation, or other methods, In some embodiments, the oligomers of the invention are capable of recruiting an endoribonuclease (RNase), such as RNase H.

It is preferable such oligomers, comprise a contiguous nucleotide sequence (region Y′), comprises of a region of at least 6, such as at least 7 consecutive nucleotide units, such as at least 8 or at least 9 consecutive nucleotide units (residues), including 7, 8, 9, 10, 11, 12, 13, 14, 15 or 16 consecutive nucleotides, which, when formed in a duplex with the complementary target RNA is capable of recruiting RNase. The contiguous sequence which is capable of recruiting RNAse may be region Y′ as referred to in the context of a gapmer as described herein. In some embodiments the size of the contiguous sequence which is capable of recruiting RNAse, such as region Y′, may be higher, such as 10, 11, 12, 13, 14, 15, 16, 17, 18, 19 or 20 nucleotide units.

EP 1 222 309 provides in vitro methods for determining RNaseH activity, which may be used to determine the ability to recruit RNaseH. A oligomer is deemed capable of recruiting RNase H if, when provided with the complementary RNA target, it has an initial rate, as measured in pmol/l/min, of at least 1%, such as at least 5%, such as at least 10% or, more than 20% of the of the initial rate determined using DNA only oligonucleotide, having the same base sequence but containing only DNA monomers, with no 2′ substitutions, with phosphorothioate linkage groups between all monomers in the oligonucleotide, using the methodology provided by Example 91-95 of EP 1 222 309.

In some embodiments, an oligomer is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

In other embodiments, an oligomer is deemed capable of recruiting RNaseH if, when provided with the complementary RNA target, and RNaseH, the RNaseH initial rate, as measured in pmol/l/min, is at least 20%, such as at least 40%, such as at least 60%, such as at least 80% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

Typically the region of the oligomer which forms the consecutive nucleotide units which, when formed in a duplex with the complementary target RNA is capable of recruiting RNase consists of nucleotide units which form a DNA/RNA like duplex with the RNA target. The oligomer of the invention, such as the first region, may comprise a nucleotide sequence which comprises both nucleotides and nucleotide analogues, and may be e.g. in the form of a gapmer, a headmer or a tailmer.

A “headmer” is defined as an oligomer that comprises a region X′ and a region Y′ that is contiguous thereto, with the 5′-most monomer of region Y′ linked to the 3′-most monomer of region X′. Region X′ comprises a contiguous stretch of non-RNase recruiting nucleoside analogues and region Y′ comprises a contiguous stretch (such as at least 7 contiguous monomers) of DNA monomers or nucleoside analogue monomers recognizable and cleavable by the RNase.

A “tailmer” is defined as an oligomer that comprises a region X′ and a region Y′ that is contiguous thereto, with the 5′-most monomer of region Y linked to the 3′-most monomer of the region X′. Region X′ comprises a contiguous stretch (such as at least 7 contiguous monomers) of DNA monomers or nucleoside analogue monomers recognizable and cleavable by the RNase, and region X′ comprises a contiguous stretch of non-RNase recruiting nucleoside analogues.

In some embodiments, in addition to enhancing affinity of the oligomer for the target region, some nucleoside analogues also mediate RNase (e.g., RNaseH) binding and cleavage. Since a-L-LNA (BNA) monomers recruit RNaseH activity to a certain extent, in some embodiments, gap regions (e.g., region Y′ as referred to herein) of oligomers containing α-L-LNA monomers consist of fewer monomers recognizable and cleavable by the RNaseH, and more flexibility in the mixmer construction is introduced.

### Gapmer Design

In some embodiments, the oligomer of the invention, comprises or is a LNA gapmer. A gapmer oligomer is an oligomer which comprises a contiguous stretch of nucleotides which is capable of recruiting an RNAse, such as RNAseH, such as a region of at least 5, 6 or 7 DNA nucleotides, referred to herein in as region Y′ (Y′), wherein region Y′ is flanked both 5′ and 3′ by regions of affinity enhancing nucleotide analogues, such as from 1-6 affinity enhancing nucleotide analogues 5′ and 3′ to the contiguous stretch of nucleotides which is capable of recruiting RNAse—these regions are referred to as regions X′ (X′) and Z′ (Z′) respectively. Examples of gapmers are disclosed in WO2004/046160, WO2008/113832, and WO2007/146511. The LNA gapmer oligomers of the invention comprise at least one LNA nucleoside in region X′ or Z′, such as at least one LNA nucleoside in region X′ and at least one LNA nucleotide in region Z′. In some embodiments, at least one LNA nucleotide in region X′ or at least one LNA LNA nucleotide in region Z′ comprise a stereodefined phosphorothioate linkage between the LNA nucleoside and a subsequent (3′) nucleoside. In some embodiments, at least one LNA nucleotide in region X′ and at least one LNA nucleotide in region Z′ comprise a stereodefined phosphorothioate linkage between the LNA nucleoside and a subsequent (3′) nucleoside. In some embodiments, all the internucleoside linkages within region X′, optionally including the internucleoside linkage between region X′ and Y′ are stereodefined phosphorothioate linkages. In some embodiments, all the internucleoside linkages within region Z′, optionally including the internucleoside linkage between region Y′ and Z′ are stereodefined phosphorothioate linkages. In some embodiments, all the internucleoside linkages within region X′ and region Z′ and, optionally including the internucleoside linkage between region X′ and Y′ and/or Y′ and Z′ are stereodefined phosphorothioate linkages.

In some embodiments, the monomers which are capable of recruiting RNAse are selected from the group consisting of DNA monomers, alpha-L-LNA monomers, C4′ alkylayted DNA monomers (see PCT/EP2009/050349 and Vester et al., Bioorg. Med. Chem. Lett. 18 (2008) 2296-2300, hereby incorporated by reference), and UNA (unlinked nucleic acid) nucleotides (see Fluiter et al., Mol. Biosyst., 2009, 10, 1039 hereby incorporated by reference). UNA is unlocked nucleic acid, typically where the C2-C3 C—C bond of the ribose has been removed, forming an unlocked “sugar” residue. Preferably the gapmer comprises a (poly)nucleotide sequence of formula (5′ to 3′), X′—Y′—Z′, wherein; region X′ (X′) (5′ region) consists or comprises of at least one high affinity nucleotide analogue, such as at least one LNA unit, such as from 1-6 affinity enhancing nucleotide analogues, such as LNA units, and; region Y′ (Y′) consists or comprises of at least five consecutive nucleotides which are capable of recruiting RNAse (when formed in a duplex with a complementary RNA molecule, such as the mRNA target), such as DNA nucleotides, and; region Z′ (Z′) (3′ region) consists or comprises of at least one high affinity nucleotide analogue, such as at least one LNA unit, such as from 1-6 affinity enhancing nucleotide analogues, such as LNA units.

In some embodiments, region X′ comprises or consists of 1, 2, 3, 4, 5 or 6 LNA units, such as 2-5 LNA units, such as 3 or 4 LNA units; and/or region Z′ consists or comprises of 1, 2, 3, 4, 5 or 6 LNA units, such as from 2-5 LNA units, such as 3 or 4 LNA units.

In some embodiments, region X′ may comprises of 1, 2, 3, 4, 5 or 6 2′ substituted nucleotide analogues, such as 2′MOE; and/or region Z′ comprises of 1, 2, 3, 4, 5 or 6 2′substituted nucleotide analogues, such as 2′MOE units.

In some embodiments, the substituent at the 2′ position is selected from the group consisting of F; CF₃, CN, N₃, NO, NO₂, O-, S-, or N-alkyl; O-, S-, or N-alkenyl; O-, S- or Nalkynyl; or O-alkyl-O-alkyl, O-alkyl-N-alkyl or N-alkyl-O-alkyl wherein the alkyl, alkenyl and alkynyl may be substituted or unsubstituted C₁-C₁₀ alkyl or C₂-C₁₀ alkenyl and alkynyl. Examples of 2′ substituents include, and are not limited to, O(CH₂) OCH₃, and O(CH₂) NH₂, wherein n is from 1 to about 10, e.g. MOE, DMAOE, DMAEOE.

In some embodiments Y′ consists or comprises of 5, 6, 7, 8, 9, 10, 11 or 12 consecutive nucleotides which are capable of recruiting RNAse, or from 6-10, or from 7-9, such as 8 consecutive nucleotides which are capable of recruiting RNAse. In some embodiments region Y′ consists or comprises at least one DNA nucleotide unit, such as 1-12 DNA units, preferably from 4-12 DNA units, more preferably from 6-10 DNA units, such as from 7-10 DNA units, such as 8, 9 or 10 DNA units.

In some embodiments region X′ consist of 3 or 4 nucleotide analogues, such as LNA, region X′ consists of 7, 8, 9 or 10 DNA units, and region Z′ consists of 3 or 4 nucleotide analogues, such as LNA. Such designs include (X′—Y′—Z′) 3-10-3, 3-10-4, 4-10-3, 3-9-3, 3-9-4, 4-9-3, 3-8-3, 3-8-4, 4-8-3, 3-7-3, 3-7-4, 4-7-3.

Further gapmer designs are disclosed in WO2004/046160, which is hereby incorporated by reference. WO2008/113832, which claims priority from U.S. provisional application 60/977,409 hereby incorporated by reference, refers to ‘shortmer’ gapmer oligomers. In some embodiments, oligomers presented here may be such shortmer gapmers.

In some embodiments the oligomer, e.g. region X′, is consisting of a contiguous nucleotide sequence of a total of 10, 11, 12, 13 or 14 nucleotide units, wherein the contiguous nucleotide sequence comprises or is of formula (5′-3′), X′—Y′—Z′ wherein; X′ consists of 1, 2 or 3 affinity enhancing nucleotide analogue units, such as LNA units; Y′ consists of 7, 8 or 9 contiguous nucleotide units which are capable of recruiting RNAse when formed in a duplex with a complementary RNA molecule (such as a mRNA target); and Z′ consists of 1, 2 or 3 affinity enhancing nucleotide analogue units, such as LNA units.

In some embodiments the oligomer, comprises of a contiguous nucleotide sequence of a total of 10, 11, 12, 13, 14, 15 or 16 nucleotide units, wherein the contiguous nucleotide sequence comprises or is of formula (5′-3′), X′—Y′—Z′ wherein; X′ comprises of 1, 2, 3 or 4 LNA units; Y′ consists of 7, 8, 9 or 10 contiguous nucleotide units which are capable of recruiting RNAse when formed in a duplex with a complementary RNA molecule (such as a mRNA target) e.g. DNA nucleotides; and Z′ comprises of 1, 2, 3 or 4 LNA units.

In some embodiments X′ consists of 1 LNA unit. In some embodiments X′ consists of 2 LNA units. In some embodiments X′ consists of 3 LNA units. In some embodiments Z′ consists of 1 LNA units. In some embodiments Z′ consists of 2 LNA units. In some embodiments Z′ consists of 3 LNA units. In some embodiments Y′ consists of 7 nucleotide units. In some embodiments Y′ consists of 8 nucleotide units. In some embodiments Y′ consists of 9 nucleotide units. In certain embodiments, region Y′ consists of 10 nucleoside monomers. In certain embodiments, region Y′ consists or comprises 1-10 DNA monomers. In some embodiments Y′ comprises of from 1-9 DNA units, such as 2, 3, 4, 5, 6, 7, 8 or 9 DNA units. In some embodiments Y′ consists of DNA units. In some embodiments Y′ comprises of at least one LNA unit which is in the alpha-L configuration, such as 2, 3, 4, 5, 6, 7, 8 or 9 LNA units in the alpha-L-configuration. In some embodiments Y′ comprises of at least one alpha-L-oxy LNA unit or wherein all the LNA units in the alpha-L-configuration are alpha-L-oxy LNA units. In some embodiments the number of nucleotides present in X′—Y′—Z′ are selected from the group consisting of (nucleotide analogue units—region Y′—nucleotide analogue units): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, 3-10-1. In some embodiments the number of nucleotides in X′—Y′—Z′ are selected from the group consisting of: 2-7-1, 1-7-2, 2-7-2, 3-7-3, 2-7-3, 3-7-2, 3-7-4, and 4-7-3. In certain embodiments, each of regions X′ and Y′ consists of three LNA monomers, and region Y′ consists of 8 or 9 or 10 nucleoside monomers, preferably DNA monomers. In some embodiments both X′ and Z′ consists of two LNA units each, and Y′ consists of 8 or 9 nucleotide units, preferably DNA units. In various embodiments, other gapmer designs include those where regions X′ and/or Z′ consists of 3, 4, 5 or 6 nucleoside analogues, such as monomers containing a 2′-O-methoxyethyl-ribose sugar (2′-MOE) or monomers containing a 2′-fluoro-deoxyribose sugar, and region Y′ consists of 8, 9, 10, 11 or 12 nucleosides, such as DNA monomers, where regions X′—Y′—Z′ have 3-9-3, 3-10-3, 5-10-5 or 4-12-4 monomers. Further gapmer designs are disclosed in WO 2007/146511A2, hereby incorporated by reference.

In the gapmer designs reported herein the gap region (Y′) may comprise one or more stereospecific phosphorothaiote linkage, and the remaining internucleoside linkages of the gap region may e.g. be non-stereospecific internucleoside linkages, or may also be stereodefined phosphorothioate linkages. It is recognized that whilst the disruption of the gap region (G) with a beta-D-LNA, such as beta-D-oxy LNA or ScET nucleoside so that the gap region does not comprise at least 5 consecutive DNA (or other RNaseH recruiting nucleosides), usually interferes with RNaseH recruitment, in some embodiments, the disruption of the gap can result in retention of RNaseH recruitment. This is typically achieved by retention of at least 3 or 4 consecutive DNA nucleosides, and is typically sequence or even compound specific—see Rukov et al., NAR published online on Jul. 28, 2015 which discloses “gap-breaker” oligonucleotides which recruit RNaseH which in some instances provide a more specific cleavage of the target RNA. Therefore in some embodiments region G may comprise a beta-D-oxy LNA nucleoside. In some embodiments the gap region G comprises an LNA nucleotide (e.g. beta-D-oxy, ScET or alpha-L-LNA) within the gap region so that the LNA nucleoside is flanked 5′ or 3′ by at least 3 (5′) and 3 (3′) or at least 3 (5′) and 4 (3′) or at least 4(5′) and 3(3′) DNA nucleosides, and wherein the oligonucleotide is capable of recruiting RNaseH.

BNA and LNA Gapmers: The terms BNA and LNA are used interchangeably. A BNA gapmer is a gapmer oligomer (region A) which comprises at least one BNA nucleotide. A LNA gapmer is a gapmer oligomer (region A) which comprises at least one LNA nucleotide. In the gapmer designs reported herein the 5′ region (X′) and or the 3′ region (Z′) may comprise one or more stereospecific phosphorothaiote linkage, and the remaining internucleoside linkages may e.g. be non-stereospecific internucleoside linkages, or may also be stereospecific phosphorothioate linkages. In some embodiments the internucleoside linkages of region X′ and Y′ (the 5′ and 3′ wing regions) are all stereospecific phosphorothioate linkages. In some embodiments the all the internucleoside linkages of the oligomer are stereospecific phosphorothioate linkages.

### Splice Switching Oligomers

In some embodiments, the oligonucleotide is a splice switching oligomer—i.e. an oligomer which targets the pre-mRNA causing an alternative splicing of the pre-mRNA.

Targets for the splice switching oligomer may include TNF receptor, for example the SSO may be one or more of the TNFR SSOs disclosed in WO2007/058894, WO08051306 A1 and PCT/EP2007/061211, hereby incorporated by reference.

Splice switching oligomers are typically (essentially) not capable of recruiting RNaseH and as such gapmer, tailmer or headmer designs are generally not desirable. However, mixmer and totalmers designs are suitable designs for SSOs.

Spice switching oligomers have also been used to target dystrophin deficiency in Duchenne muscular dystrophy.

### Mixmers

Most antisense oligonucleotides are compounds which are designed to recruit RNase enzymes (such as RNaseH) to degrade their intended target. Such compounds include DNA phosphorothioate oligonucleotides and gapmer, headmers and tailmers. These compounds typically comprise a region of at least 5 or 6 DNA nucleotides, and in the case of gapmers are flanked on either side by affinity enhancing nucleotide analogues. The oligomers of the present invention may operate via an RNase (such as RNaseH) independent mechanism. Examples of oligomers which operate via a non-RNaseH (or non-RNase) mechanism are mixmers and totalmers.

The term ‘mixmer’ refers to oligomers which comprise both naturally and non-naturally occurring nucleotides, where, as opposed to gapmers, tailmers, and headmers there is no contiguous sequence of more than 5, and in some embodiments no more than 4 consecutive, such as no more than three consecutive, naturally occurring nucleotides, such as DNA units. In some embodiments, the mixmer does not comprise more than 5 consecutive nucleoside analogues, such as BNA (LNA), and in some embodiments no more than 4 consecutive, such as no more than three consecutive, consecutive nucleoside analogues, such as BNA (LNA). In such mixmers the remaining nucleosides may, for example be DNA nucleosides, and/or in non-bicyclic nucleoside analogues, such as those referred to herein, for example, 2′ substituted nucleoside analogues, such as 2′-O-MOE and or 2′fluoro.

In some embodiments, the substituent at the 2′ position is selected from the group consisting of F; CF₃, CN, N₃, NO, NO₂, O-, S-, or N-alkyl; O-, S-, or N-alkenyl; O-, S- or Nalkynyl; or O-alkyl-O-alkyl, O-alkyl-N-alkyl or N-alkyl-O-alkyl wherein the alkyl, alkenyl and alkynyl may be substituted or unsubstituted C₁-C₁₀ alkyl or C₂-C₁₀ alkenyl and alkynyl. Examples of 2′ substituents include, and are not limited to, O(CH₂), OCH₃, and O(CH₂), NH₂, wherein n is from 1 to about 10, MOE, DMAOE, DMAEOE.

The oligomer according to the invention maybe mixmers—indeed various mixmer designs are highly effective as oligomer or first region thereof, particularly when targeting microRNA (antimiRs), microRNA binding sites on mRNAs (Blockmirs) or as splice switching oligomers (SSOs). See for example WO2007/112754 (LNA-AntimiRs™), WO2008/131807 (LNA splice switching oligos),

In some embodiments, the oligomer or mixmer may comprise of BNA and 2′ substituted nucleoside analogues, optionally with DNA nucleosides—see for example see WO07027894 and WO2007/112754 which are hereby incorporated by reference. Specific examples include oligomers or first regions which comprise LNA, 2′-O-MOE and DNA, LNA, 2′fluoro and 2′-O-MOE, 2′-O-MOE and 2′fluoro, 2′-O-MOE and 2′fluoro and LNA, or LNA and 2′-O-MOE and LNA and DNA.

In some embodiments, the oligomer or mixmer comprises or consists of a contiguous nucleotide sequence of repeating pattern of nucleotide analogue and naturally occurring nucleotides, or one type of nucleotide analogue and a second type of nucleotide analogues. The repeating pattern, may, for instance be every second or every third nucleotide is a nucleotide analogue, such as BNA (LNA), and the remaining nucleotides are naturally occurring nucleotides, such as DNA, or are a 2′substituted nucleotide analogue such as 2′MOE of 2′fluoro analogues as referred to herein, or, in some embodiments selected form the groups of nucleotide analogues referred to herein. It is recognised that the repeating pattern of nucleotide analogues, such as LNA units, may be combined with nucleotide analogues at fixed positions—e.g. at the 5′ or 3′ termini.

In some embodiments the first nucleotide of oligomer or mixmer, counting from the 3′ end, is a nucleotide analogue, such as an LNA nucleotide.

In some embodiments, which maybe the same or different, the second nucleotide of the oligomer or mixmer, counting from the 3′ end, is a nucleotide analogue, such as an LNA nucleotide.

In some embodiments, which maybe the same or different, the seventh and/or eighth nucleotide of the oligomer or mixmer In some embodiments, which maybe the same or different, the ninth and/or the tenth nucleotides of the oligomer or mixmer, counting from the 3′ end, are nucleotide analogues, such as LNA nucleotides.

In some embodiments, which maybe the same or different, the 5′ terminal of olifgmer or mixmer is a nucleotide analogue, such as an LNA nucleotide.

The above design features may, in some embodiments be incorporated into the mixmer design, such as antimiR mixmers.

In some embodiments, the oligomer or mixmer does not comprise a region of more than 4 consecutive DNA nucleotide units or 3 consecutive DNA nucleotide units. In some embodiments, the mixmer does not comprise a region of more than 2 consecutive DNA nucleotide units.

In some embodiments, the oligomer or mixmer comprises at least a region consisting of at least two consecutive nucleotide analogue units, such as at least two consecutive LNA units.

In some embodiments, the oligomer or mixmer comprises at least a region consisting of at least three consecutive nucleotide analogue units, such as at least three consecutive LNA units.

In some embodiments, the oligomer or mixmer of the invention does not comprise a region of more than 7 consecutive nucleotide analogue units, such as LNA units. In some embodiments, the oligomer or mixmer of the invention does not comprise a region of more than 6 consecutive nucleotide analogue units, such as LNA units. In some embodiments, the oligomer or mixmer of the invention does not comprise a region of more than 5 consecutive nucleotide analogue units, such as LNA units. In some embodiments, the oligomer or mixmer of the invention does not comprise a region of more than 4 consecutive nucleotide analogue units, such as LNA units. In some embodiments, the oligomer or mixmer of the invention does not comprise a region of more than 3 consecutive nucleotide analogue units, such as LNA units. In some embodiments, the oligomer or mixmer of the invention does not comprise a region of more than 2 consecutive nucleotide analogue units, such as LNA units.

The following embodiments may apply to mixmers or totalmer oligomers (e.g. as region A):

The oligomer (e.g. region A) of the invention may, in some embodiments, comprise of at least two alternating regions of LNA and non-LNA nucleotides (such as DNA or 2′ substituted nucleotide analogues).

The oligomer of the invention may, in some embodiments, comprise a contiguous sequence of formula: 5′ ([LNA nucleotides]₁₋₅ and [non-LNA nucleotides]₁₋₄)₂₋₁₂. 3′.

In some embodiments, the 5′ nucleotide of the contiguous nucleotide sequence (or the oligomer) is an LNA nucleotide.

In some embodiments, the 3′ nucleotide of the contiguous nucleotide sequence is a nucleotide analogue, such as LNA, or the 2, 3, 4, 5 3′ nucleotides are nucleotide analogues, such as LNA nucleotides, or other nucleotide analogues which confer enhanced serum stability to the oligomer.

In some embodiments, the contiguous nucleotide sequence of the oligomer has a formula 5′ ([LNA nucleotides]₁₋₅-[non-LNA nucleotides]₁₋₄)₂₋₁₁-[LNA nucleotides]₁₋₅ 3′.

In some embodiments, the contiguous nucleotide sequence of the oligomer has 2, 3 or 4 contiguous regions of LNA and non-LNA nucleotides—e.g. comprises formula 5′ ([LNA nucleotides]₁₋₅ and [non-LNA nucleotides]₁₋₄)₂₋₃, optionally with a further 3′ LNA region [LNA nucleotides]₁₋₅.

In some embodiments, the contiguous nucleotide sequence of the oligomer comprises 5′ ([LNA nucleotides]₁₋₃ and [non-LNA nucleotides]₁₋₃)₂₋₅, optionally with a further 3′ LNA region [LNA nucleotides]₁₋₃.

In some embodiments, the contiguous nucleotide sequence of the oligomer comprises 5′ ([LNA nucleotides]₁₋₃ and [non-LNA nucleotides]₁₋₃)₃, optionally with a further 3′ LNA region [LNA nucleotides]₁₋₃.

In some embodiments the non-LNA nucleotides are all DNA nucleotides.

In some embodiments, the non-LNA nucleotides are independently or dependently selected from the group consisting of DNA units, RNA units, 2′-O-alkyl-RNA units, 2′-OMe-RNA units, 2′-amino-DNA units, and 2′-fluoro-DNA units.

In some embodiments the non-LNA nucleotides are (optionally independently selected from the group consisting of 2′ substituted nucleoside analogues, such as (optionally independently) selected from the group consisting of 2′-O-alkyl-RNA units, 2′-OMe-RNA units, 2′-amino-DNA units, 2′-AP, 2′-FANA, 2′-(3-hydroxy)propyl, and 2′-fluoro-DNA units, and/or other (optionally) sugar modified nucleoside analogues such as morpholino, peptide nucleic acid (PNA), CeNA, unlinked nucleic acid (UNA), hexitol nucleoic acid (HNA). bicyclo-HNA (see e.g. WO2009/100320), In some embodiments, the nucleoside analogues increase the affinity of the first region for its target nucleic acid (or a complementary DNA or RNA sequence). Various nucleoside analogues are disclosed in Freier & Altmann; Nucl. Acid Res., 1997, 25, 4429-4443 and Uhlmann; Curr. Opinion in Drug Development, 2000, 3(2), 293-213, hereby incorporated by reference.

In some embodiments, the non-LNA nucleotides are DNA nucleotides. In some embodiments, the oligomer or contiguous nucleotide sequence comprises of LNA nucleotides and optionally other nucleotide analogues (such as the nucleotide analogues listed under non-LNA nucleotides) which may be affinity enhancing nucleotide analogues and/or nucleotide analogues which enhance serum stability.

In some embodiments, the oligomer or contiguous nucleotide sequence thereof consists of a contiguous nucleotide sequence of said nucleotide analogues.

In some embodiments, the oligomer or contiguous nucleotide sequence thereof consists of a contiguous nucleotide sequence of LNA nucleotides.

In some embodiments, the oligomer or contiguous nucleotide sequence is 8-12, such as 8 10, or 10-20, such as 12-18 or 14-16 nts in length.

In some embodiments, the oligomer or contiguous nucleotide sequence is capable of forming a duplex with a complementary single stranded RNA nucleic acid molecule with phosphodiester internucleoside linkages, wherein the duplex has a Tₘ of at least about 60° C., such as at least 65° C.

Example of a Tₘ Assay: The oligonucleotide: Oligonucleotide and RNA target (PO) duplexes are diluted to 3 mM in 500 ml RNase-free water and mixed with 500 ml 2× Tₘ-buffer (200 mM NaCl, 0.2 mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

### Totalmers

A totalmer is a single stranded oligomer which only comprises non-naturally occurring nucleosides, such as sugar-modified nucleoside analogues.

The first region according to the invention maybe totalmers—indeed various totalmer designs are highly effective as oligomers or first region thereofs, e.g. particularly when targeting microRNA (antimiRs) or as splice switching oligomers (SSOs). In some embodiments, the totalmer comprises or consists of at least one XYX or YXY sequence motif, such as a repeated sequence XYX or YXY, wherein X is LNA and Y is an alternative (i.e. non LNA) nucleotide analogue, such as a 2′-O-MOE RNA unit and 2′-fluoro DNA unit. The above sequence motif may, in some embodiments, be XXY, XYX, YXY or YYX for example.

In some embodiments, the totalmer may comprise or consist of a contiguous nucleotide sequence of between 7 and 16 nucleotides, such as 9, 10, 11, 12, 13, 14, or 15 nucleotides, such as between 7 and 12 nucleotides.

In some embodiments, the contiguous nucleotide sequence of the totolmer comprises of at least 30%, such as at least 40%, such as at least 50%, such as at least 60%, such as at least 70%, such as at least 80%, such as at least 90%, such as 95%, such as 100% BNA (LNA) units. The remaining units may be selected from the non-LNA nucleotide analogues referred to herein in, such those selected from the group consisting of 2′-O_alkyl-RNA unit, 2′-OMe-RNA unit, 2′-amino-DNA unit, 2′-fluoro-DNA unit, LNA unit, PNA unit, HNA unit, INA unit, and a 2′MOE RNA unit, or the group 2′-OMe RNA unit and 2′-fluoro DNA unit.

In some embodiments the totalmer consist or comprises of a contiguous nucleotide sequence which consists only of LNA units. In some embodiments, the totalmer, such as the LNA totalmer, is between 7-12 nucleoside units in length. In some embodiments, the totalmer (as the oligomer or first region thereof) may be targeted against a microRNA (i.e. be antimiRs)—as referred to WO2009/043353, which are hereby incorporated by reference.

In some embodiments, the oligomer or contiguous nucleotide sequence comprises of LNA nucleotides and optionally other nucleotide analogues which may be affinity enhancing nucleotide analogues and/or nucleotide analogues which enhance serum stability.

In some embodiments, the oligomer or contiguous nucleotide sequence thereof consists of a contiguous nucleotide sequence of said nucleotide analogues.

In some embodiments, the oligomer or contiguous nucleotide sequence thereof consists of a contiguous nucleotide sequence of LNA nucleotides.

### MicroRNA Modulation Via the Oligomer of the Invention

In some embodiments, the oligomer an oligomer, such as an LNA-antimiR® (an LNA mixmer or totalmer), which comprises or consists of a contiguous nucleotide sequence which is corresponds to or is fully complementary to a microRNA sequence, such as a mature microRNA or part thereof. The use of the present invention in controlling the in vivo activity of microRNA is considered of primary importance due to the fact that microRNAs typically regulate numerous mRNAs in the subject. The ability to inactivate therapeutic antimiRs is therefore very desirable.

Numerous microRNAs are related to a number of diseases—see WO2009/043353 for example. The oligomer may in some embodiments, target (i.e. comprises or consists of a contiguous nucleotide sequence which is fully complementary to (a corresponding region of) a microRNA. The microRNA may be a liver expressed microRNA, such as microRNA-21, microRNA-221, miR-122 or miR-33 (miR33a & miR-33b).

Hence, some aspects of the invention relates to the treatment of a disease associated with the expression of microRNAs In some embodiments the oligomer or first region thereof according to the invention, consists or comprises of a contiguous nucleotide sequence which corresponds to or is fully complementary to a microRNA sequence, such as a mature microRNA sequence, such as the human microRNAs published in miRBase (http://microrna.sangerac.uk/cd-bin/sequences/mirna_summary.pl?org=hsa). In some embodiment the microRNA is a viral microRNA. At the time of writing, in miRbase 19, there are 1600 precursors and 2042 mature human miRNA sequences in miRBase which are all hereby incorporated by reference, including the mature microRNA sequence of each human microRNA. In some embodiments the oligomer according to the invention, consists or comprises of a contiguous nucleotide sequence which corresponds to or is fully complementary to hsa-miR122 (NR_029667.1 GI:262205241), such as the mature has-miR-122. In some embodiments the oligomer according to the invention, consists or comprises of a contiguous nucleotide sequence which corresponds to or is fully complementary to hsa-miR122 (NR_029667.1 GI:262205241), such as the mature has-miR-122 across the length of the oligomer.

In some embodiments when the oligomer or first region thereof targets miR-122, the oligomer is for the use in the treatment of hepatitis C infection.

In some embodiments when the oligomer targets hsa-miR-33, such as hsa-miR-33a (GUGCAUUGUAGUUGCAUUGCA) or hsa-miR-33b (GUGCAUUGCUGUUGCAUUGC), for example in use in the treatment of a metabolic disease, such as metabolic syndrome, athersosclerosis, hypercholesterolemia and related disorders. See Najafi-Shoushtar et al, Science 328 1566-1569, Rayner et al., Science 328 (1570-1573), Horie et al., J Am Heart Assoc. 2012, Dec. 1(6). Other liver expressed microRNA which are indicated in metabolic diseases, include miR-758, miR-10b, miR-26 and miR-106b, which are known to directly modulate cholesterol efflux (see Dávalos & Fernández-Hernando, Pharmacol Res. 2013 February) The target may therefore be a microRNA selected from the group consisting of miR-122(MIMAT0004590), miR-33(MIMAT0000091, MIMAT0003301), miR-758 (MIMAT0003879), miR-10b (MIPF0000033), miR-26a (MIMAT0000082) and miR-106b (MIMAT0004672). MicroRNA references are miRBase release 19.

### AntimiR Oligomers

Preferred oligomer or first region thereof ‘antimiR’ designs and oligomers are disclosed in WO2007/112754, WO2007/112753, PCT/DK2008/000344 and U.S. provisional applications 60/979,217 and 61/028,062, all of which are hereby incorporated by reference. In some embodiments, the oligomer or first region thereof is an antimiR which is a mixmer or a totalmer. The term AntimiR may therefore be replaces with the term oligomer.

AntimiR oligomers are oligomers which consist or comprise of a contiguous nucleotide sequence which is fully complementary to, or essentially complementary to (i.e. may comprise one or two mismatches), to a microRNA sequence, or a corresponding sub-sequence thereof. In this regards it is considered that the antimiR may be comprise a contiguous nucleotide sequence which is complementary or essentially complementary to the entire mature microRNA, or the antimiR may be comprise a contiguous nucleotide sequence which is complementary or essentially complementary to a sub-sequence of the mature microRNA or pre-microRNA—such a sub-sequence (and therefore the corresponding contiguous nucleotide sequence) is typically at least 8 nucleotides in length, such as between 8 and 25 nucleotides, such as 9, 10, 11, 12, 13, 14, 15, 16, 17, 18, 19, 20, 21, 22, 23, 24 nucleotides in length, such as between 10-17 or 10-16 nucleotides, such as between 12-15 nucleotides.

Numerous designs of AnitmiRs have been suggested, and typically antimiRs for therapeutic use, such as the contiguous nucleotide sequence thereof comprise one or more nucleotide analogues units.

In some embodiments the antimiR may have a gapmer structure as herein described. However, as explained in WO2007/112754 and WO2007/112753, other designs may be preferable, such as mixmers, or totalmers.

WO2007/112754 and WO2007/112753, both hereby incorporated by reference, provide antimiR oligomers and antimiR oligomer designs where the oligomers which are complementary to mature microRNA

In some embodiments, a subsequence of the antimiR corresponds to the miRNA seed region. In some embodiments, the first or second 3′ nucleobase of the oligomer corresponds to the second 5′ nucleotide of the microRNA sequence.

In some antimiR embodiments, nucleobase units 1 to 6 (inclusive) of the oligomer as measured from the 3′ end the region of the oligomer are complementary to the microRNA seed region sequence.

In some antimiR embodiments, nucleobase units 1 to 7 (inclusive) of the oligomer as measured from the 3′ end the region of the oligomer are complementary to the microRNA seed region sequence.

In some antimiR embodiments, nucleobase units 2 to 7 (inclusive) of the oligomer as measured from the 3′ end the region of the oligomer are complementary to the microRNA seed region sequence.

In some embodiments, the antimiR oligomer comprises at least one nucleotide analogue unit, such as at least one LNA unit, in a position which is within the region complementary to the miRNA seed region. The antimiR oligomer may, in some embodiments comprise at between one and 6 or between 1 and 7 nucleotide analogue units, such as between 1 and 6 and 1 and 7 LNA units, in a position which is within the region complementary to the miRNA seed region.

In some embodiments, the antimiR of the invention is 7, 8 or 9 nucleotides long, and comprises a contiguous nucleotide sequence which is complementary to a seed region of a human or viral microRNA, and wherein at least 80%, such as 85%, such as 90%, such as 95%, such as 100% of the nucleotides are LNA.

In some embodiments, the antimiR of the invention is 7, 8 or 9 nucleotides long, and comprises a contiguous nucleotide sequence which is complementary to a seed region of a human or viral microRNA, and wherein at least 80% of the nucleotides are LNA, and wherein at least 80%, such as 85%, such as 90%, such as 95%, such as 100% of the internucleotide bonds are phosphorothioate bonds.

In some embodiments, the antimiR comprises one or two LNA units in positions three to eight, counting from the 3′ end. This is considered advantageous for the stability of the A-helix formed by the oligo:microRNA duplex, a duplex resembling an RNA:RNA duplex in structure.

The table on pages 48 line 15 to page 51, line 9 of WO2007/112754 provides examples of anti microRNA oligomers (i.e. antimiRs which may be the oligomer or first region thereof) and is hereby specifically incorporated by reference.

### MicroRNA Mimics

In some embodiments the oligomer is in the form of a miRNA mimic which can be introduced into a cell to repress the expression of one or more mRNA target(s). miRNA mimics are typically fully complementary to the full length miRNA sequence. miRNA mimics are compounds comprising a contiguous nucleotide sequence which are homologous to a corresponding region of one, or more, of the miRNA sequences provided or referenced to herein. The use of miRNA mimics or antimiRs can be used to (optionally) further repress the mRNA targets, or to silence (down-regulate) the miRNA, thereby inhibiting the function of the endogenous miRNA, causing derepression and increased expression of the mRNA target.

### Aptamers

In some embodiments the oligomer may be a therapeutic aptamer, a spiegelmer. Please note that aptamers may also be ligands, such as receptor ligands, and may therefore be used as a targeting moiety (i.e. further conjugate). Aptamers (e.g. Spiegelmers) in the context of the present invention as nucleic acids of between 20 and 50 nucleotides in length, which have been selected on the basis of their conformational structure rather than the sequence of nucleotides—they elicit their therapeutic effect by binding with a target protein directly in vivo and they do not, therefore, comprise of the reverse complement of their target—indeed their target is not a nucleic acid but a protein. Specific aptamers which may be the oligomer or first region thereof include Macugen (OSI Pharmaceuticals) or ARC1779, (Archemix, Cambridge, Mass.). In some embodiments, the oligomer or first region thereof is not an aptamer. In some embodiments the oligomer or first region thereof is not an aptamer or a spiegelmer.

The oligomer of the invention comprises at least one stereodefined phosphorothioate linkage. Whilst the majority of compounds used for therapeutic use phosphorothioate internucleotide linkages, it is possible to use other internucleoside linkages. However, in some embodiments all the internucleoside linkages of the oligomer of the invention are phosphorothioate internucleoside linkages. In some embodiments the linkages in the gap region are all phosphorothioate and the internucleoside linkages of the wing regions may be either phosphorothioate or phosphodiester linkages.

The nucleoside monomers of the oligomer described herein are coupled together via [internucleoside] linkage groups. Suitably, each monomer is linked to the 3′ adjacent monomer via a linkage group.

The person having ordinary skill in the art would understand that, in the context of the present invention, the 5′ monomer at the end of an oligomer does not comprise a 5′ linkage group, although it may or may not comprise a 5′ terminal group.

The terms “linkage group” or “internucleotide linkage” are intended to mean a group capable of covalently coupling together two nucleotides. Specific and preferred examples include phosphate groups and phosphorothioate groups.

The nucleotides of the oligomer of the invention or contiguous nucleotides sequence thereof are coupled together via linkage groups. Suitably each nucleotide is linked to the 3′ adjacent nucleotide via a linkage group.

Suitable internucleotide linkages include those listed within WO2007/031091, for example the internucleotide linkages listed on the first paragraph of page 34 of WO2007/031091 (hereby incorporated by reference).

It is, in some embodiments, it is desirable to modify the internucleotide linkage from its normal phosphodiester to one that is more resistant to nuclease attack, such as phosphorothioate or boranophosphate—these two, being cleavable by RNase H, also allow that route of antisense inhibition in reducing the expression of the target gene.

Suitable sulphur (S) containing internucleotide linkages as provided herein may be preferred, such as phosphorothioate or phosphodithioate.

For gapmers, the internucleotide linkages in the oligomer may, for example be phosphorothioate or boranophosphate so as to allow RNase H cleavage of targeted RNA. Phosphorothioate is usually preferred, for improved nuclease resistance and other reasons, such as ease of manufacture.

WO09124238 refers to oligomeric compounds having at least one bicyclic nucleoside (LNA) attached to the 3′ or 5′ termini by a neutral internucleoside linkage. The oligomers of the invention may therefore have at least one bicyclic nucleoside attached to the 3′ or 5′ termini by a neutral internucleoside linkage, such as one or more phosphotriester, methylphosphonate, MMI, amide-3, formacetal or thioformacetal. The remaining linkages may be phosphorothioate.

### Nucleosides and Nucleoside Analogues

In some embodiments, the terms “nucleoside analogue” and “nucleotide analogue” are used interchangeably.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogues” herein. Herein, a single nucleotide (unit) may also be referred to as a monomer or nucleic acid unit.

In field of biochemistry, the term “nucleoside” is commonly used to refer to a glycoside comprising a sugar moiety and a base moiety, and may therefore be used when referring to the nucleotide units, which are covalently linked by the internucleotide linkages between the nucleotides of the oligomer. In the field of biotechnology, the term “nucleotide” is often used to refer to a nucleic acid monomer or unit, and as such in the context of an oligonucleotide may refer to the base—such as the “nucleotide sequence”, typically refer to the nucleobase sequence (i.e. the presence of the sugar backbone and internucleoside linkages are implicit). Likewise, particularly in the case of oligonucleotides where one or more of the internucleoside linkage groups are modified, the term “nucleotide” may refer to a “nucleoside” for example the term “nucleotide” may be used, even when specifiying the presence or nature of the linkages between the nucleosides.

As one of ordinary skill in the art would recognise, the 5′ terminal nucleotide of an oligonucleotide does not comprise a 5′ internucleotide linkage group, although may or may not comprise a 5′ terminal group.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1:

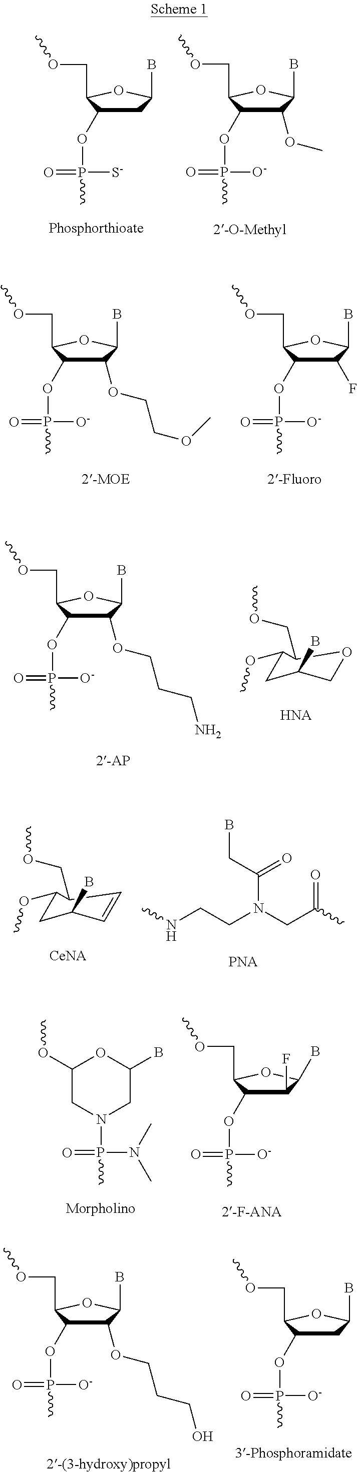

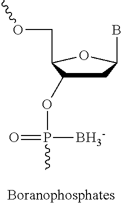

The oligomer may thus comprise or consist of a simple sequence of natural occurring nucleotides—preferably 2′-deoxynucleotides (referred to here generally as “DNA”), but also possibly ribonucleotides (referred to here generally as “RNA”), or a combination of such naturally occurring nucleotides and one or more non-naturally occurring nucleotides, i.e. nucleotide analogues. Such nucleotide analogues may suitably enhance the affinity of the oligomer for the target sequence.

Examples of suitable and preferred nucleotide analogues are provided by WO2007/031091 or are referenced therein.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

In some embodiments, the oligomer comprises at least 1 nucleotide analogue. In some embodiments the oligomer comprises at least 2 nucleotide analogues. In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA.

It will be recognised that when referring to a preferred nucleotide sequence motif or nucleotide sequence, which consists of only nucleotides, the oligomers of the invention which are defined by that sequence may comprise a corresponding nucleotide analogue in place of one or more of the nucleotides present in said sequence, such as LNA units or other nucleotide analogues, which raise the duplex stability/Tₘ of the oligomer/target duplex (i.e. affinity enhancing nucleotide analogues).

Examples of such modification of the nucleotide include modifying the sugar moiety to provide a 2′-substituent group or to produce a bridged (locked nucleic acid) structure which enhances binding affinity and may also provide increased nuclease resistance.

A preferred nucleotide analogue is LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). Most preferred is beta-D-oxy-LNA.

In some embodiments the nucleotide analogues present within the oligomer of the invention (such as in regions X′ and Y′ mentioned herein) are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid—Christensen, 2002. Nucl. Acids. Res. 2002 30: 4918-4925, hereby incorporated by reference) units and 2′MOE units. In some embodiments there is only one of the above types of nucleotide analogues present in the oligomer of the invention, or contiguous nucleotide sequence thereof.

In some embodiments the nucleotide analogues are 2′-O-methoxyethyl-RNA (2′MOE), 2′-fluoro-DNA monomers or LNA nucleotide analogues, and as such the oligonucleotide of the invention may comprise nucleotide analogues which are independently selected from these three types of analogue, or may comprise only one type of analogue selected from the three types. In some embodiments at least one of said nucleotide analogues is 2′-MOE-RNA, such as 2, 3, 4, 5, 6, 7, 8, 9 or 10 2′-MOE-RNA nucleotide units. In some embodiments at least one of said nucleotide analogues is 2′-fluoro DNA, such as 2, 3, 4, 5, 6, 7, 8, 9 or 10 2′-fluoro-DNA nucleotide units.

In some embodiments, the oligomer according to the invention comprises at least one Locked Nucleic Acid (LNA) unit, such as 1, 2, 3, 4, 5, 6, 7, or 8 LNA units, such as from 3-7 or 4 to 8 LNA units, or 3, 4, 5, 6 or 7 LNA units. In some embodiments, all the nucleotide analogues are LNA. In some embodiments, the oligomer may comprise both beta-D-oxy-LNA, and one or more of the following LNA units: thio-LNA, amino-LNA, oxy-LNA, and/or ENA in either the beta-D or alpha-L configurations or combinations thereof. In some embodiments all LNA cytosine units are 5′methyl-Cytosine. In some embodiments of the invention, the oligomer may comprise both LNA and DNA units. Preferably the combined total of LNA and DNA units is 10-25, such as 10-24, preferably 10-20, such as 10-18, even more preferably 12-16. In some embodiments of the invention, the nucleotide sequence of the oligomer, such as the contiguous nucleotide sequence consists of at least one LNA and the remaining nucleotide units are DNA units. In some embodiments the oligomer comprises only LNA nucleotide analogues and naturally occurring nucleotides (such as RNA or DNA, most preferably DNA nucleotides), optionally with modified internucleotide linkages such as phosphorothioate.

The term “nucleobase” refers to the base moiety of a nucleotide and covers both naturally occurring a well as non-naturally occurring variants. Thus, “nucleobase” covers not only the known purine and pyrimidine heterocycles but also heterocyclic analogues and tautomeres thereof.

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

In some embodiments, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

### LNA

The term “LNA” refers to a bicyclic nucleoside analogue, known as “Locked Nucleic Acid”. It may refer to an LNA monomer, or, when used in the context of an “LNA oligonucleotide”, LNA refers to an oligonucleotide containing one or more such bicyclic nucleotide analogues. LNA nucleotides are characterised by the presence of a linker group (such as a bridge) between C2′ and C4′ of the ribose sugar ring—for example as shown as the biradical R⁴*-R²* as described below.

The LNA used in the oligonucleotide compounds of the invention preferably has the structure of the general formula I

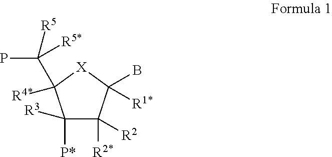

wherein for all chiral centers, asymmetric groups may be found in either R or S orientation;

wherein X is selected from —O—, —S—, —N(R^{N}*)—, —C(R⁶R⁶*)—, such as, in some embodiments —O—;

B is selected from hydrogen, optionally substituted C₁₋₄-alkoxy, optionally substituted C₁₋₄-alkyl, optionally substituted C₁₋₄-acyloxy, nucleobases including naturally occurring and nucleobase analogues, DNA intercalators, photochemically active groups, thermochemically active groups, chelating groups, reporter groups, and ligands; preferably, B is a nucleobase or nucleobase analogue;

P designates an internucleotide linkage to an adjacent monomer, or a 5′-terminal group, such internucleotide linkage or 5′-terminal group optionally including the substituent R⁵ or equally applicable the substituent R⁵*;

P* designates an internucleotide linkage to an adjacent monomer, or a 3′-terminal group;

R⁴* and R²* together designate a bivalent linker group consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z, wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, optionally substituted C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), wherein for all chiral centers, asymmetric groups may be found in either R or S orientation, and;

each of the substituents R¹*, R², R³, R⁵, R⁵*, R⁶ and R^{6′}, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene; wherein R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof. For all chiral centers, asymmetric groups may be found in either R or S orientation.

In some embodiments, R⁴* and R²* together designate a biradical consisting of a groups selected from the group consisting of C(R^{a}R^{b})—C(R^{a}R^{b})—, C(R^{a}R^{b})—O—, C(R^{a}R^{b})—NR^{a}—, C(R^{a}R^{b})—S—, and C(R^{a}R^{b})—C(R^{a}R^{b})—O—, wherein each R^{a} and R^{b} may optionally be independently selected. In some embodiments, R^{a} and R^{b} may be, optionally independently selected from the group consisting of hydrogen and C₁₋₆ alkyl, such as methyl, such as hydrogen.

In some embodiments, R⁴* and R²* together designate the biradical —O—CH(CH₂OCH₃)-(2′O-methoxyethyl bicyclic nucleic acid—Seth at al., 2010, J. Org. Chem)—in either the R- or S-configuration.

In some embodiments, R⁴* and R²* together designate the biradical —O—CH(CH₂CH₃)-(2′O-ethyl bicyclic nucleic acid—Seth at al., 2010, J. Org. Chem).—in either the R- or S-configuration.

In some embodiments, R⁴* and R²* together designate the biradical —O—CH(CH₃)—.—in either the R- or S-configuration. In some embodiments, R⁴* and R²* together designate the biradical —O—CH₂—O—CH₂— —(Seth at al., 2010, J. Org. Chem).

In some embodiments, R⁴* and R²* together designate the biradical —O—NR—CH₃— —(Seth at al., 2010, J. Org. Chem).

In some embodiments, the LNA units have a structure selected from the following group:

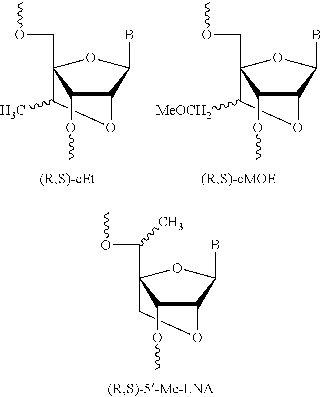

In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. For all chiral centers, asymmetric groups may be found in either R or S orientation.

In some embodiments, R^{1′}, R², R³, R⁵, R⁵* are hydrogen.

In some embodiments, R¹*, R², R³ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. For all chiral centers, asymmetric groups may be found in either R or S orientation.

In some embodiments, R¹*, R², R³ are hydrogen.

In some embodiments, R⁵ and R⁵* are each independently selected from the group consisting of H, —CH₃, —CH₂—CH₃, —CH₂—C—CH₃, and —CH═CH₂. Suitably in some embodiments, either R⁵ or R⁵* are hydrogen, where as the other group (R⁵ or R⁵* respectively) is selected from the group consisting of C₁₋₅ alkyl, C₂₋₆ alkenyl, C₂₋₆ alkynyl, substituted C₁₋₆ alkyl, substituted C₂₋₆ alkenyl, substituted C₂₋₆ alkynyl or substituted acyl (—O(═O)—); wherein each substituted group is mono or poly substituted with substituent groups independently selected from halogen, C₁₋₆ alkyl, substituted C₁₋₆ alkyl, C₂₋₆ alkenyl, substituted C₂₋₆ alkenyl, C₂₋₆alkynyl, substituted C₂₋₆ alkynyl, OJ₁, SJ₁, NJ₁J₂, N₃, COOJ₁, ON, O—C(═O)NJ₁J₂, N(H)C(═NH)NJ₁J₂ or N(H)C(═X)N(H)J₂ wherein X is O or S; and each J₁ and J₂ is, independently, H, C₁₋₆ alkyl, substituted C₁₋₆ alkyl, C₂₋₆ alkenyl, substituted C₂₋₆ alkenyl, C₂₋₆alkynyl, substituted C₂₋₆ alkynyl, C₁₋₆ aminoalkyl, substituted C₁₋₆ aminoalkyl or a protecting group. In some embodiments either R⁵ or R⁵* is substituted C₁₋₆ alkyl. In some embodiments either R⁵ or R⁵* is substituted methylene wherein preferred substituent groups include one or more groups independently selected from F, NJ₁J₂, N₃, ON, OJ₁, SJ₁, O—C(═O)NJ₁J₂, N(H)C(═NH)NJ, J₂ or N(H)C(O)N(H)J₂. In some embodiments each J₁ and J₂ is, independently H or C₁₋₆ alkyl. In some embodiments either R⁵ or R⁵* is methyl, ethyl or methoxymethyl. In some embodiments either R⁵ or R⁵* is methyl. In a further embodiment either R⁵ or R⁵* is ethylenyl. In some embodiments either R⁵ or R⁵* is substituted acyl. In some embodiments either R⁵ or R⁵* is C(═O)NJ₁J₂. For all chiral centers, asymmetric groups may be found in either R or S orientation. Such 5′ modified bicyclic nucleotides are disclosed in WO 2007/134181, which is hereby incorporated by reference in its entirety.

In some embodiments B is a nucleobase, including nucleobase analogues and naturally occurring nucleobases, such as a purine or pyrimidine, or a substituted purine or substituted pyrimidine, such as a nucleobase referred to herein, such as a nucleobase selected from the group consisting of adenine, cytosine, thymine, adenine, uracil, and/or a modified or substituted nucleobase, such as 5-thiazolo-uracil, 2-thio-uracil, 5-propynyl-uracil, 2′thio-thymine, 5-methyl cytosine, 5-thiazolo-cytosine, 5-propynyl-cytosine, and 2,6-diaminopurine.

In some embodiments, R⁴* and R²* together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O—, and —C(R^{a}R^{b})—S—, —C(R^{a}R^{b})—C(R^{c}R^{d})—S—, wherein R^{d}, R^{b}, R^{c}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂). For all chiral centers, asymmetric groups may be found in either R or S orientation.

In a further embodiment R⁴* and R²* together designate a biradical (bivalent group) selected from —CH₂—O—, —CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, and —CH(CH₂—O—CH₃)—O—, and/or, —CH₂—CH₂—, and —CH═CH— For all chiral centers, asymmetric groups may be found in either R or S orientation.

In some embodiments, R⁴* and R²* together designate the biradical C(R^{a}R^{b})—N(R^{c})—O—, wherein R^{a} and R^{b} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen, and; wherein R^{c} is selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* together designate the biradical C(R^{a}R^{b})—O—C(R^{c}R^{d}) —O—, wherein R^{a}, R^{b}, R^{c}, and R^{d} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* form the biradical —CH(Z)—O—, wherein Z is selected from the group consisting of C₁₋₆ alkyl, C₂₋₆ alkenyl, C₂₋₆ alkynyl, substituted C₁₋₆ alkyl, substituted C₂₋₆ alkenyl, substituted C₂₋₆ alkynyl, acyl, substituted acyl, substituted amide, thiol or substituted thio; and wherein each of the substituted groups, is, independently, mono or poly substituted with optionally protected substituent groups independently selected from halogen, oxo, hydroxyl, OJ₁, NJ₁J₂, SJ₁, N₃, OC(═X)J₁, OC(═X)NJ₁J₂, NJ³C(═X)NJ₁J₂ and CN, wherein each J₁, J₂ and J₃ is, independently, H or C₁₋₆ alkyl, and X is O, S or NJ₁. In some embodiments Z is C₁₋₆ alkyl or substituted C₁₋₆ alkyl. In some embodiments Z is methyl. In some embodiments Z is substituted C₁₋₆ alkyl. In some embodiments said substituent group is C₁₋₆alkoxy. In some embodiments Z is CH₃OCH₂—. For all chiral centers, asymmetric groups may be found in either R or S orientation. Such bicyclic nucleotides are disclosed in U.S. Pat. No. 7,399,845 which is hereby incorporated by reference in its entirety. In some embodiments, R¹*, R², R³, R⁵, R⁵* are hydrogen. In some some embodiments, R¹*, R², R³* are hydrogen, and one or both of R⁵, R⁵* may be other than hydrogen as referred to above and in WO 2007/134181.

In some embodiments, R⁴* and R²* together designate a biradical which comprise a substituted amino group in the bridge such as consist or comprise of the biradical —CH₂—N(R^{c})—, wherein R^{c} is C₁₋₁₂ alkyloxy. In some embodiments R⁴* and R²* together designate a biradical —Cq₃q₄-NOR—, wherein q₃ and q₄ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl; wherein each substituted group is, independently, mono or poly substituted with substituent groups independently selected from halogen, OJ₁, SJ₁, NJ₁J₂, COOJ₁, CN, O—C(═O)NJ₁J₂, N(H)C(═NH)N J₁J₂ or N(H)C(═X═N(H)J₂ wherein X is O or S; and each of J₁ and J₂ is, independently, H, C₁₋₆ alkyl, C₂₋₆ alkenyl, C₂₋₆ alkynyl, C₁₋₆ aminoalkyl or a protecting group. For all chiral centers, asymmetric groups may be found in either R or S orientation. Such bicyclic nucleotides are disclosed in WO2008/150729 which is hereby incorporated by reference in its entirety. In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R^{1′}, R², R³, R⁵, R⁵* are hydrogen. In some embodiments, R¹*, R², R³ are hydrogen and one or both of R⁵, R⁵* may be other than hydrogen as referred to above and in WO 2007/134181. In some embodiments R⁴* and R²* together designate a biradical (bivalent group) C(R^{a}R^{b})—C—, wherein R^{a} and R^{b} are each independently halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ₁SJ₁, SOJ₁, SO₂J₁, NJ₁J₂, N₃, ON, C(═O)OJ₁, C(═O)NJ₁J₂, C(═O)J₁, O—C(═O)NJ₁J₂, N(H)O(═NH)NJ₁J₂, N(H)O(═O)NJ₁J₂ or N(H)O(═S)NJ₁J₂; or R^{a} and R^{b} together are ═C(q3)(q4); q₃ and q₄ are each, independently, H, halogen, C₁-C₁₂alkyl or substituted C₁-C₁₂ alkyl; each substituted group is, independently, mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ₁J₂, N₃, ON, C(═O)OJ₁, C(═O)NJ₁J₂, C(═O)J₁, O—C(═O)NJ₁J₂, N(H)O(═O)NJ₁J₂ or N(H)O(═S)NJ₁J₂ and; each J₁ and J₂ is, independently, H, C1-C₆ alkyl, substituted C1-C₆ alkyl, C₂-C₆ alkenyl, substituted C₂-C₆ alkenyl, C₂-C₆ alkynyl, substituted C₂-C₆ alkynyl, C1-C₆ aminoalkyl, substituted C1-C₆ aminoalkyl or a protecting group. Such compounds are disclosed in WO2009006478A, hereby incorporated in its entirety by reference.

In some embodiments, R⁴* and R²* form the biradical -Q-, wherein Q is C(q₁)(q₂)C(q₃)(q₄), C(q₁)═C(q₃), C[═C(q₁)(q₂)]—C(q₃)(q₄) or C(q₁)(q₂)—C[═C(q₃)(q₄)]; q₁, q₂, q₃, q₄ are each independently. H, halogen, C₁₋₁₂ alkyl, substituted C₁₋₁₂ alkyl, C₂₋₁₂ alkenyl, substituted C₁₋₁₂alkoxy, OJ₁, SJ₁, SOJ₁, SO₂J₁, NJ₁J₂, N₃, ON, C(═O)OJ₁, C(═O)—NJ₁J₂, C(═O) J₁, —C(═O)NJ₁J₂, N(H)C(═NH)NJ₁J₂, N(H)C(═O)NJ₁J₂ or N(H)C(═S)NJ₁J₂; each J₁ and J₂ is, independently, H, C₁₋₆ alkyl, C₂₋₆ alkenyl, C₂₋₆ alkynyl, C₁₋₆ aminoalkyl or a protecting group; and, optionally wherein when Q is C(q₁)(q₂)(q₃)(q₄) and one of q₃ or q₄ is CH₃ then at least one of the other of q₃ or q₄ or one of q₁ and q₂ is other than H. In some embodiments, R¹*, R², R³, R⁵, R⁵* are hydrogen. For all chiral centers, asymmetric groups may be found in either R or S orientation. Such bicyclic nucleotides are disclosed in WO2008/154401 which is hereby incorporated by reference in its entirety. In some embodiments, R^{1′}, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆alkoxyl, substituted C₁₋₆alkoxyl, acyl, substituted acyl, C₁₋₆ aminoalkyl or substituted C₁₋₆ aminoalkyl. In some embodiments, R¹*, R², R³, R⁵, R⁵* are hydrogen. In some embodiments, R¹*, R², R³ are hydrogen and one or both of R⁵, R⁵* may be other than hydrogen as referred to above and in WO 2007/134181 or WO2009/067647 (alpha-L-bicyclic nucleic acids analogs).

Further bicyclic nucleoside analogues and their use in antisense oligonucleotides are disclosed in WO2011 115818, WO2011/085102, WO2011/017521, WO09100320, WO10036698, WO09124295 & WO09006478. Such nucleoside analogues may in some aspects be useful in the compounds of present invention.

In some embodiments the LNA used in the oligonucleotide compounds of the invention preferably has the structure of the general formula II:

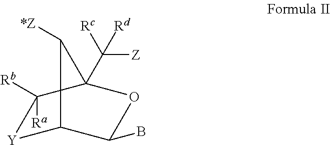

wherein Y is selected from the group consisting of —O—, —CH₂O—, —S—, —NH—, N(Re) and/or —CH₂—; Z and Z* are independently selected among an internucleotide linkage, R^{H}, a terminal group or a protecting group; B constitutes a natural or non-natural nucleotide base moiety (nucleobase), and R^{H} is selected from hydrogen and C₁₋₄-alkyl; R^{a}, R^{b} R^{c}, R^{d} and Re are, optionally independently, selected from the group consisting of hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂); and R^{H} is selected from hydrogen and C₁₋₄-alkyl. In some embodiments R^{a}, R^{b} R^{c}, R^{d} and Re are, optionally independently, selected from the group consisting of hydrogen and C₁₋₆ alkyl, such as methyl. For all chiral centers, asymmetric groups may be found in either R or S orientation, for example, two exemplary stereochemical isomers include the beta-D and alpha-L isoforms, which may be illustrated as follows:

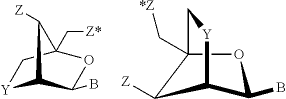

Specific exemplary LNA units are shown below:

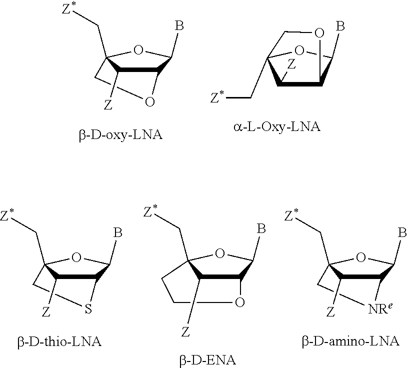

The term “thio-LNA” comprises a locked nucleotide in which Y in the general formula above is selected from S or —CH₂—S—. Thio-LNA can be in both beta-D and alpha-L-configuration.

The term “amino-LNA” comprises a locked nucleotide in which Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which Y in the general formula above represents —O—. Oxy-LNA can be in both beta-D and alpha-L-configuration. The term “ENA” comprises a locked nucleotide in which Y in the general formula above is —CH₂—O— (where the oxygen atom of —CH₂—O— is attached to the 2′-position relative to the base B). Re is hydrogen or methyl.

In some exemplary embodiments LNA is selected from beta-D-oxy-LNA, alpha-L-oxy-LNA, beta-D-amino-LNA and beta-D-thio-LNA, in particular beta-D-oxy-LNA.

### Conjugates

In the context the term “conjugate” is intended to indicate a heterogenous molecule formed by the covalent attachment (“conjugation”) of the oligomer as described herein to one or more non-nucleotide, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol.

Therefore, in various embodiments, the oligomer of the invention may comprise both a polynucleotide region which typically consists of a contiguous sequence of nucleotides, and a further non-nucleotide region. When referring to the oligomer of the invention consisting of a contiguous nucleotide sequence, the compound may comprise non-nucleotide components, such as a conjugate component.

In various embodiments of the invention the oligomeric compound is linked to ligands/conjugates, which may be used, e.g. to increase the cellular uptake of oligomeric compounds. WO2007/031091 provides suitable ligands and conjugates, which are hereby incorporated by reference.

The invention also provides for a conjugate comprising the compound according to the invention as herein described, and at least one non-nucleotide or non-polynucleotide moiety covalently attached to said compound. Therefore, in various embodiments where the compound of the invention consists of a specified nucleic acid or nucleotide sequence, as herein disclosed, the compound may also comprise at least one non-nucleotide or non-polynucleotide moiety (e.g. not comprising one or more nucleotides or nucleotide analogues) covalently attached to said compound.

In some embodiments, the non-nucleotide moiety (conjugate moiety) is selected from the group consisting of carbohydrates, cell surface receptor ligands, drug substances, hormones, a protein, such as an enzyme, an antibody or an antibody fragment or a peptide; a lipophilic substances, polymers, proteins, peptides, toxins (e.g. bacterial toxins), vitamins, viral proteins (e.g. capsids) or combinations thereof moiety such as a lipid, a phospholipid, a sterol; a polymer, such as polyethyleneglycol or polypropylene glycol; a receptor ligand; a small molecule; a reporter molecule; and a non-nucleosidic carbohydrate.

Conjugation (to a conjugate moiety) may enhance the activity, cellular distribution or cellular uptake of the oligomer of the invention. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

The oligomers of the invention may also be conjugated to active drug substances, for example, aspirin, ibuprofen, a sulfa drug, an antidiabetic, an antibacterial or an antibiotic.

In certain embodiments the conjugated moiety is a sterol, such as cholesterol.

In various embodiments, the conjugated moiety comprises or consists of a positively charged polymer, such as a positively charged peptides of, for example from 1-50, such as 2-20 such as 3-10 amino acid residues in length, and/or polyalkylene oxide such as polyethylglycol(PEG) or polypropylene glycol—see WO 2008/034123, hereby incorporated by reference. Suitably the positively charged polymer, such as a polyalkylene oxide may be attached to the oligomer of the invention via a linker such as the releasable inker described in WO 2008/034123.

By way of example, the following GalNAc conjugate moieties may be used in the conjugates of the invention:

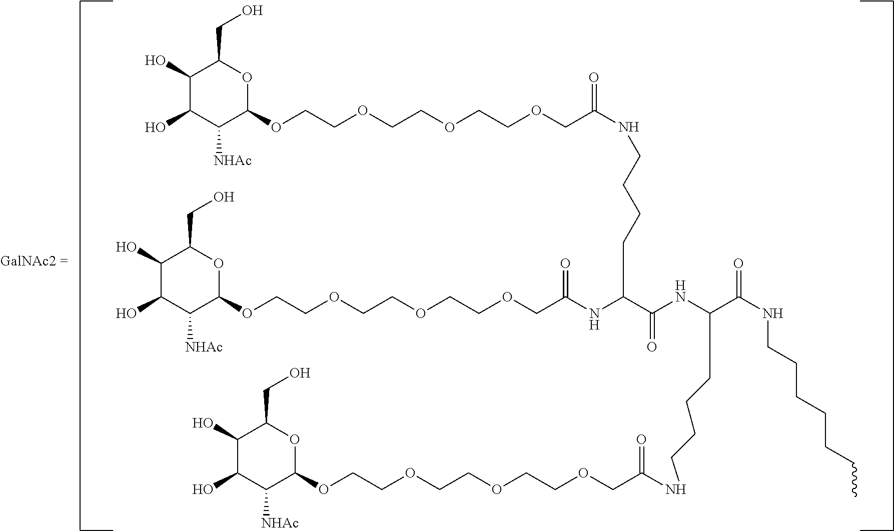

The invention further provides a conjugate comprising the oligomer according to the invention, which comprises at least one non-nucleotide or non-polynucleotide moiety (“conjugated moiety”) covalently attached to the oligomer of the invention. In some embodiments the conjugate of the invention is covalently attached to the oligomer via a biocleavable linker, which, for example may be a region of phosphodiester linked nucleotides, such as 1-5 PO linked DNA nucleosides (WO2014/076195, hereby incorporated by reference). Preferred conjugate groups include carbohydrate conjugates, such as GalNAc conjugates, such as trivalent GalNAc conjugates (e.g. see WO2014/118267, hereby incorporated by reference) or lipophilic conjugates, such as a sterol, e.g. cholesterol (WO2014/076195, hereby incorporated by reference)

### Activated Oligomers

The term “activated oligomer,” as used herein, refers to an oligomer of the invention that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that is preferably hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl. In some embodiments, the functional moiety is self-cleaving. In other embodiments, the functional moiety is biodegradable. See e.g., U.S. Pat. No. 7,087,229, which is incorporated by reference herein in its entirety.

In some embodiments, oligomers of the invention are functionalized at the 5′ end in order to allow covalent attachment of the conjugated moiety to the 5′ end of the oligomer. In other embodiments, oligomers of the invention can be functionalized at the 3′ end. In still other embodiments, oligomers of the invention can be functionalized along the backbone or on the heterocyclic base moiety. In yet other embodiments, oligomers of the invention can be functionalized at more than one position independently selected from the 5′ end, the 3′ end, the backbone and the base.

In some embodiments, activated oligomers of the invention are synthesized by incorporating during the synthesis one or more monomers that is covalently attached to a functional moiety. In other embodiments, activated oligomers of the invention are synthesized with monomers that have not been functionalized, and the oligomer is functionalized upon completion of synthesis. In some embodiments, the oligomers are functionalized with a hindered ester containing an aminoalkyl linker, wherein the alkyl portion has the formula (CH₂)_{w}, wherein w is an integer ranging from 1 to 10, preferably about 6, wherein the alkyl portion of the alkylamino group can be straight chain or branched chain, and wherein the functional group is attached to the oligomer via an ester group (—O—C(O)—(CH₂)₂NH).

In other embodiments, the oligomers are functionalized with a hindered ester containing a (CH₂)₂-sulfhydryl (SH) linker, wherein w is an integer ranging from 1 to 10, preferably about 6, wherein the alkyl portion of the alkylamino group can be straight chain or branched chain, and wherein the functional group attached to the oligomer via an ester group (—O—C(O)—(CH₂)₂SH)

In some embodiments, sulfhydryl-activated oligonucleotides are conjugated with polymer moieties such as polyethylene glycol or peptides (via formation of a disulfide bond).

Activated oligomers containing hindered esters as described above can be synthesized by any method known in the art, and in particular by methods disclosed in PCT Publication No. WO 2008/034122 and the examples therein, which is incorporated herein by reference in its entirety.

In still other embodiments, the oligomers of the invention are functionalized by introducing sulfhydryl, amino or hydroxyl groups into the oligomer by means of a functionalizing reagent substantially as described in U.S. Pat. Nos. 4,962,029 and 4,914,210, i.e., a substantially linear reagent having a phosphoramidite at one end linked through a hydrophilic spacer chain to the opposing end which comprises a protected or unprotected sulfhydryl, amino or hydroxyl group. Such reagents primarily react with hydroxyl groups of the oligomer. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers of the invention have a functionalizing reagent coupled to a hydroxyl group on the backbone of the oligomer. In yet further embodiments, the oligomer of the invention is functionalized with more than one of the functionalizing reagents as described in U.S. Pat. Nos. 4,962,029 and 4,914,210, incorporated herein by reference in their entirety. Methods of synthesizing such functionalizing reagents and incorporating them into monomers or oligomers are disclosed in U.S. Pat. Nos. 4,962,029 and 4,914,210.

In some embodiments, the 5′-terminus of a solid-phase bound oligomer is functionalized with a dienyl phosphoramidite derivative, followed by conjugation of the deprotected oligomer with, e.g., an amino acid or peptide via a Diels-Alder cycloaddition reaction.

In various embodiments, the incorporation of monomers containing 2′-sugar modifications, such as a 2′-carbamate substituted sugar or a 2′-(O-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al., Tetrahedron Letters, 1991, 34, 7171.

In still further embodiments, the oligomers of the invention may have amine-containing functional moieties on the nucleobase, including on the N6 purine amino groups, on the exocyclic N2 of guanine, or on the N4 or 5 positions of cytosine. In various embodiments, such functionalization may be achieved by using a commercial reagent that is already functionalized in the oligomer synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.). In some embodiments in some embodiments

### Compositions

The oligomer of the invention may be used in pharmaceutical formulations and compositions. Suitably, such compositions comprise a pharmaceutically acceptable diluent, carrier, salt or adjuvant. PCT/DK2006/000512 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in PCT/DK2006/000512—which are also hereby incorporated by reference.

### Applications

The oligomers of the invention may be utilized as research reagents for, for example, diagnostics, therapeutics and prophylaxis.

In research, such oligomers may be used to specifically inhibit the synthesis of a target protein (typically by degrading or inhibiting the mRNA and thereby prevent protein formation) in cells and experimental animals thereby facilitating functional analysis of the target or an appraisal of its usefulness as a target for therapeutic intervention.

In diagnostics the oligomers may be used to detect and quantitate a target expression in cell and tissues by northern blotting, in-situ hybridisation or similar techniques.

For therapeutics, an animal or a human, suspected of having a disease or disorder, which can be treated by modulating the expression of a target is treated by administering oligomeric compounds in accordance with this invention. Further provided are methods of treating a mammal, such as treating a human, suspected of having or being prone to a disease or condition, associated with expression of a target by administering a therapeutically or prophylactically effective amount of one or more of the oligomers or compositions of the invention. The oligomer, a conjugate or a pharmaceutical composition according to the invention is typically administered in an effective amount.

The invention also provides for the use of the compound or conjugate of the invention as described for the manufacture of a medicament for the treatment of a disorder as referred to herein, or for a method of the treatment of as a disorder as referred to herein.

The invention also provides for a method for treating a disorder as referred to herein said method comprising administering a compound according to the invention as herein described, and/or a conjugate according to the invention, and/or a pharmaceutical composition according to the invention to a patient in need thereof.

### Medical Indications

The oligomers and other compositions according to the invention can be used for the treatment of conditions associated with over expression or expression of mutated version of the target.

The invention further provides use of a compound of the invention in the manufacture of a medicament for the treatment of a disease, disorder or condition as referred to herein.

Generally stated, one aspect of the invention is directed to a method of treating a mammal suffering from or susceptible to conditions associated with abnormal levels of the target, comprising administering to the mammal and therapeutically effective amount of an oligomer targeted to the target that comprises one or more LNA units. The oligomer, a conjugate or a pharmaceutical composition according to the invention is typically administered in an effective amount.

### Embodiments

The invention is based on the provision of locked nucleic acids (LNAs) also referred to in the art as bicyclic nucleic acids (BNAs), comprising at least one stereospecified phosphorothioate moiety. The invention provides LNA oxazaphospholine Sp monomers. The invention provides LNA oxazaphospholine Rp monomers. The invention provides BNA oxazaphospholine Sp monomers. The invention provides BNA oxazaphospholine Rp monomers.

The invention provides for the use of LNA oxazaphospholine Rp monomers in oligonucleotide synthesis. The invention provides for the use of LNA oxazaphospholine Sp monomers in oligonucleotide synthesis.

In some embodiments the invention provides LNA monomers of formula 1A or 1B:

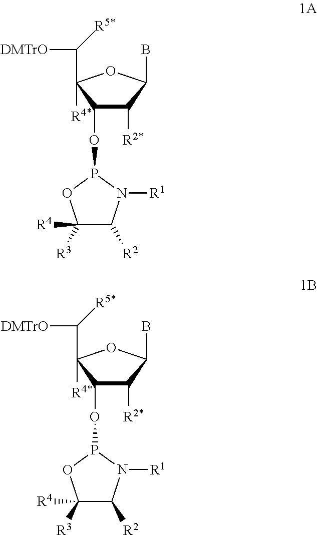

Wherein
B is selected from hydrogen, optionally substituted C₁₋₄-alkoxy, optionally substituted C₁₋₄-alkyl, optionally substituted C₁₋₄-acyloxy, nucleobases including naturally occurring and nucleobase analogues, DNA intercalators, photochemically active groups, thermochemically active groups, chelating groups, reporter groups, and ligands; preferably, B is a nucleobase or nucleobase analogue;
R¹ and R² form a 5 membered heterocyclic ring
R⁴ is hydrogen or C¹-C⁶ alkyl
R³ is phenyl or substituted phenyl,
R⁵* is hydrogen or C¹-C⁶ alkyl,

The biradical R2*-R4* designate a bivalent linker group.

In some embodiments, B may for example be a protected nucleobase, R⁵* may be hydrogen, and R³ may be hydrogen or methyl.

The R⁴*-R²* radical may be as described herein under the description of LNA, such as may be selected from the group consisting of —CH₂—O—, —CH₂—CH₂—O—, CH(CH₃)—O—, —CH₂—S— and CH₂—NR′, wherein R′ is hydrogen of C₁-C₆ alkyl. A preferred radical is —CH₂—O— or —CH₂—CH₂—O—, where optionally R⁵* is hydrogen.

In some embodiments, the LNA monomer is as according to formula 2A or 2B

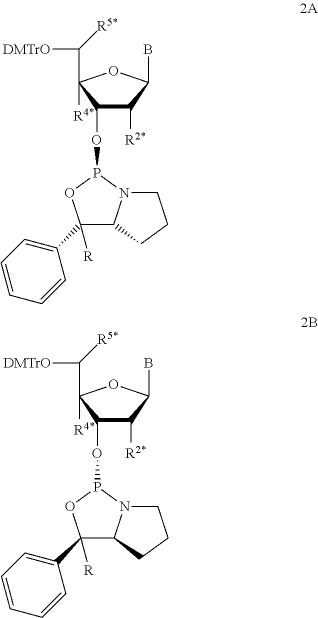

Wherein R⁵*, R4*-R2* and B are is as defined for formula 1A and 1B, and R may for example be hydrogen or C¹-C⁶ alkyl, such as methyl. R⁵* may for example by hydrogen or methyl.

In some embodiments, the LNA monomer of the invention is of formula 3A or 3B:

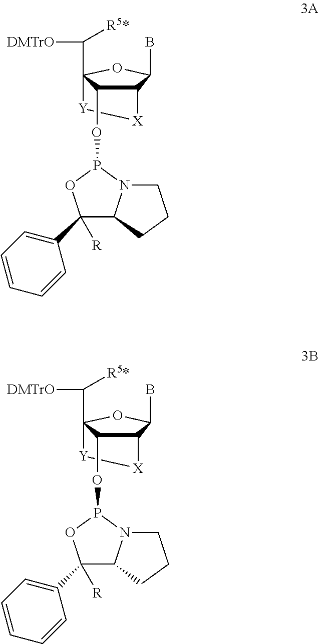

Wherein B and R⁵* may be as described for LNA monomers of formula 1A or 1B above, and wherein Y—X may be as described for the R⁴*-R²* radical herein, such as may be selected from the group consisting of —CH₂—O—, —CH₂—CH₂—O—, CH(CH₃)—O—, —CH₂—S— and CH₂—NR′, wherein R′ is hydrogen of C₁-C₆ alkyl.

In some embodiments, the LNA monomer of the invention is of formula 4A or 4B:

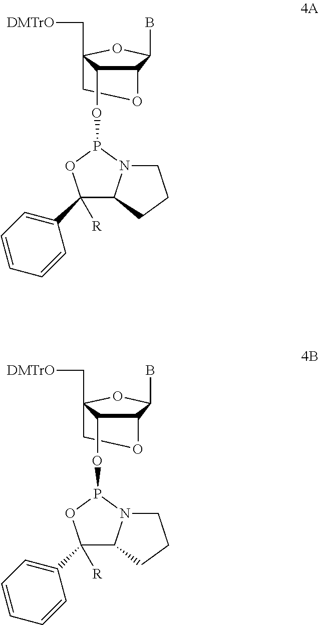

Wherein B may be as described for LNA monomers of formula 1A or 1B above, and R may be hydrogen or C¹-C⁶ alkyl, such as methyl.

In some embodiments the LNA monomers may be as according to formula 5A or 5B

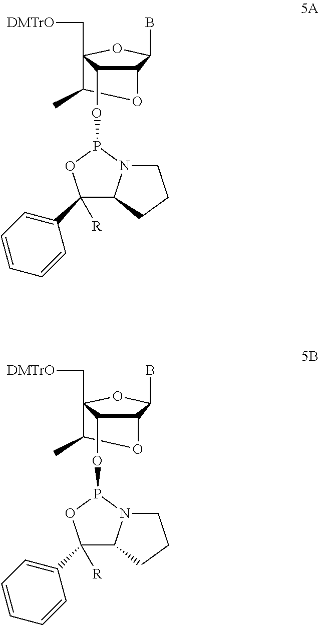

Wherein B may be as described for LNA monomers of formula 1A or 1B above, and R may be hydrogen or C¹-C⁶ alkyl, such as methyl.

In some embodiments the LNA monomers may be as according to formula 6A or 6B

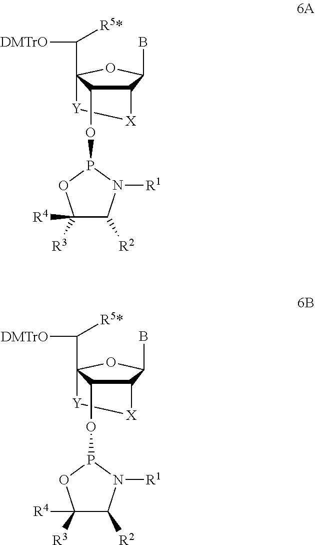

Wherein, B is selected from hydrogen, optionally substituted C₁₋₄-alkoxy, optionally substituted C₁₋₄-alkyl, optionally substituted C₁₋₄-acyloxy, nucleobases including naturally occurring and nucleobase analogues, DNA intercalators, photochemically active groups, thermochemically active groups, chelating groups, reporter groups, and ligands; preferably, B is a nucleobase or nucleobase analogue;

R¹ and R² form a 5 membered heterocyclic ring

R⁴ is hydrogen or C¹-C⁶ alkyl

R³ is phenyl or substituted phenyl,

R⁵* is hydrogen or C¹-C⁶ alkyl,

The biradical R2*-R4* designate a bivalent linker group.

In some embodiments, B may for example be a protected nucleobase, R⁵* may be hydrogen, and R⁴ may be hydrogen or methyl.

The —Y—X— radical may be as described for the R⁴*-R²* radical as described herein under the description of LNA, such as may be selected from the group consisting of —CH₂—O—, —CH₂—CH₂—O—, CH(CH₃)—O—, —CH₂—S— and CH₂—NR′, wherein R′ is hydrogen of C₁-C₆ alkyl.

The invention also provides for the use of the LNA monomer of the invention for oligonucleotide synthesis.

The invention provides for a method of synthesising an LNA oligonucleotide said method comprising the steps of coupling the monomer of the invention to either an oligonucleotide synthesis support, or a preceding nucleotide. The method may use standard phosphoramidite synthesis protocols, although extended coupling times may be needed for the above coupling step. See for example the methodology use by Wan et al., NAR November 2014 (Advanced Publication), hereby incorporated by reference. Typically, the coupling is performed in the presence of an activator, such as 4,5 dicyanoimidazole or tetrazol. The coupling step may be followed by a oxidation or thiolation step. The invention provides for a oligonucleotide prepared by the method of the invention.

The invention provides for a stereoselective phosphorothioate LNA oligonucleotide, comprising at least one stereoselective phosphorothioate linkage between a LNA nucleoside and a subsequent (3′) nucleoside.

The invention provides for an oligonucleotide comprising at least one stereospecific phosphorothioate nucleotide pair wherein the phosphorothioate internucleoside linkage between the nucleotides pair is either in the Rp configuration or in the Rs configuration, and wherein at least one of the nucleosides of the nucleotide pair is a LNA nucleotide. Such as nucleotide pair is referred to as a LNA dinucleotide herein. In some embodiments both nucleosides of the nucleotide pair are LNA nucleotides. In some embodiments one of the nucleosides of the nucleotides pair is an LNA nucleoside and the other is a non-DNA nucleoside, such as a nucleoside analogue, such as a 2′substituted nucleoside. In some embodiments one of the nucleosides of the nucleotides pair has a 5′ LNA nucleoside. In some embodiments one of the nucleosides of the nucleotides pair has a 5′ LNA nucleoside and a 3′ nucleotide which is either LNA or a nucleoside other than LNA, such as a 2′ substituted nucleoside. In some embodiments one of the nucleosides of the nucleotides pair has a 5′ LNA nucleoside and a 3′ DNA nucleotide. In some embodiments one of the nucleosides of the nucleotides pair has a 3′ LNA nucleoside, and the other is a non-DNA nucleoside, such as a nucleoside analogue, such as a 2′substituted nucleoside. The oligonucleotide is at least 3 nucleotides in length, and may for example have a length of 7-30 nucleotides. The term oligonucleotide and oligomer are used interchangeably herein.

Typically, oligonucleotide phosphorothioates are synthesised as a random mixture of Rp and Sp phosphorothioate linkages. In the present invention, LNA phosphorothioate oligonucleotides are provided where at least one of the phosphorothioate linkages of the oligonucleotide is either Rp or Sp in at least 75%, such as at least 80%, or at least 85%, or at least 90% or at least 95%, or at least 97%, such as at least 98%, such as at least 99%, or all of the oligonucleotide molecules present in the oligonucleotide sample (i.e. a high proportion). Such oligonucleotides are referred as being stereoselective: They comprise at least one phosphorothioate linkage which is stereospecific. It is recognised that a stereoselective oligonucleotide may comprise s small amount of the alternative stereoisomer at any one position, for example Wan et al reports a 98% stereoselectivity for the gapmers reported in NAR, November 2014.

In some embodiments, the oligomer comprises at least two one nucleotide pair wherein the internucleoside linkage between the nucleotides pair is either in the Rp configuration or in the Sp configuration, and wherein at least one of the nucleosides of the nucleotide pair is a LNA nucleotide.

In some embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of the linkages in the oligomer are stereoselective phosphorothioate linkages. In some embodiments 5%, 10%, 15%, 20%, 25%, 30%, 35%, 40%, 45%, 50%, 55%, 60%, 65%, 70%, 75%, 80%, 85%, 90%, or 95% of the linkages in the oligomer are stereoselective phosphorothioate linkages. In some embodiments all of the phosphorothioate linkages in the oligomer are stereoselective phosphorothioate linkages. In some embodiments the all the internucleoside linkages of the oligomer are stereospecific phosphorothioate linkages. It should be recognised that stereospecificity refers to the incorporation of a high proportion of either the Rp or Sp internucleoside linkage at a defined internucleoside linkage.

The invention provides for an oligonucleotide of e.g. 6-30 nucleotides in length which comprises at least one stereospecific phosphorothioate linkage and at least one LNA nucleoside, wherein the oligomer does not comprise a region of more than 5 or 6 contiguous DNA units, or not more than 7 contiguous DNA units, or not more than 8 contiguous DNA units, or not more than 9 contiguous DNA units. The invention provides for a LNA mixmer or a LNA totalmer which comprises at least one stereospecific phosphorothioate linkage and at least one LNA nucleoside. In some embodiments the oligomer comprises one or more of the LNA dinucleotides referred to above.

The invention provides for a gapmer oligomer with at least one LNA nucleoside which is linked to the subsequent (3′) nucleoside via a stereospecific phosphorothioate linkage.

The invention provides a gapmer oligomer where the phosphorothioate internucleoside linkage between at least two adjacent LNA nucleosides is stereospecific, Sp or Rp. In some embodiments each wing of the gapmer comprises one or more stereospecific phosphorothioate internucleoside linkage between at least two adjacent LNA nucleosides.

In some embodiments, all the phosphorothioate internucleoside linkages between adjacent LNA nucleosides are stereospecific.

The invention further provides a conjugate comprising the oligomer according to the invention, which comprises at least one non-nucleotide or non-polynucleotide moiety (“conjugated moiety”) covalently attached to the oligomer of the invention. In some embodiments the conjugate of the invention is covalently attached to the oligomer via a biocleavable linker, which, for example may be a region of phosphodiester linked nucleotides, such as 1-5 PO linked DNA nucleosides (WO2014/076195, hereby incorporated by reference). Preferred conjugate groups include carbohydrate conjugates, such as GalNAc conjugates, such as trivalent GalNAc conjugates (e.g. see WO2014/118267, hereby incorporated by reference) or lipophilic conjugates, such as a sterol, e.g. cholesterol (WO2014/076195, hereby incorporated by reference)

The invention provides for pharmaceutical compositions comprising an oligomer or conjugate of the invention, and a pharmaceutically acceptable solvent (such as water or saline water), diluent, carrier, salt or adjuvant.

The invention further provides for an oligomer according to the invention, for use in medicine.

Pharmaceutical and other compositions comprising an oligomer of the invention are also provided. Further provided are methods of down-regulating the expression of a target nucleic acid, e.g. an RNA, such as a mRNA or microRNA in cells or tissues comprising contacting said cells or tissues, in vitro or in vivo, with an effective amount of one or more of the oligomers, conjugates or compositions of the invention.

Also disclosed are methods of treating an animal (a non-human animal or a human) suspected of having, or susceptible to, a disease or condition, associated with expression, or over-expression of a RNA by administering to the non-human animal or human a therapeutically or prophylactically effective amount of one or more of the oligomers, conjugates or pharmaceutical compositions of the invention.

The invention provides for methods of inhibiting (e.g., by down-regulating) the expression of a target nucleic acid in a cell or a tissue, the method comprising the step of contacting the cell or tissue, in vitro or in vivo, with an effective amount of one or more oligomers, conjugates, or pharmaceutical compositions thereof, to affect down-regulation of expression of a target nucleic acid.

Embodiments of the invention, which may be combined with the other embodiments of the invention described or claimed herein:
1. An LNA monomer of formula 1A or 1B:

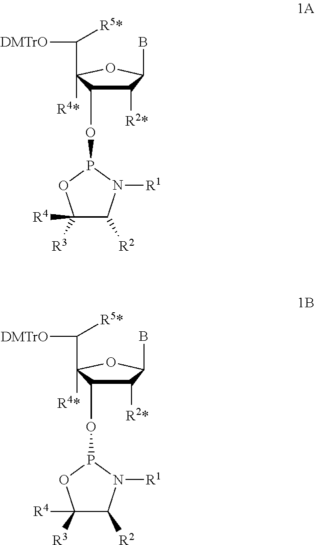

Wherein
B is selected from hydrogen, optionally substituted C₁₋₄-alkoxy, optionally substituted C₁₋₄-alkyl, optionally substituted C₁₋₄-acyloxy, nucleobases including naturally occurring and nucleobase analogues, DNA intercalators, photochemically active groups, thermochemically active groups, chelating groups, reporter groups, and ligands; preferably, B is a nucleobase or nucleobase analogue;

R¹ and R² form a 5 membered heterocyclic ring

R⁴ is hydrogen or C₁-C₆ alkyl

R³ is phenyl or substituted phenyl,

R⁵* is hydrogen or C₁-C₆ alkyl,

The biradical R²*-R⁴* designate a bivalent linker group.
2. The LNA monomer according to embodiment 1, of formula 2A or 2B

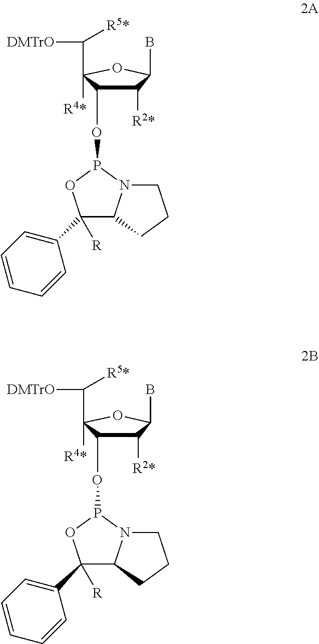

wherein R is hydrogen or C¹-C⁶ alkyl.
3. The LNA monomer according to embodiment 2, wherein R is H or methyl
4. The LNA monomer according to embodiment 1 or 2 of formula 3A or 3B

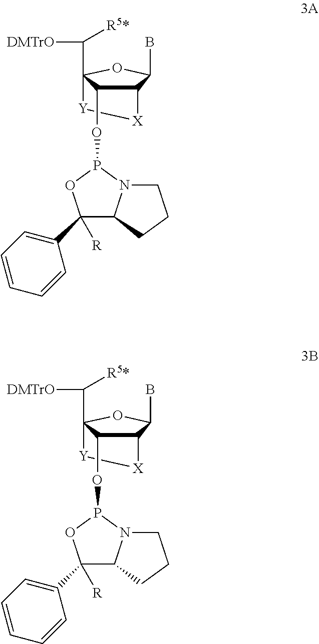

wherein Y—X is selected from the group consisting of —CH₂—O—, —CH₂—CH₂—O—, CH(CH₃)—O—, —CH₂—S— and CH₂—NR′, wherein R′ is hydrogen or C₁-C₆ alkyl, such as methyl.
5. The LNA monomer according to any one of the preceding embodiments wherein the biradical R⁴* and R^{2′} or —Y—X— together designate —CH₂—O- or —CH(CH₃)—O—.
6. The LNA monomer according to any one of the preceding embodiments of formula 4A or 4B

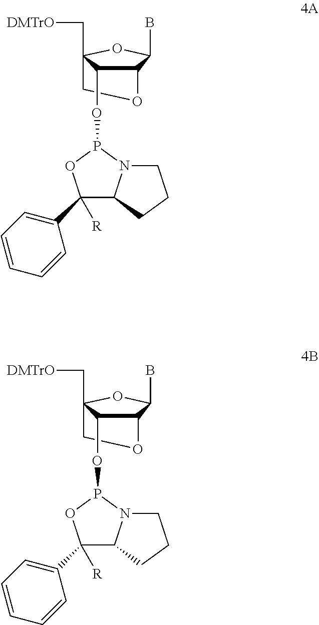

7. The LNA monomer according to any one of the preceding embodiments of formula 5A or 5B

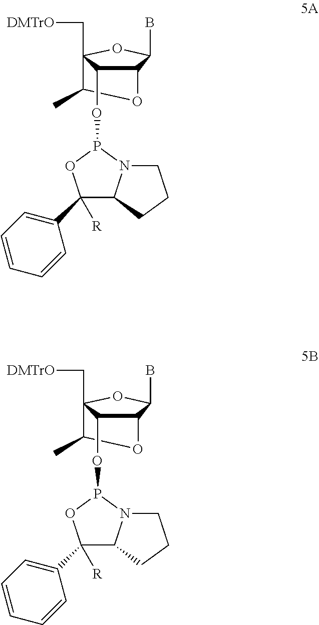

8. The LNA monomer according to any one of the preceding embodiments of formula 6A or 6B

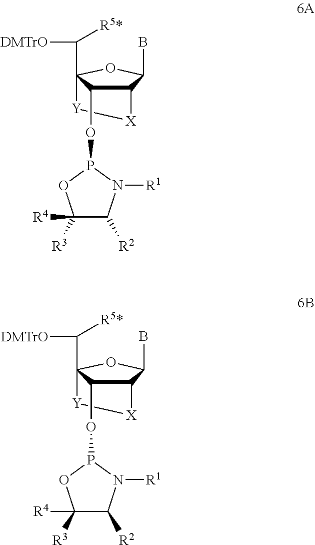

9. The LNA monomer according to any one of embodiments 1-8, wherein B is a nucleobase, such as a purine or pyrimidine nucleobase, such as a nucleobase selected from the group consisting of adenine, guanine, cytosine, 5′-methyl cytosine, thymidine, and uracil; or base protected nucleobase thereof.
10. The LNA monomer according to any one of embodiments 1-9, wherein R¹ and R² form a five membered heterocyclic ring, R⁴ is hydrogen, R³ is phenyl, the R4*-R2* biradical is selected from the group consisting of —CH₂—O—, —CH₂—CH₂—O—, CH(CH₃)—O—, —CH₂—S—, CH₂—NR′, wherein R′ is hydrogen of C₁-C₆ alkyl.
11. The LNA monomer according to any one of embodiments 1-10 wherein the R4*-R2* biradical is in the beta-D position.
12. The LNA monomer according to any one of embodiments 1-11 wherein the R4*-R2* biradical is —CH₂—O—.
13. The use of an LNA oligomer according to any one of embodiments 1-12 for the synthesis of an LNA oligonucleotide.
14. A method of synthesising an LNA oligonucleotide said method comprising the steps of coupling the monomer of any one of embodiments 1-12 to either an oligonucleotide synthesis support, or a preceding nucleotide.
15. The method according to embodiment 14, where in the coupling is in the presence of an activator, such as 4,5 dicyanoimidazole or tetrazol.
16. The method according to embodiment 14 or 15, wherein the coupling step is followed by a thiolation step.
17. An oligonucleotide produced by the method of any one of embodiments 13-16.
18. A stereoselective phosphorothioate LNA oligonucleotide, comprising at least one stereoselective phosphorothioate linkage between a LNA nucleoside and a subsequent (3′) nucleoside.
19. The stereoselective phosphorothioate LNA oligonucleotide of embodiment 18, which comprises at least one stereospecific phosphorothioate nucleotide pair wherein the internucleoside linkage between the nucleotides pair is either in the Rp configuration or in the Rs configuration, and wherein at least one of the nucleosides of the nucleotide pair is a LNA nucleotide.
20. The stereoselective phosphorothioate LNA oligonucleotide of embodiment 19, wherein the other nucleotide of the nucleotide pair is other than DNA, such as nucleoside analogue, such as a further LNA nucleoside or a 2′ substituted nucleoside.
21. A conjugate comprising the stereoselective phosphorothioate LNA oligonucleotide of any one of embodiments 18-20 covalently attached to a non-nucleoside moiety.
22. A pharmaceutical composition comprising the stereoselective phosphorothioate LNA oligonucleotide of any one of embodiments 18-20 or the conjugate of embodiment 20 and an a pharmaceutically acceptable solvent, (such as water or saline water), diluent, carrier, salt or adjuvant.
23. The stereoselective phosphorothioate LNA oligonucleotide of any one of embodiments 18-20 or the conjugate of embodiment 20, for use in medicine.

### EXAMPLES

### Sequences

The compounds used herein have the following nucleobase sequences:

| | |
|---|---|
| actgctttccactctg | SEQ ID NO 1 |
| tcatggctgcagct | SEQ ID NO 2 |
| gcattggtattca | SEQ ID NO 3 |
| cacattccttgctctg | SEQ ID NO 4 |
| gcaagcatcctgt | SEQ ID NO 5 |

### Example 1

Synthesis of DNA 3′-O-oxazaphospholidine monomers was performed as previously described (Oka et al., J. Am. Chem. Soc. 2008 130: 16031-16037, and Wan et al., NAR 2014, November, online publication).

### Synthesis of LNA 3′-O-oxazaphospholidine monomers

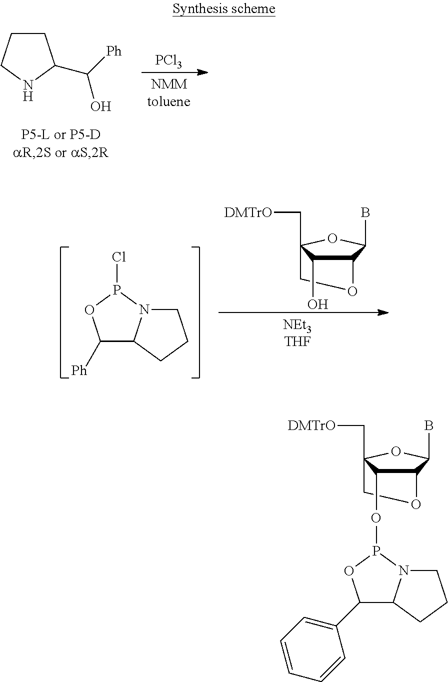

α-Phenyl-2-pyrrolidinemethanol (P5-L and P5-D) was synthesized as described in the literature (Oka et al., JACS, 2008, 16031-16037.)

### 3-OAP-LNA T

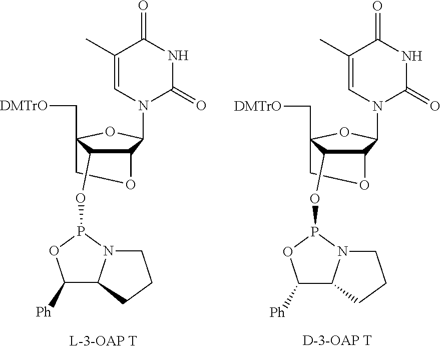

### Synthesis of L-3-OAP-LNA T

PCl₃ (735 μL, 6.30 mmol) was dissolved in toluene (7 mL), cooled to 0° C. (ice bath) and a solution of P5-L (1.12 g, 6.30 mmol) and NMM (1.38 mL, 12.6 mmol) in toluene (7 mL) was added dropwise. The reaction mixture was stirred at room temperature for 1h, and then cooled to −72° C. Precipitates were filtered under argon, washed with toluene (4 mL) and filtrate was concentrated at 40° C. and reduced pressure (Schlenk technique). The residue was dissolved in THF (8 mL) and used in the next step.

To a solution of 5′-ODMT-LNA-T (2.40 g, 4.20 mmol) in THF (16 mL), NEt₃ (4.10 mL, 29.4 mmol) was added. The reaction mixture was cooled to −74° C. and the solution of 2-chloro-1,3,2-oxazaphospholidine in THF was added dropwise. The reaction mixture was stirred for 4h at room temperature. EtOAc was added and the reaction mixture was extracted with sat. NaHCO₃ (2 times), brine, dried over Na₂SO₄ and evaporated. The residue was purified by column chromatography (eluent hexanes/EtOAc 30/70+NEt₃ 6%). Product was isolated as white foam 1.00 g (yield 30%). ¹H-NMR spectrum (400 MHz): (DMSO-d₆) δ: 11.53 (1H, s), 7.64 (1H, m), 7.46-7.41 (2H, m), 7.40-7.19 (12H, m), 6.92-6.83 (4H, m), 5.51 (1H, d, J=6.3 Hz), 5.49 (1H, s), 4.78 (1H, d, J=7.4 Hz), 4.37 (1H, s), 3.91 (1H, m), 3.76-3.67 (2H, m), 3.72 (3H, s), 3.71 (3H, s), 3.50 (1H, m), 3.41 (2H, s), 2.90 (1H, m), 1.60-1.46 (2H, m), 1.51 (3H, s), 1.15 (1H, m), 0.82 (1H, m). ³¹P-NMR spectrum (160 MHz): (DMSO-d₆) δ: 151.3. LCMS ESI (m/z): 776.2 [M−H]⁻.

### Synthesis of D-3-OAP-LNA T

PCl₃ (1.05 mL, 9.0 mmol) was dissolved in toluene (12 mL), cooled to 0° C. (ice bath) and a solution of P5-D (1.13 g, 12 mmol) and NMM (2.06 mL, 24 mmol) in toluene (12 mL) was added dropwise. The reaction mixture was stirred at room temperature for 1h, and then cooled to −72° C. Precipitates were filtered under argon, washed with toluene and filtrate was concentrated at 40° C. and reduced pressure (Schlenk technique). The residue was dissolved in THF (18 mL) and used in the next step.

To a solution of 5′-ODMT-LNA-T (3.44 g, 6.0 mmol) in THF (30 mL), NEt₃ (5.82 mL, 42 mmol) was added. The reaction mixture was cooled to −74° C. and the solution of 2-chloro-1,3,2-oxazaphospholidine in THF was added dropwise. The reaction mixture was stirred for 4h at room temperature. EtOAc was added and the reaction mixture was extracted with sat. NaHCO₃ (2 times), brine, dried over Na₂SO₄ and evaporated. The residue was purified by column chromatography (eluent hexanes/EtOAc 30/70+NEt₃ 6%). Product was isolated as a white foam 1.86 g (yield 36%), ¹H-NMR spectrum (400 MHz): (DMSO-d₆) δ: 11.55 (1H, s), 7.60 (1H, m), 7.46-7.41 (2H, m), 7.39-7.22 (12H, m), 6.91-6.84 (4H, m), 5.66 (1H, d, J=6.3 Hz), 5.51 (1H, s), 4.60 (1H, d, J=7.4 Hz), 4.41 (1H, s), 3.80-3.70 (3H, m), 3.72 (3H, s), 3.71 (3H, s), 3.48-3.37 (3H, m), 2.96 (1H, m), 1.61-1.43 (2H m), 1.51 (3H, s) 1.10 (1H, m), 0.80 (1H, m). ³¹P-NMR spectrum (160 MHz):(DMSO-d₆) δ: 152.5. LCMS ESI (m/z): 776.2 [M−H]⁻.

### 3-OAP-LNA MeC

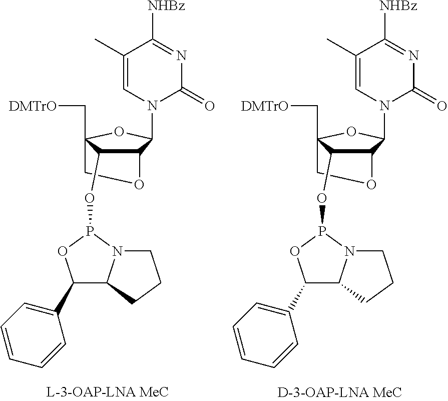

### Synthesis of L-3-OAP-LNA MeC

PCl₃ (110 μL, 1.25 mmol) was dissolved in toluene (3 mL), cooled to 0° C. (ice bath) and solution of P5-L (222 mg, 1.25 mmol) and NMM (275 μL, 2.5 mmol) in toluene (3 mL) was added dropwise. The reaction mixture was stirred at room temperature 45 min, and then cooled to −72° C. Precipitates were filtered under argon, washed with toluene and filtrate was concentrated at 40° C. at reduced pressure (Schlenk technique). The residue was dissolved in THF (5 mL) and used in the next step.

To solution of 5′-ODMT-LNA-C(338 mg, 0.50 mmol) in THF (2.5 mL) NEt₃ (485 μL, 3.6 mmol) was added. The reaction mixture cooled to −70° C. and the solution of phosphor 2-chloro-1,3,2-oxazaphospholidine was added dropwise. The reaction mixture was stirred for 1.45 h at room temperature. EtOAc (30 mL) was added and the reaction mixture was extracted with sat. NaHCO₃ (2×20 mL), brine (20 mL), dried over Na₂SO₄ and evaporated. The residue was purified by column chromatography (eluent EtOAc in hexanes from 20% to 30%+toluene 10%+NEt₃ 7%). Product isolated as white foam 228 mg (yield 47%). ¹H-NMR spectrum (400 MHz): (CD₃CN) δ: 13.3 (1H, br s), 8.41-8.25 (2H, m), 7.88 (1H, m), 7.59 (1H, m), 7.54-7.47 (4H, m), 7.41-7.19 (12H, m), 6.90-6.79 (4H, m), 5.62 (1H, m), 5.58 (1H, s), 4.79 (1H, d, J=7.5 Hz), 4.47 (1H, s), 3.93 (1H, m), 3.86 (1H, m), 3.75 (1H, m), 3.76 (3H, s), 3.75 (3H, s), 3.60-3.47 (3H, m), 2.99 (1H, m), 1.83 (3H, d, J=1.2 Hz), 1.65-1.51 (2H, m), 1.17 (1H, m), 0.89 (1H, m). ³¹P-NMR spectrum (160 MHz): (CD₃CN) δ: 153.4. LCMS ESI (m/z): 881.2 [M+H]⁺.

### Synthesis of D-3-OAP-LNA MeC

PCl₃ (1.10 mL, 12.3 mmol) was dissolved in toluene (10 mL), cooled to 0° C. (ice bath) and solution of P5-D (2.17 g, 12.3 mmol) and NMM (2.70 mL, 2.5 mmol) in toluene (10 mL) was added dropwise. The reaction mixture was stirred at room temperature 45 min, and then cooled to −72° C. Precipitates were filtered under argon, washed with toluene and filtrate was concentrated at 40° C. at reduced pressure (Schlenk technique). The residue was dissolved in THF (10 mL) and used in the next step.

To solution of 5′-ODMT-LNA-C(3.38 g, 5 mmol) in THF (20 mL) NEt₃ (4.85 mL, 35 mmol) was added. The reaction mixture cooled to −70° C. and the solution of phosphor 2-chloro-1,3,2-oxazaphospholidine was added dropwise. The reaction mixture was stirred for 1.45 h at room temperature. EtOAc was added and the reaction mixture was extracted with sat. NaHCO₃ (2×times), brine, dried over Na₂SO₄, and evaporated. The residue was purified by column chromatography (eluent EtOAc in hexanes from 20% to 30%+toluene 10%+NEt₃ 7%). Product was isolated as white foam 1.09 g (yield 23%). ¹H-NMR spectrum (400 MHz): (CD₃CN) δ: 12.8 (1H, br s), 8.34-8.24 (2H, m), 7.85 (1H, d, J=1.2 Hz), 7.57 (1H, m), 7.53-7.45 (4H, m), 7.41-7.22 (12H, m), 6.89-6.84 (4H, m), 5.72 (1H, d, J=6.5 Hz), 5.59 (1H, s), 4.62 (1H, d, J=8.0 Hz), 4.52 (1H, s), 3.82 (2H, dd, J=24.4 8.2 Hz), 3.77 (1H, m), 3.76 (3H, s), 3.75 (3H, s), 3.51 (2H, s), 3.46 (1H, m), 3.05 (1H, m), 1.81 (3H, s), 1.65-1.47 (2H, m), 1.12 (1H, m), 0.85 (1H, m). ³¹P-NMR spectrum (160 MHz): (CD₃CN) δ: 153.5. LCMS ESI (m/z): 881.2 [M+H]⁺.

### 3-OAP-LNA A

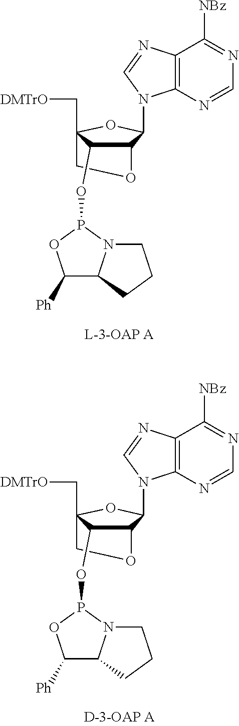

### Synthesis of L-3-OAP-LNA A

PCl₃ (184 μL, 2.1 mmol) was dissolved in toluene (5 mL), cooled to 0° C. (ice bath) and a solution of P5-L (373 mg, 2.10 mmol) and NMM (463 μL, 4.20 mmol) in toluene (5 mL) was added dropwise. The reaction mixture was stirred at room temperature for 45 min, and then cooled to −72° C. Precipitates was filtered under argon, washed with toluene (4 mL) and filtrate was concentrated at 40° C. at reduce pressure (Schlenk technique). The residue was dissolved in THF (5 mL) and used in the next step.

To a solution of 5′-ODMT-LNA-A (960 mg, 1.40 mmol) in THF (7 mL) NEt₃ (1.36 mL, 9.80 mmol) was added. The reaction mixture cooled to −70° C. and the solution of phosphor 2-chloro-1,3,2-oxazaphospholidine was added dropwise. The reaction mixture was stirred for 4 h at room temperature. EtOAc (50 mL) was added and the reaction mixture was extracted with sat. NaHCO₃ (2×30 mL), brine (30 mL), dried over Na₂SO₄ and evaporated. The residue was purified by column chromatography (eluent hexanes/EtOAc 30/70+NEt₃ 6-7%). Product isolated as white foam 455 mg (yield 35%). ¹H-NMR spectrum (400 MHz): (DMSO-d₆) δ: 11.33 (1H, s), 8.76 (1H, s), 8.53 (1H, s), 8.11-8.02 (2H, m), 7.66 (1H, m), 7.60-7.53 (2H, m), 7.44-7.38 (2H, m), 7.35-7.18 (10H, m), 7.05-6.99 (2H, m), 6.89-6.82 (4H, m), 6.21 (1H, s), 5.27 (1H, d, J=6.6 Hz), 5.19 (1H, d, J=7.9 Hz), 4.81 (1H, s), 3.93 (2H, dd, J=29.0 8.2 Hz), 3.77 (1H, m), 3.71 (6H, s), 3.51-3.35 (3H, m), 2.70 (1H, m), 1.56-1.34 (2H, m), 1.10 (1H, m), 0.73 (1H, m). ³¹P-NMR spectrum (160 MHz): (DMSO-d₆) δ: 149.9. LCMS ESI (m/z): 891.1 [M+H]⁺.

### Synthesis of D-3-OAP-LNA A

PCl₃ 0.84 mL, 9.63 mmol) was dissolved in toluene (12 mL), cooled to 0° C. (ice bath) and a solution of P5-D (1.70 g, 9.63 mmol) and NMM (2.12 mL, 19.3 mmol) in toluene (12 mL) was added dropwise. The reaction mixture was stirred at room temperature for 45 min, and then cooled to −72° C. Precipitates was filtered under argon, washed with toluene and filtrate was concentrated at 40° C. at reduce pressure (Schlenk technique). The residue was dissolved in THF (12 mL) and used in the next step.

To a solution of 5′-ODMT-LNA-A (3.77, 5.50 mmol) in THF (20 mL) NEt₃ (5.30 mL, 38.5 mmol) was added. The reaction mixture cooled to −70° C. and the solution of phosphor 2-chloro-1,3,2-oxazaphospholidine was added dropwise. The reaction mixture was stirred for 4 h at room temperature. EtOAc was added and the reaction mixture was extracted with sat. NaHCO₃, brine, dried over Na₂SO₄ and evaporated. The residue was purified by column chromatography (eluent hexanes/EtOAc 30/70+NEt₃ 6-7%). Product was isolated as a white foam 1.86 g (yield 36%). ¹H-NMR spectrum (400 MHz): (DMSO-d₆) δ: 11.28 (1H, s), 8.78 (1H, s), 8.54 (1H, s), 8.09-8.04 (2H, m), 7.67 (1H, m), 7.60-7.54 (2H, m), 7.42-7.15 (14H, m), 6.89-6.82 (4H, m), 6.21 (1H, s), 5.58 (1H, d, J=6.7 Hz), 5.02 (1H, d, J=8.1 Hz), 4.89 (1H, s), 3.96 (2H, dd, J=35.4 8.2 Hz), 3.71 (3H, s), 3.70 (3H, s), 3.53-3.33 (4H, m), 2.90 (1H, m), 1.54-1.37 (2H, m), 0.98 (1H, m), 0.71 (1H, m). ³¹P-NMR spectrum (160 MHz): (DMSO-d₆) δ: 150.6, 150.5 (2%), 150.4. LCMS ESI (m/z): 891.1 [M+H]⁺.

### 3-OAP-LNA G

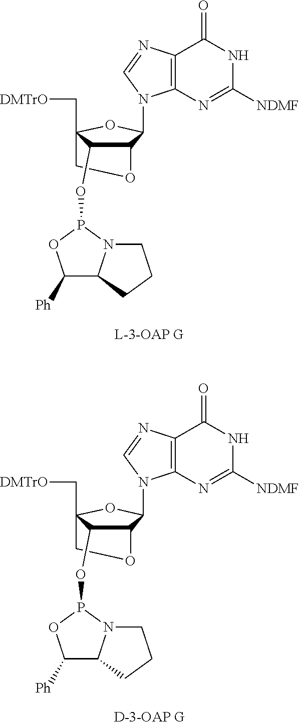

### Synthesis of D-3-OAP-LNA G

PCl₃ (1.09 mL, 12.4 mmol) was dissolved in toluene (12.5 mL), cooled to 0° C. (ice bath) and a solution of P5-D (2.20 g, 12.4 mmol) and NMM (2.73 mL, 27.8 mmol) in toluene (12.5 mL) was added dropwise. The reaction mixture was stirred at room temperature for 45 min, and then cooled to −72° C. Precipitates was filtered under argon, washed with toluene and filtrate was concentrated at 40° C. at reduce pressure (Schlenk technique). The residue was dissolved in THF (19 mL) and used in the next step.

Before synthesis 5′-ODMT-LNA-G was co evaporated with toluene and then with pyridine (order is essential). To solution of 5′-ODMT-LNA-G (3.26 g, 5.0 mmol) in THF (15 mL) and Pyridine (8 mL), NEt₃ (4.85 mL, 35.0 mmol) was added. The reaction mixture cooled to −70° C. and the solution of phosphor 2-chloro-1,3,2-oxazaphospholidine was added dropwise. The reaction mixture was stirred for 2.5 h at room temperature. EtOAc was added and the reaction mixture was extracted with sat. NaHCO₃, brine, dried over Na₂SO₄ and evaporated.

The residue was purified by column chromatography (eluent THF in EtOAc from 10% to 20%+NEt₃ 6%). Product isolated as white foam 1.49 g (yield 33%). ¹H-NMR spectrum (400 MHz): (DMSO-d₆) δ: 11.42 (1H, s), 8.56 (1H, s), 7.95 (1H, s), 7.49-7.38 (2H, m), 7.36-7.16 (12H, m), 6.90-6.83 (4H, m), 5.96 (1H, s), 5.58 (1H, d, J=6.7 Hz), 4.99 (1H, d, J=8.2 Hz), 4.76 (1H, s), 3.96-3.85 (2H, m), 3.72 (6H, s), 3.62-3.54 (1H, m), 3.45 (2H, s), 3.40-3.33 (1H, m), 3.08 (3H, s), 2.99 (3H, s), 2.93-2.84 (1H, m), 1.53-1.39 (2H, m), 1.06-0.97 (1H, m), 0.79-0.63 (1H, m). ³¹P-NMR spectrum (160 MHz): (DMSO-d₆) δ: 151.6. LCMS ESI (m/z): 858.2 [M+H]⁺.

### Synthesis of L-3-OAP-LNA G

PCl₃ (1.00 mL, 11.4 mmol) was dissolved in toluene (10 mL), cooled to 0° C. (ice bath) and a solution of P5-L (2.02 g, 11.4 mmol) and NMM (2.50 mL, 22.7 mmol) in toluene (10 mL) was added dropwise. The reaction mixture was stirred at room temperature for 45 min, and then cooled to −72° C. Precipitates was filtered under argon, washed with toluene and filtrate was concentrated at 40° C. at reduce pressure (Schlenk technique). The residue was dissolved in THF (7 mL) and used in the next step.

Before synthesis 5′-ODMT-LNA-G was co evaporated with toluene and then with pyridine (order is essential). To a solution of 5′-ODMT-LNA-G (2.86 g, 4.54 mmol) in THF (20 mL) and Pyridine (12 mL), NEt₃ (4.40 mL, 31.8 mmol) was added. The reaction mixture cooled to −70° C. and the solution of phosphor 2-chloro-1,3,2-oxazaphospholidine was added dropwise. The reaction mixture was stirred for 2.5 h at room temperature. EtOAc was added and the reaction mixture was extracted with sat. NaHCO₃, brine, dried over Na₂SO₄ and evaporated. The residue was purified by column chromatography (eluent THF in EtOAc from 10% to 20%+NEt₃ 6%). Product isolated as white foam 1.44 g (yield 34%). ¹H-NMR (400 mHz, DMSO-d₆): δ:11.44 (1H, s), 8.42 (1H, s), 7.94 (1H, s), 7.44-7.38 (2H, m), 7.34-7.23 (10H, m), 7.03-6.98 (2H, m), 5.94 (1H, s), 5.17 (1H, d, J=6.5 Hz), 5.07 (1H, d, J=7.8 Hz), 4.68 (1H, s), 3.88 (1H, d, J=8.2 Hz), 3.84 (1H, d, J=8.2 Hz), 3.73 (3H, s), 3.72 (3H, s), 3.68 (1H, m), 3.46-3.36 (3H, m), 3.05 (3H, s), 2.95 (3H, s), 2.77 (1H, m), 1.55-1.38 (2H, m), 1.07 (1H, m), 0.75 (1H, m). ³¹P-NMR (160 MHz, DMSO-d₆): δ:148.4. LCMS ESI(m/z): 858.5 [M+H]⁺; 856.5 [M−H]⁻

### Generic Synthesis Description

Synthesis of phosphor 2-chloro-1,3,2-oxazaphospholidine: PCl₃ (1 eq) was dissolved in toluene, cooled to 0° C. (ice bath) and a solution of P5-L (1 eq) and NMM (2.1 eq) in toluene was added dropwise. The reaction mixture was stirred at room temperature, and then cooled to −72° C. Precipitates was filtered under argon, washed with toluene and filtrate was concentrated at 40° C. at reduce pressure (Schlenk technique). The residue was dissolved in THF and used in the next step.

To a solution of 5′-ODMT-LNA nucleoside (1 eq) in THF (and Pyridine in case of G nucleoside), NEt₃ (7 eq) was added. The reaction mixture cooled to −70° C. and the solution of phosphor 2-chloro-1,3,2-oxazaphospholidine (2.5 eq) was added dropwise. The reaction mixture was stirred for at room temperature. EtOAc was added and the reaction mixture was extracted with sat. NaHCO₃, brine, dried over Na₂SO₄ and evaporated. The residue was purified by column chromatography.

### Structure Figures of the LNA Monomers

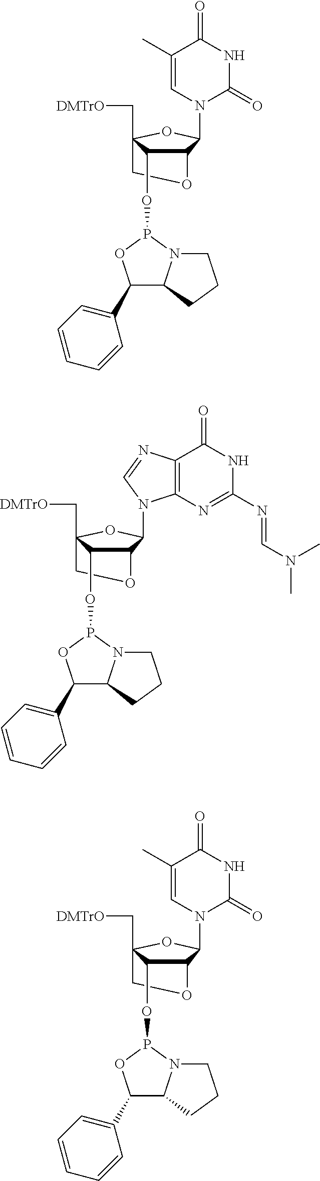

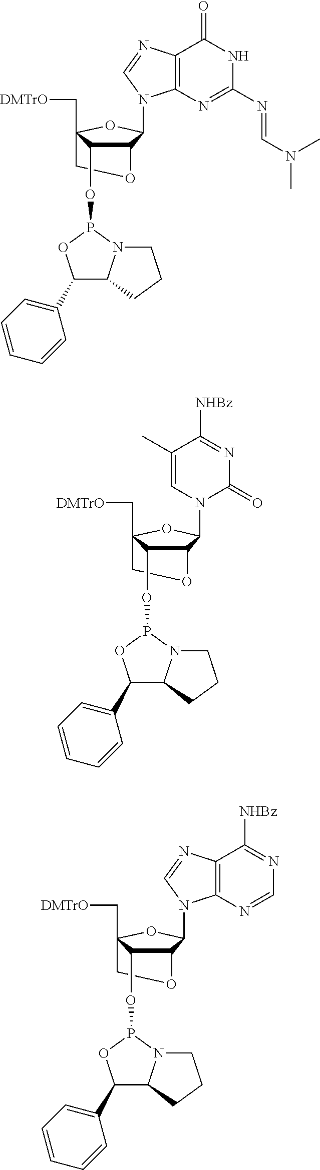

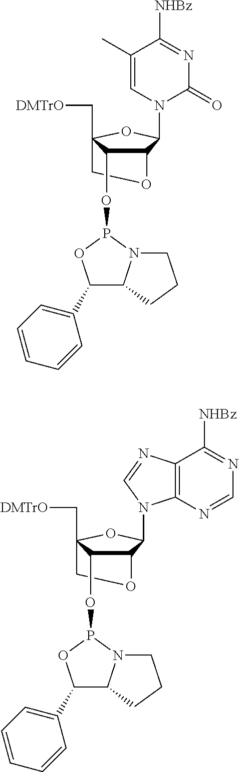

The following LNA-oxazaphospholine LNA monomers were synthesized using the method disclosed in Oka et al., J. Am. Chem. Soc. 2008; 16031-16037:

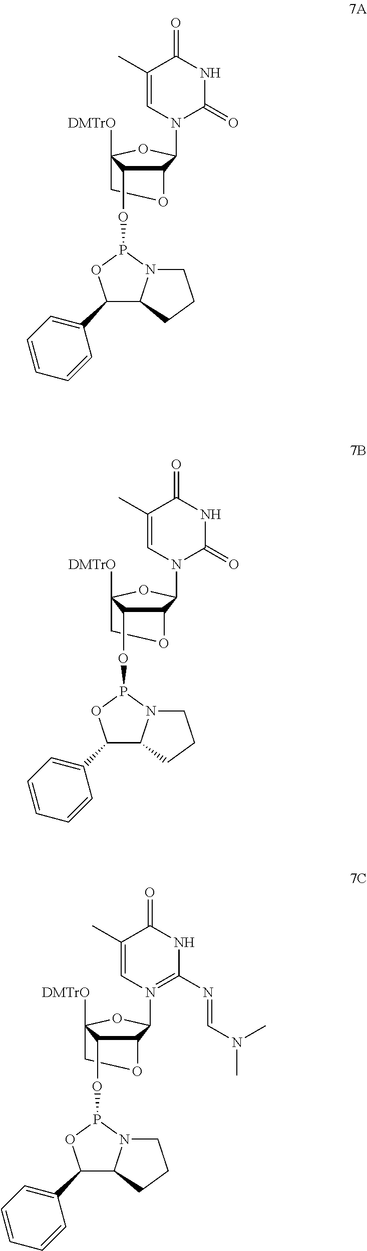

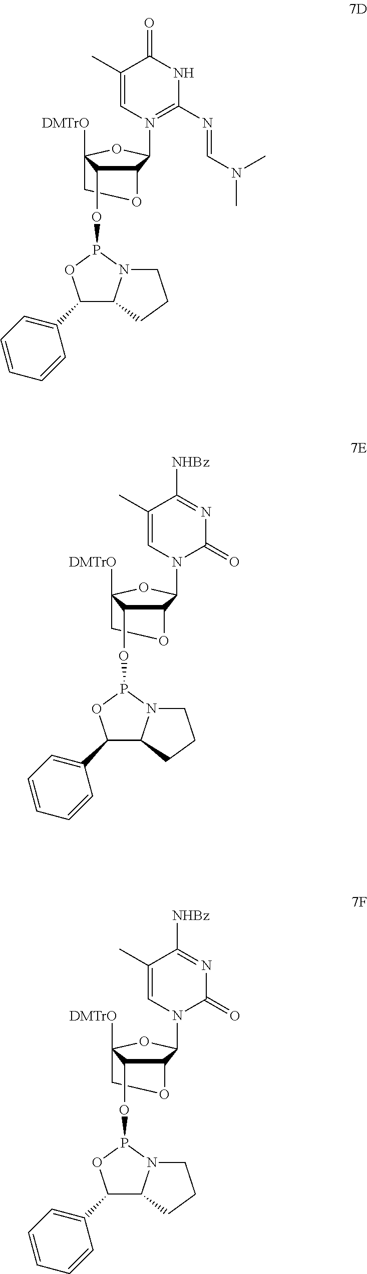

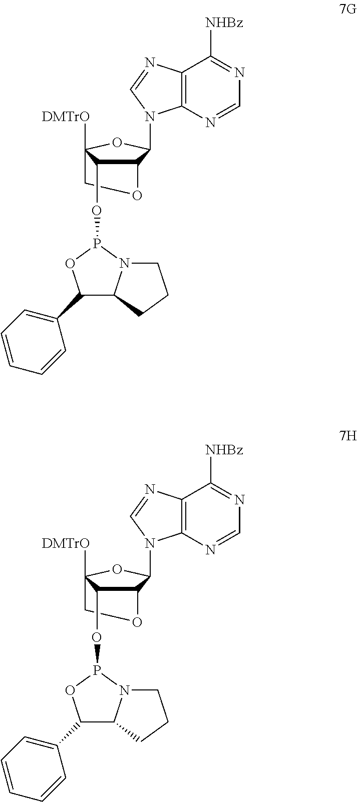

The above LNA monomers were used in oligonucleotide synthesis and shown to give stereocontrolled phosphoramidite LNA oligonucleotides as determined by HPLC.

### Example 2

The following LNA oligonucleotides targeting Myd88 are synthesized.

(Parent #1)
(SEQ ID NO 1)
Aₓ^{m}CₓTₓgₓcₓtₓtₓtₓcₓcₓaₓcₓtₓ^{m}CₓTₓG
(Parent #1)
Aₓ^{m}CₓTₓgₓcₓtₓtₓtₓcₓcₓaₓcₓtₓ^{m}CₓTₓG
(Comp #2)
Aₓ^{m}CₓTₓgₓcₛtₓtₓtₓcₓcₓaₓcₓtₓ^{m}CₓTₓG
(Comp #3)
Aₓ^{m}CₓTₓgₓcₓtₓtₓtₓcₛcₓaₓcₓtₓ^{m}CₓTₓG
(Comp #4)
Aₓ^{m}CₓTₓgₓcₓtₓtₓtₓcₓcₓaₓcₛtₓ^{m}CₓTₓG
(Comp #5)
Aₓ^{m}CₓTₓgₓcᵣtₓtₓtₓcₓcₓaₓcₓtₓ^{m}CₓTₓG
(Comp #6)
Aₓ^{m}CₓTₓgₓcₓtₓtₓtₓcₓcₓaₓcₓtₓ^{m}CₓTₓG
(Co^{m}p #7)
Aₓ^{m}CₓTₓgₓcₓtₓtₓtₓcₓcₓaₓcᵣtₓ^{m}CₓTₓG
(Comp #8)
Aₓ^{m}CₓTₓgₓcₛtₓtₓtₓcₓcₓaₓcₓtₓ^{m}CₓTₓG
(Comp #9)
Aₓ^{m}CₓTₓgₓcₛtₓtₓtₓcₓcₓaₓcₛtₓ^{m}CₓTₓG
(Comp #10)
Aₓ^{m}CₓTₓgₓcₛtₓtₓtₓcₓcₓaₓcₛtₓ^{m}CₓTₓG
(Comp #11)
Aₓ^{m}CₓTₓgₓcₓtₓtₓtₓcₛcₓaₓcₛtₓ^{m}CₓTₓG
(Comp #12)
Aₓ^{m}CₓTₓgₓcᵣtₓtₓtₓcₓcₓaₓcₓtₓ^{m}CₓTₓG
(Comp #13)
Aₓ^{m}CₓTₓgₓcᵣtₓtₓtₓcₓcₓaₓcᵣtₓ^{m}CₓTₓG
(Comp #14)
Aₓ^{m}CₓTₓgₓcᵣtₓtₓtₓcₓcₓaₓcᵣtₓ^{m}CₓTₓG
(Comp #15)
Aₓ^{m}CₓTₓgₓcₓtₓtₓtₓcₓcₓaₓcᵣtₓ^{m}CₓTₓG
(Comp #16)
Aₓ^{m}CₓTₓgₓcₓtₓtₓtₓcₓcₓaₓcₛtₓ^{m}CₓTₓG
(Comp #17)
Aₓ^{m}CₓTₓgₓcₓtₓtₓtₓcₛcₓaₓcᵣtₓ^{m}CₓTₓG
(Comp #18)
Aₓ^{m}CₓTₓgₓcₛtₓtₓtₓcₓcₓaₓcₓtₓ^{m}CₓTₓG
(Comp #19)
Aₓ^{m}CₓTₓgₓcₛtₓtₓtₓcₓcₓaₓcᵣtₓ^{m}CₓTₓG
(Comp #20)
Aₓ^{m}CₓTₓgₓcₛtₓtₓtₓcₓcₓaₓcᵣtₓ^{m}CₓTₓG
(Comp #21)
Aₓ^{m}CₓTₓgₓcₛtₓtₓtₓcₓcₓaₓcₛtₓ^{m}CₓTₓG
(Comp #22)
Aₓ^{m}CₓTₓgₓcₛtₓtₓtₓcₛcₓaₓcᵣtₓ^{m}CₓTₓG
(Comp #23)
Aₓ^{m}CₓTₓgₓcᵣtₓtₓtₓcₓcₓaₓcₓtₓ^{m}CₓTₓG
(Comp #24)
Aₓ^{m}CₓTₓgₓcᵣtₓtₓtₓcₓcₓaₓcₛtₓ^{m}CₓTₓG
(Comp #25)
Aₓ^{m}CₓTₓgₓcᵣtₓtₓtₓcₓcₓaₓcₛtₓ^{m}CₓTₓG
(Comp #26)
Aₓ^{m}CₓTₓgₓcᵣtₓtₓtₓcₓcₓaₓcᵣtₓ^{m}CₓTₓG
(Comp #27)
Aₓ^{m}CₓTₓgₓcᵣtₓtₓtₓcₓcₓaₓcₛtₓ^{m}CₓTₓG

Capital letters are beta-D-oxy LNA nucleosides, small letters are DNA nucleosides Subscript x=randomly incorporated phosphorothioate linkage from a racemic mixture of Rp and Sp monomers.

Subscript s=stereocontrolled phosphoramidite linkage from a Sp monomer

Subscript r=stereocontrolled phosphoramidite linkage from a Rp monomer

Superscript m preceding a capital C represents 5-methyl cytosine LNA nucleoside

### Example 3

Parent compound #1 has been determined as a hepatotoxic in mice. Compounds #1-27# are evaluated for their hepatotoxicity in an in vivo assay: 5 NMRI female mice per group are used, 15 mg/kg of compound are administered to each mouse on days 0, 3, 7, 10 and 14, and sacrificed on day 16. Serum ALT is measured. Hepatotoxicity may also be measured as described in EP 1 984 381, example 41 with the exception that NMRI mice are used, or using an in vitro hepatocyte toxicity assay.

### Example 4

The following LNA oligonucleotides identified as toxic in Seth et al J. Med. Chem 2009, 52, 10-13 are synthesized.

(Parent #28)
(SEQ ID NO 2)
Tₓ^{m}Cₓaₓtₓgₓgₓcₓtₓgₓcₓaₓgₓ^{m}CₓT
(Comp #29)
Tₓ^{m}Cₓaₓtₛgₓgₓcₓtₓgₓcₓaₓgₓ^{m}CₓT
(Comp #31)
Tₓ^{m}Cₓaₓtₓgₓgₛcₓtₓgₓcₓaₓgₓ^{m}CₓT
(Comp #32)
Tₓ^{m}Cₓaₓtₓgₓgₓcₓtₓgₓcₛaₓgₓ^{m}CₓT
(Comp #33)
Tₓ^{m}Cₓaₓtₛgₓgₛcₓtₓgₓcₓaₓgₓ^{m}CₓT
(Comp #34)
Tₓ^{m}Cₓaₓtₛgₓgₓcₓtₓgₓcₛaₓgₓ^{m}CₓT
(Comp #35)
Tₓ^{m}Cₓaₓtₛgₓgₛcₓtₓgₓcₛaₓgₓ^{m}CₓT
(Comp #36)
Tₓ^{m}Cₓaₓtₓgₓgₛcₓtₓgₓcₛaₓgₓ^{m}CₓT
(Comp #37)
Tₓ^{m}Cₓaₓtₛgₓgᵣcₓtₓgₓcₓaₓgₓ^{m}CₓT
(Comp #38)
Tₓ^{m}Cₓaₓtₛgₓgₓcₓtₓgₓcᵣaₓgₓ^{m}CₓT
(Comp #39)
Tₓ^{m}Cₓaₓtₛgₓgᵣcₓtₓgₓcᵣaₓgₓ^{m}CₓT
(Comp #40)
Tₓ^{m}Cₓaₓtₓgₓgₛcₓtₓgₓcᵣaₓgₓ^{m}CₓT
(Comp #41)
Tₓ^{m}Cₓaₓtₛgₓgᵣcₓtₓgₓcₛaₓgₓ^{m}CₓT
(Comp #42)
Tₓ^{m}Cₓaₓtₛgₓgₛcₓtₓgₓcᵣaₓgₓ^{m}CₓT
(Comp #43)
Tₓ^{m}Cₓaₓtᵣgₓgₓcₓtₓgₓcₓaₓgₓ^{m}CₓT
(Comp #44)
Tₓ^{m}Cₓaₓtₓgₓgᵣcₓtₓgₓcₓaₓgₓ^{m}CₓT
(Comp #45)
Tₓ^{m}Cₓaₓtₓgₓgₓcₓtₓgₓcᵣaₓgₓ^{m}CₓT
(Comp #46)
Tₓ^{m}Cₓaₓtᵣgₓgᵣcₓtₓgₓcₓaₓgₓ^{m}CₓT
(Comp #47)
Tₓ^{m}Cₓaₓtᵣgₓgₓcₓtₓgₓcᵣaₓgₓ^{m}CₓT
(Comp #48)
Tₓ^{m}Cₓaₓtᵣgₓgᵣcₓtₓgₓcᵣaₓgₓ^{m}CₓT
(Comp #49)
Tₓ^{m}Cₓaₓtₓgₓgᵣcₓtₓgₓcᵣaₓgₓ^{m}CₓT
(Comp #50)
Tₓ^{m}Cₓaₓtᵣgₓgₛcₓtₓgₓcₓaₓgₓ^{m}CₓT
(Comp #51)
Tₓ^{m}Cₓaₓtᵣgₓgₓcₓtₓgₓcₛaₓgₓ^{m}CₓT
(Comp #52)
Tₓ^{m}Cₓaₓtᵣgₓgₛcₓtₓgₓcₛaₓgₓ^{m}CₓT
(Comp #53)
Tₓ^{m}Cₓaₓtₓgₓgᵣcₓtₓgₓcₛaₓgₓ^{m}CₓT
(Comp #54)
Tₓ^{m}Cₓaₓtᵣgₓgₛcₓtₓgₓcᵣaₓgₓ^{m}CₓT
(Comp #55)
Tₓ^{m}Cₓaₓtᵣgₓgᵣcₓtₓgₓcₛaₓgₓ^{m}CₓT

Capital letters are beta-D-oxy LNA nucleosides, small letters are DNA nucleosides

Subscript x=randomly incorporated phosphorothioate linkage from a racemic mixture of Rp and Sp monomers.

Subscript s=stereocontrolled phosphoramidite linkage from a Sp monomer

Subscript r=stereocontrolled phosphoramidite linkage from a Rp monomer

Superscript m preceding a capital C represents 5-methyl cytosine LNA nucleoside

### Example 5

Parent compound #28 has been determined as a hepatotoxic in mice. Compounds #28-27# are evaluated for their hepatotoxicity in an in vivo assay: 5 NMRI female mice per group are used, 15 mg/kg of compound are administered to each mouse on days 0, 3, 7, 10 and 14, and sacrificed on day 16. Serum ALT is measured. Hepatotoxicity may also be measured as described in EP 1 984 381, example 41 with the exception that NMRI mice are used, or using an in vitro hepatocyte toxicity assay.

### Example 6. Tolerance and Tissue Content of Compound Libraries with 3 Fixed PS Internucleoside Linkages In Vivo

C57BL6/J mice (5 animals/gr) were injected iv on day 0 with a single dose saline or 30 mg/kg LNA-antisense oligonucleotide in saline (seq ID #1, 10, or 14) and sacrificed on day 8.

Serum was collected and ALT was measured for all groups. The oligonucleotide content was measured in the LNA dosed groups using ELISA method.

### Conclusions:

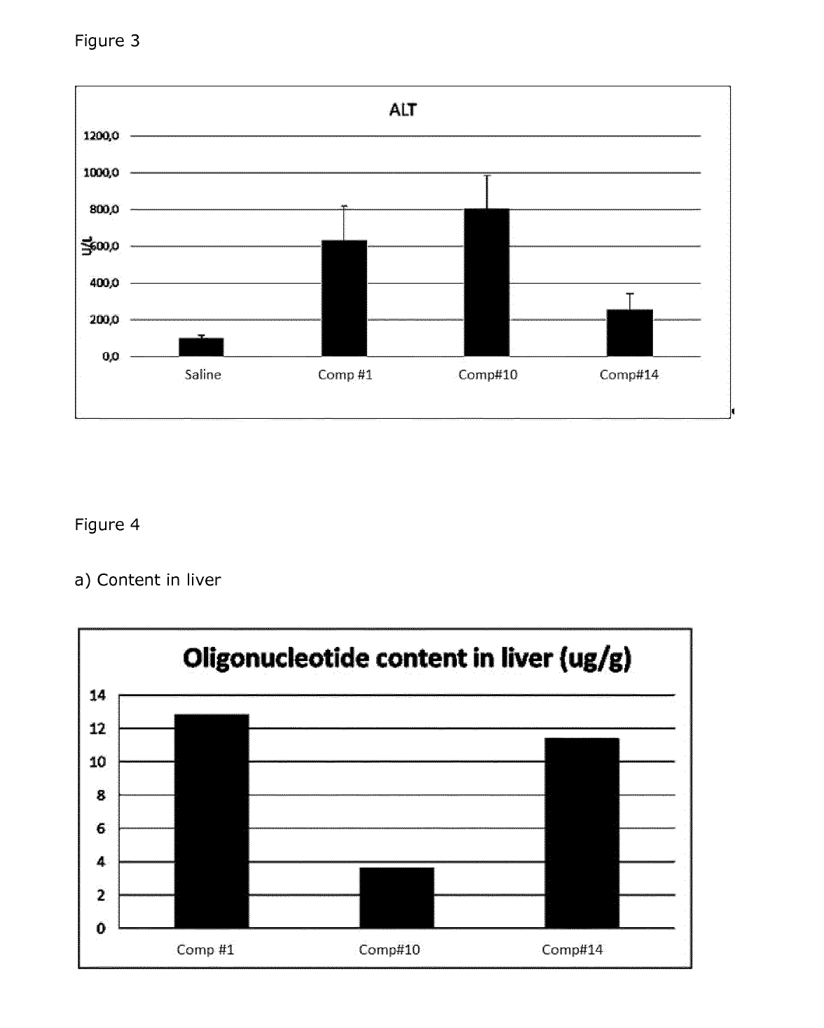

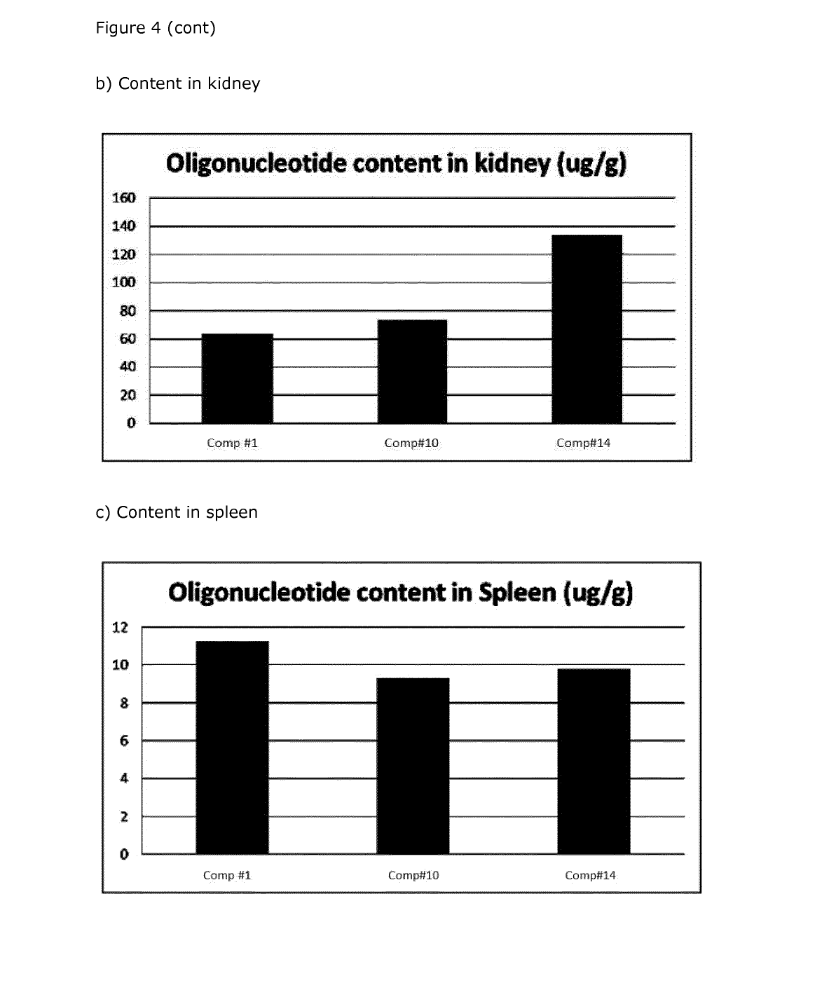

The hepatotoxic potential (ALT) for the subgroups of LNA oligonucleotides where 3 phosphorothioate internucleoside linkages are fixed in either S (Comp #10) or R (Comp #14) configuration was compared to the ALT for parent mixture of diastereoisomers (Comp #1) with all internucleoside linkages as mixtures of R and S configuration. It is seen (FIG. 3) that for one subgroup (Comp #14) the ALT readout is significantly lower than for the parent mixture (Comp #1) and for the other subgroup of compounds (Comp #10) ALT reading is similar to parent. Moreover, the liver uptake profile (FIG. 4a) show that the subgroup of LNA oligonucleotides with low ALT readout (Comp #14) is taken up into the liver to the same extend as the parent LNA mixture (Comp #1) whereas the other subgroup (Comp #10) with ALT comparable to the parent mixture (Comp #1) is taken up less into the liver. Kidney uptake (FIG. 4b) is similar for parent LNA (Comp #1) and one subgroup (Comp #10) and higher for the other subgroup of LNA oligonucleotides (Comp #14). Uptake into the spleen is similar for all 3 groups of compounds (FIG. 4c). Generally it is seen that fixing the stereochemistry in some positions and thereby generating a subgroup of LNA oligonucleotides induces differences for properties such as uptake and hepatotoxic potential compared to the parent mixture of LNA oligonucleotides.

### Materials and Methods:

### Experimental Design:

TABLE 2

| Groups | Compounds | Day 1 | Day 2 | Day 3 | Day 5 | Day 8 |
|---|---|---|---|---|---|---|
| 1 | Saline | Body | Body | Body | Body | Blood |
| | | weight | weight | weight | weight | Body |
| | | Dosing | | | | weight |
| | | | | | | Termi- |
| | | | | | | nation |
| 2 | Comp #1 | Body | Body | Body | Body | Blood |
| | 30 mg/kg | weight | weight | weight | weight | Body |
| | | Dosing | | | | weight |
| | | | | | | Termi- |
| | | | | | | nation |
| 3 | Comp #10 | Body | Body | Body | Body | Blood |
| | 30 mg/kg | weight | weight | weight | weight | Body |
| | | Dosing | | | | weight |
| | | | | | | Termi- |
| | | | | | | nation |
| 4 | Comp #14 | Body | Body | Body | Body | Blood |
| | 30 mg/kg | weight | weight | weight | weight | Body |
| | | Dosing | | | | weight |
| | | | | | | Termi- |
| | | | | | | nation |

### Dose Administration.

C57BL/6JBom female animals, app. 20 g at arrival, were dosed with 10 ml per kg BW (according to day 0 bodyweight) i.v. of the compound formulated in saline or saline alone according to Table 2.

### Sampling of Liver and Kidney Tissue.

The animals were anaesthetised with 70% CO₂-30% O₂ and sacrificed by cervical dislocation according to Table 2. One half of liver and one kidney was frozen and used for tissue analysis.

Oligonucleotide content in liver and kidney was measured by sandwich ELISA method. ALT levels were measured

### Example 7 RNase H Activity of Chirally Defined Phosphorothioate LNA Gapmers

The parent compound used, 3833 was used:

(SEQ ID NO 3)
5′-Gₛ^{m}CₛaₛtₛtₛgₛgₛtₛaₛtₛTₛ^{m}CₛA-3′

Wherein capital letters represent beta-D-oxy-LNA nucleosides, lower case letters represent DNA nucleosides, subscript s represents random s or r phosphorothioate linkages (not chirally defined during oligonucleotide synthesis), and superscript m prior to C represents 5-methyl cytosine LNA nucleoside.

A range of fully chirally defined variants of 3833 were designed with uniques patterns of R and S at each of the 12 internucleoside positions, as illustrated by either an S or an R. The RNaseH recruitment activity and cleavage pattern was determined using human RNase H, and compared to the parent compound 3833 (chirality mix) as well as a fully phosphodiester linked variant of 3833 (full PO), and a 3833 compound which comprises of phosphodiester linkages within the central DNA gap region and random PS linkages in the LNA flank (PO gap).

### Compounds:

| Oligo | Chirality of nucleo base linkages | | | | | | | | | | | |
|---|---|---|---|---|---|---|---|---|---|---|---|---|
| no. | 1 | 2 | 3 | 4 | 5 | 6 | 7 | 8 | 9 | 10 | 11 | 12 |
| 16614 | S | R | S | R | R | S | R | S | S | S | R | R |
| 16615 | S | R | S | S | R | S | R | S | R | S | S | S |
| 16617 | S | R | S | R | S | S | R | R | R | S | R | R |
| 16618 | S | R | R | S | S | S | R | S | S | S | R | R |
| 16620 | S | R | R | S | R | R | R | R | S | S | S | R |
| 16621 | S | R | R | S | S | S | R | R | R | S | S | S |
| 16622 | S | R | S | R | R | R | R | S | S | R | S | R |
| 16623 | S | S | R | R | S | S | S | R | S | R | S | S |
| 16625 | S | R | S | S | S | S | S | R | S | R | R | S |
| 16626 | S | S | S | S | S | S | R | R | S | S | R | S |
| 16627 | S | R | S | S | S | S | S | R | R | R | R | S |
| 16629 | S | R | R | R | R | S | S | R | S | S | S | S |
| 16631 | S | R | R | R | S | R | R | S | S | R | S | S |
| 16633 | R | S | R | S | S | R | R | S | R | R | S | S |
| 16635 | R | S | S | R | S | R | S | R | R | R | R | R |
| 16636 | S | R | R | R | S | R | R | R | R | S | R | S |
| 16639 | S | S | S | R | R | R | S | S | R | S | S | R |
| 16641 | S | S | S | S | R | R | R | S | R | R | R | R |
| 16645 | S | S | R | S | R | R | S | S | S | R | R | R |
| 16648 | S | S | S | S | R | R | R | S | S | S | S | S |
| 16649 | S | S | S | R | S | S | R | S | R | S | R | S |
| 16650 | S | R | R | S | R | S | R | R | S | R | S | R |
| 16652 | S | S | S | S | S | R | R | R | S | S | R | R |
| 16655 | S | R | R | S | S | S | R | R | R | S | R | S |
| 16657 | S | R | S | R | S | R | S | S | R | S | R | S |
| 16658 | S | S | S | R | R | S | S | S | R | S | R | S |
| 16660 | S | S | R | R | R | R | R | R | R | R | S | S |
| 16663 | S | R | S | R | S | S | S | R | R | S | S | S |
| 16666 | S | R | S | R | R | R | S | R | R | S | R | R |
| 16667 | S | R | S | S | R | S | S | S | R | R | S | R |
| 16668 | S | R | R | S | R | S | R | R | S | S | S | S |
| 16669 | S | R | S | R | R | R | S | R | S | S | S | S |
| 16671 | S | S | S | R | R | R | S | R | R | R | S | S |
| 16673 | R | S | S | S | R | R | R | S | R | S | S | S |
| 16674 | S | S | S | S | S | R | S | S | S | S | S | S |
| 16675 | S | S | R | R | R | S | S | R | R | R | S | R |
| 16676 | S | S | S | S | R | S | S | R | R | R | S | R |
| 16677 | S | R | S | S | R | S | S | R | R | S | S | R |
| 16683 | S | S | R | R | R | S | S | S | S | R | R | S |
| 16684 | S | R | R | R | S | S | S | R | R | R | S | S |
| 16685 | S | R | R | S | S | R | S | S | R | R | S | R |
| 16687 | S | R | S | R | R | S | S | S | R | R | R | R |
| 16688 | S | R | R | R | S | S | R | R | R | S | S | S |
| 16692 | R | S | S | S | R | R | R | R | R | S | S | R |
| 16693 | S | S | S | S | R | S | S | R | S | S | R | R |
| 16694 | S | S | R | S | R | S | R | S | S | R | R | S |
| 16697 | S | R | S | R | R | R | S | S | S | S | S | R |
| 16699 | S | R | S | R | S | S | R | S | S | S | R | S |
| 16701 | S | S | S | S | R | S | R | R | R | R | R | S |
| 16702 | S | S | S | S | R | R | R | S | R | S | S | R |
| 16704 | S | R | R | R | R | R | S | R | R | S | S | R |
| 16709 | S | S | R | S | S | R | S | R | S | R | S | S |
| 17298 | | | R | | | R | R | | R | | | |
| 17299 | | | S | | | S | S | | S | | | |
| 17300 | | | R | | | S | S | | R | | | |
| 17301 | | | S | | | R | R | | S | | | |

| | |
|---|---|
| 3833 | Chirality mix |
| 3833 | Chirality mix* |
| 18946 | PO in the gap* |
| 18947 | Full PO* |

All of the compounds were assessed in a single experiment except where marked * when a separate experiment was performed

### Experimental

### LNA Oligonucleotide Mediated Cleavage of RNA by RNase H1(Recombinant Human).

LNA oligonucleotide 15 pmol and 5″fam labeled RNA 45 pmol was added to 13 μL of water. Annealing buffer 6 μL (200 mM KCl, 2 mM EDTA, pH 7.5) was added and the temperature was raised to 90° C. for 2 min. The sample was allowed to reach room temperature and added RNase H enzyme (0.15 U) in 3 μL of 750 mM KCl, 500 mM Tris-HCl, 30 mM MgCl₂, 100 mM dithiothreitol, pH 8.3). The sample was kept at 37° C. for 30 min and the reaction was stopped by adding EDTA solution 4 μL (0.25 M).

### AIE-HPLC of Cleaved RNA Samples

The sample 15 μL was added to 200 μL of buffer A (10 mM NaClO4, 1 mM EDTA, 20 mM TRIS-HCL pH 7.8). The sample was subjected to AIE-HPLC injection volume 50 μL(Collumn DNA pac 100 2×250, gradient 0 min. 0.25 mL/min. 100% A, 22 min. 22% B(1 mM NaClO4, 1 mM EDTA, 20 mM TRIS-HCL pH 7.8), 25 min. 0.25 mL/min. 100% B, 30 min. 0.25 mL/min. 100% B, 31 min. 0.5 mL/min. 0% B, 35 min. 0.25 mL/min. 0% B, 40 min. 0.25 mL/min. 0% B. Signal detention fluorescens emission at 518 nm exitation at 494 nm.

### Results

LNA oligonucleotide with the sequence G^{m}CattggtatT^{m}CA all phosphorus linkages thiolated. The specific chirality of the thiophosphate in the linkages are noted. Where nothing are noted the chirality are a mix of R and S. Under the AIE-HPLC retention time the percentage's the peaks areas of the sum the all peak areas are listed. The ranking number of the activity of the different LNA-oligonucleotides are calculated from the % of full length RNA left after the enzyme reaction the chirality mixed LNA oligonucleotide 3833 divided with what was left of the RNA for the other LNA oligonucleotides.

| | | | | | | | Full | Full length |
|---|---|---|---|---|---|---|---|---|
| | | | | | | | length | 3833/chiral |
| Oligo | AIE HPLC retention time (% of total) | | | | | | % | full length |
| no. | 11.05 | 11.367 | 11.742 | 12.3 | 12.75 | 12.942 | 15.017 | 3833 |
| 16614 | 1.9 | 19.3 | 3.5 | 63.4 | 0.0 | 0.0 | 11.9 | 4.4 |
| 16615 | | | | | | | | |
| 16617 | 0.7 | 18.6 | 4.1 | 44.4 | 5.8 | 7.0 | 19.5 | 2.7 |
| 16618 | 2.2 | 16.1 | 6.1 | 45.9 | 5.1 | 8.1 | 16.5 | 3.2 |
| 16620 | 1.1 | 8.8 | 14.5 | 26.4 | 4.0 | 31.4 | 13.9 | 3.8 |
| 16621 | 2.2 | 1.8 | 32.9 | 37.4 | 0.0 | 11.5 | 14.2 | 3.7 |
| 16622 | 2.3 | 57.1 | 15.5 | 16.7 | 1.6 | 2.1 | 4.7 | 11.2 |
| 16623 | 2.8 | 3.7 | 22.9 | 60.7 | 1.7 | 3.6 | 4.7 | 11.2 |
| 16625 | 2.7 | 3.2 | 20.7 | 28.9 | 2.8 | 20.6 | 21.1 | 2.5 |
| 16626 | 1.3 | 3.2 | 4.6 | 34.0 | 6.0 | 30.1 | 20.9 | 2.5 |
| 16627 | 1.8 | 3.8 | 26.4 | 19.0 | 4.2 | 29.8 | 15.0 | 3.5 |
| 16629 | 1.7 | 2.4 | 36.3 | 38.6 | 2.6 | 5.7 | 12.8 | 4.1 |
| 16631 | 2.6 | 55.3 | 7.8 | 6.5 | 14.9 | 3.8 | 9.2 | 5.7 |
| 16633 | 0.0 | 50.3 | 7.1 | 4.8 | 6.4 | 18.8 | 12.5 | 4.2 |
| 16635 | 1.8 | 7.2 | 64.9 | 7.1 | 11.1 | 4.0 | 3.9 | 13.5 |
| 16636 | 2.1 | 3.8 | 8.9 | 6.4 | 27.9 | 11.6 | 39.3 | 1.3 |
| 16639 | 3.8 | 17.9 | 71.3 | 5.3 | 0.0 | 0.0 | 1.7 | 30.6 |
| 16641 | 1.9 | 41.7 | 10.3 | 10.7 | 2.5 | 13.5 | 19.4 | 2.7 |
| 16645 | 2.2 | 14.1 | 39.8 | 8.6 | 0.0 | 19.2 | 16.0 | 3.3 |
| 16648 | 1.2 | 3.3 | 22.2 | 55.7 | 1.8 | 2.6 | 13.2 | 3.9 |
| 16649 | 2.4 | 37.4 | 3.7 | 28.2 | 7.6 | 0.0 | 20.8 | 2.5 |
| 16650 | 1.3 | 5.6 | 5.6 | 58.3 | 0.0 | 22.3 | 6.8 | 7.7 |
| 16652 | 2.8 | 3.3 | 10.4 | 5.1 | 9.7 | 43.0 | 25.8 | 2.0 |
| 16655 | 0.0 | 3.5 | 3.8 | 20.2 | 4.7 | 21.2 | 46.5 | 1.1 |
| 16657 | 0.0 | 12.2 | 73.4 | 3.5 | 7.8 | 0.0 | 3.1 | 16.8 |
| 16658 | 0.0 | 15.9 | 34.2 | 37.0 | 0.9 | 2.4 | 9.6 | 5.5 |
| 16660 | 0.0 | 0.0 | 0.0 | 0.0 | 0.0 | 0.0 | 100.0 | 0.5 |
| 16663 | 0.0 | 4.5 | 38.3 | 25.9 | 7.5 | 4.9 | 19.0 | 2.7 |
| 16666 | 0.0 | 2.0 | 76.0 | 3.7 | 0.0 | 0.0 | 18.3 | 2.9 |
| 16667 | 0.0 | 31.5 | 30.1 | 25.5 | 0.0 | 9.3 | 3.7 | 14.3 |
| 16668 | 0.0 | 4.7 | 4.7 | 61.3 | 0.0 | 21.9 | 7.5 | 7.0 |
| 16669 | 0.0 | 3.7 | 76.3 | 6.4 | 1.9 | 2.6 | 9.1 | 5.7 |
| 16671 | | | | | | | | |
| 16673 | 0.0 | 9.1 | 15.7 | 31.1 | 0.0 | 3.7 | 40.4 | 1.3 |
| 16674 | 0.0 | 0.0 | 0.0 | 7.8 | 0.0 | 0.0 | 92.2 | 0.6 |
| 16675 | 0.0 | 15.4 | 20.5 | 25.3 | 4.0 | 21.9 | 12.9 | 4.1 |
| 16676 | 0.0 | 1.6 | 29.2 | 33.1 | 0.0 | 17.2 | 18.9 | 2.8 |
| 16677 | 2.1 | 36.5 | 7.0 | 47.6 | 0.0 | 5.2 | 1.6 | 32.4 |
| 16683 | 1.5 | 17.8 | 34.3 | 20.2 | 2.8 | 2.3 | 20.9 | 2.5 |
| 16684 | 1.2 | 13.6 | 1.6 | 35.4 | 8.6 | 0.0 | 39.5 | 1.3 |
| 16685 | 0.0 | 3.4 | 78.6 | 7.4 | 2.1 | 2.2 | 6.3 | 8.3 |
| 16687 | 1.1 | 54.8 | 8.9 | 7.4 | 1.0 | 6.8 | 19.9 | 2.6 |
| 16688 | 1.1 | 16.8 | 55.1 | 3.4 | 6.3 | 12.1 | 5.2 | 10.1 |
| 16692 | 0.0 | 4.4 | 33.2 | 51.1 | 3.0 | 5.9 | 2.4 | 21.5 |
| 16693 | 0.0 | 4.4 | 30.4 | 28.7 | 0.0 | 28.9 | 7.6 | 6.8 |
| 16694 | 0.0 | 5.2 | 37.6 | 20.8 | 2.2 | 17.3 | 16.9 | 3.1 |
| 16697 | | | | | | | | |
| 16699 | 1.5 | 1.6 | 17.5 | 19.3 | 6.8 | 9.0 | 44.3 | 1.2 |
| 16701 | 0.0 | 4.2 | 6.4 | 44.2 | 0.0 | 29.4 | 15.7 | 3.3 |
| 16702 | 0.0 | 27.3 | 20.4 | 22.9 | 1.2 | 8.7 | 19.5 | 2.7 |
| 16704 | 0.0 | 3.2 | 52.4 | 3.3 | 1.4 | 2.8 | 36.9 | 1.4 |
| 16709 | 8.5 | 31.4 | 4.1 | 2.3 | 8.7 | 25.0 | 20.0 | 2.6 |
| 17298 | 0.0 | 12.0 | 25.1 | 44.4 | 4.1 | 11.7 | 2.7 | 19.2 |
| 17299 | | | | | | | | |
| 17300 | | | | | | | | |
| 17301 | 1.8 | 17.2 | 2.7 | 4.7 | 8.9 | 16.2 | 48.5 | 1.1 |
| 3833 | 1.0 | 6.8 | 13.7 | 11.1 | 3.9 | 11.2 | 52.3 | 1.0 |
| 3833 | | | | | | | 10 | 1 |
| 18946 | 2.4 | 8.3 | 29.9 | 18.3 | 10.9 | 10.6 | 19.6 | 0.5 |
| 18947 | 0.0 | 8.8 | 34.0 | 21.6 | 10.5 | 10.5 | 14.6 | 0.7 |

### Conclusion

The chirality of the phosphorothioate linkages of the LNA oligonucleotide are randomly chosen except for the last 5″coupling where the S chirality were selected and the LNA oligonucleotides where spot chirality was chosen 17298-17301. The full diester and diester only in the gap version of the LNA oligonucleotide have less activity than the mixed chiral version 3833. The chiral sequence enhances the activation and cleavage of the RNA. For most of the specific chiral LNA oligonucleotides the activation of RNaseH1 worked better than for the chirality mixed 3833. The best of the specific sequences initiated substantial more cleavage of RNA than 3833 (98.4% versus 47.7% after 30 minutes). A characteristic of each of the specific LNA oligonucleotides are their unique cleavage pattern of the RNA varying form one to several cleavage points.

### Example 8 In Vitro Toxicity Screening in Primary Hepatocytes

### Mouse Liver Perfusion

Primary mouse hepatocytes were isolated from 10- to 13-week old male C57Bl6 mice by a retrograde two-step collagenase liver perfusion. Briefly, fed mice were anaesthetized with sodium pentobarbital (120 mg/kg, i.p.). Perfusion tubing was inserted via the right ventricle into the v. cava caudalis. Following ligation of the v. cava caudalis distal to the v. iliaca communis, the portal vein was cut and the two-step liver perfusion and cell isolation was performed. The liver was first perfused for 5 min with a pre-perfusing solution consisting of calcium-free, EGTA (0.5 mM)-supplemented, HEPES (20 mM)-buffered Hank's balanced salt solution, followed by a 12-min perfusion with NaHCO3 (25 mM)-supplemented Hank's solution containing CaCl2 (5 mM) and collagenase (0.2 U/ml; Collagenase Type II, Worthington). Flow rate was maintained at 7 ml/min and all solutions were kept at 37° C. After in situ perfusion, the liver was excised, the liver capsule was mechanically opened, the cells were suspended in William's Medium E (WME) without phenol red (Sigma W-1878), and filtered through a set of nylon cell straines (40- and 70-mesh). Dead cells were removed by a Percoll (Sigma P-4937) centrifugation step (percoll density: 1.06 g/ml, 50 g, 10 min) and an additional centrifugation in WME (50×g, 3 min).

### Compounds Used

| | |
|---|---|
| 5′-^{m}CₓAₓ^{m}Cₓaₓtₓtₓcₓcₓtₓtₓgₓcₓtₓ^{m}CₓTₓG-3\grave{ } | (Parent #56) |
| 5′-^{m}CₓAₓ^{m}Cₓaₓtₓtₛcₓcₓtₓtₛgₓcₓtₛ^{m}CₓTₛG-3\grave{ } | (Comp #57) |
| 5′-^{m}CₓAₓ^{m}Cₓaₓtₓtᵣcₓcₓtₓtᵣgₓcₓtᵣ^{m}CₓTᵣG-3\grave{ } | (Comp #58) |
| 5′-^{m}CₓAₓ^{m}Cₓaₓtₓtₛcₛcₓtₓtₛgₛcₓtₓ^{m}CₓTₓG-3\grave{ } | (Comp #59) |
| 5′-^{m}CₓAₓ^{m}Cₓaₓtₓtᵣcᵣcₓtₓtᵣgᵣcₓtₓ^{m}CₓTₓG-3\grave{ } | (Comp #60) |

Capital letters are beta-D-oxy LNA nucleosides, small letters are DNA nucleosides

Subscript x=randomly incorporated phosphorothioate linkage from a racemic mixture of Rp and Sp monomers.

Subscript s=stereocontrolled phosphoramidite linkage from a Sp monomer

Subscript r=stereocontrolled phosphoramidite linkage from a Rp monomer

Superscript m preceding a capital C represents 5-methyl cytosine LNA nucleoside.

### Hepatocyte Culturing

For cell culture, primary mouse hepatocytes were suspended in WME supplemented with 10% fetal calf serum, penicillin (100 U/ml), streptomycin (0.1 mg/ml) at a density of approx. 5×10⁶ cells/ml and seeded into collagen-coated 96-well plates (Becton Dickinson AG, Allschwil, Switzerland) at a density of 0.25×10⁶ cells/well. Cells were pre-cultured for 3 to 4h allowing for attachment to cell culture plates before start of treatment with oligonucleotides. Oligonucleotides dissolved in PBS were added to the cell culture and left on the cells for 3 days. Cytotoxicity levels were determined by measuring the amount of Lactate dehydrogenase (LDH) released into the culture media using a Cytotoxicity Detection Kit (Roche 11644793001, Roche Diagnostics GmbH Roche Applied Science Mannheim, Germany) according to the manufacturer's protocol. For the determination of cellular ATP levels we used the CellTiter-Glo® Luminescent Cell Viability Assay (G9242, Promega Corporation, Madison Wis., USA) according to the manufacturer's protocol. Each sample was tested in triplicate.

### Target Knock-Down Analysis

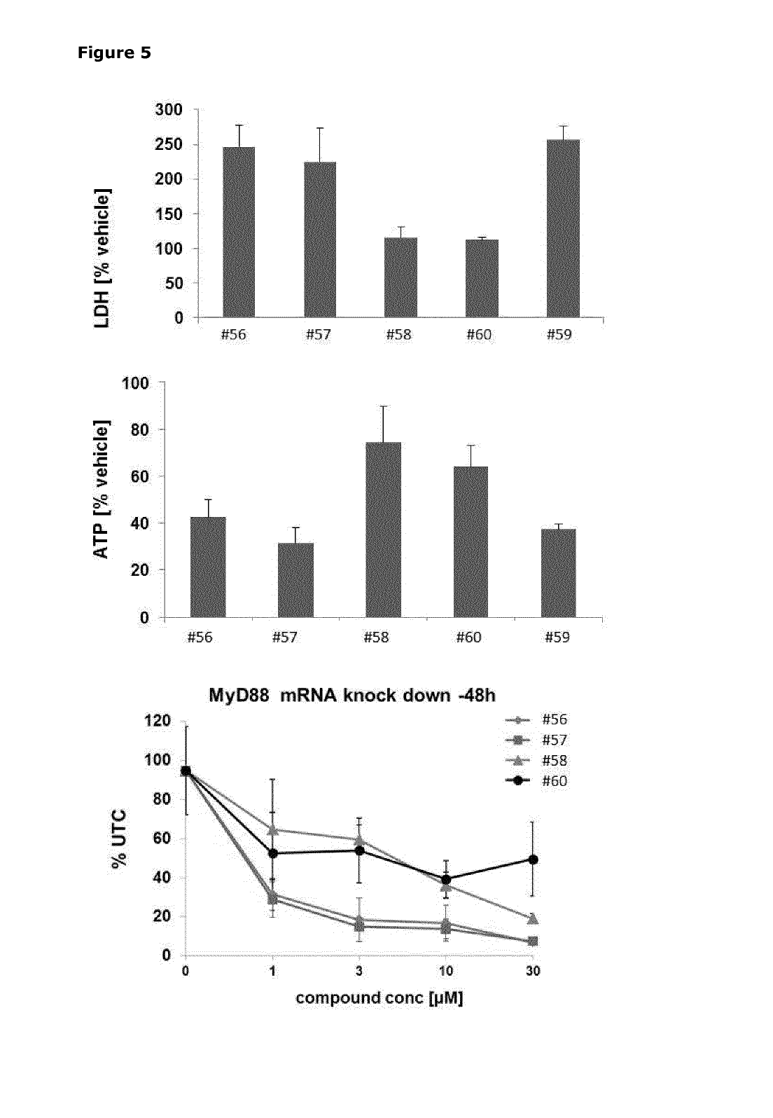

mRNA purification from mouse hepatocytes RNeasy 96 Kit (Qiagen, Hombrechtikon, Switzerland) including an RNAse free DNAse I treatment according to the manufacturer's instructions. cDNA was synthesized using iScript single strand cDNA Synthesis Kit (Bio-Rad Laboratories AG, Reinach, Switzerland). Quantitative real-time PCR assays (qRT-PCR) were performed using the Roche SYBR Green I PCR Kit and the Light Cycler 480 (Roche Diagnostics, Rotkreuz, Switzerland) with specific DNA primers. Analysis was done by the ΔCt threshold method to determine expression relative to RPS12 mRNA. Each analysis reaction was performed in duplicate, with two samples per condition. The results are shown in FIGS. 5 & 6. Compounds #58 and #60 have significantly reduced toxicity whilst retaining effective antisense activity against the target (Myd88). These compounds comprise Rp stereodefined phosphorothioate linkages.

### Example 9 Nephrotoxicity Screening Assay

The same compounds as used in example 6 and 8 were used in the following RPTEC-TERT1 culture, oligonucleotide treatment and viability assay:

RPTEC-TERT1 (Evercyte GmbH, Austria) were cultured according to the manufacturer's instructions in PTEC medium (DMEM/F12 containing 1% Pen/Strep, 10 mM Hepes, 5.0 μg/ml human insulin, 5.0 μg/ml human transferrin, 8.65 ng/ml sodium selenite, 0.1 μM hydrocortisone, 10 ng/ml human recombinant Epidermal Growth Factor, 3.5 μg/ml ascorbic acid, 25 ng/ml prostaglandin E1, 3.2 pg/ml Triiodo-L-thyronine and 100 μg/ml Geneticin). For viability assays, PTEC-TERT1 were seeded into 96-well plates (Falcon, 353219) at a density of 2×10⁴ cells/well in PTEC medium and grown until confluent prior to treatment with oligonucleotides. Oligonucleotides were dissolved in PBS and added to the cell culture at a final concentration of 10 or 30 μM. Medium was changed and oligonucleotides were added fresh every 3 days. After 9 days of oligonucleotide treatment, cell viability was determined by measurement of cellular ATP levels using the CellTiter-Glo® Luminescent Cell Viability Assay (G7571, Promega Corporation, Madison Wis., USA) according to the manufacturer's protocol. The average ATP concentration and standard deviation of triplicate wells were calculated. PBS served as vehicle control.

The results are shown in FIG. 8. Compound #10 shows reduced nephrotoxicity as compared to the non-stereospecified compound #1 and compound #14. Stereospecified compounds #57, #58, #60 show significantly reduced nephrotoxicity as compared to the parent compound (#56).

### Example 10 Mismatch Specificity of Chirally Defined Phosphorothioate LNA Gapmers

The experimental procedure used was as described in example 7, with the exception that alternative RNA substrates were used which introduced a mismatch at various positions as compared to the parent 3833 compound. The RNaseH activity against the perfect match RNA substrate and the mismatch RNA substrates was determined.

TABLE 3
Effect of mismatches on RNaseH activity of 3833.

| RNA: SEQ | | | Tm | % full |
|---|---|---|---|---|
| ID | RNA Substrate | TM up | down | length |
| 5 | ACAGAAUACCAAUGCACAGA | 59.5 | 59.4 | 39.1 |
| 6 | UGAGAAUACCAAUGCUAAGU | 57.8 | 59.8 | |
| 7 | CAGGAAUACCAAUGCAGAGA | 59.2 | 61.8 | 58.3 |
| 8 | AGUGGAUACCAAUGCUGCAG | 53.4 | 55.7 | 54.6 |
| 9 | UUUGGAUACCAAUGCAUAGG | 54.1 | 57.1 | 60.7 |
| 10 | UCUGAGUACCAAUGCCAUGA | 55.0 | 55.5 | 43.7 |
| 11 | GCUGAAUGCCAAUGCUGAGU | 56.9 | 57.6 | 67.4 |
| 12 | UCUGAAUACCGAUGCUUUAA | 57.3 | 58.0 | 42.8 |
| 13 | UCUGAAUACCAGUGCUUUAA | 56.0 | 57.7 | 43.9 |
| 14 | CUUGUAAUACCAAUGCUAUAA | 51.9 | 52.5 | 48.5 |
| 15 | AAAGAAUACCAAUGUUCUCU | 49.2 | 49.8 | |
| 16 | UAUGAAUACCAUUGUCUUAU | 40.5 | 41.4 | 72.0 |
| 17 | CCGAAUGCCAAUGCAGAGUU | 57.1 | 58.0 | 75.2 |
| 18 | GAUGAAAUACCAAUGUUAACU | 39.6 | 40.8 | |
| 19 | CUGAAUACCAAUGCUGAACUU | 59.0 | 59.9 | 49.9 |

Mismatches are shown by use of a larger font size. RNaseH cleavage analysed after 30 minutes. The cleavage products changes with the position of the mismatch.

TABLE 4
Effect of mismatches on RNaseH activity of stereodefined variants
of 3833.

| | | | | Relative | Relative |
|---|---|---|---|---|---|
| SEQ | | | % | activity | activity |
| ID | | | Full | of mis- | of full |
| NO | RNA Substrate | LNA | Length | match | match |
| 9 | UUUGGAUACCAAUGCAUAGG | 3833 | 37.7 | 1 | 1 |
| | | 16639 | 25.5 | 1.5 | 30.6 |
| | | 16657 | 7.9 | 4.8 | 16.8 |
| | | 16685 | 32.8 | 1.2 | 8.3 |
| 12 | UCUGAAUACCGAUGCUUUAA | 3833 | 53.0 | 1 | 1 |
| | | 16650 | 71.7 | 0.7 | 7.7 |
| | | 16668 | 79.5 | 0.7 | 7.0 |
| 13 | UCUGAAUACCAGUGCUUUAA | 3833 | 46.4 | 1 | 1 |
| | | 16635 | 8.5 | 5.4 | 13.5 |
| | | 16639 | 2.6 | 18.0 | 30.6 |
| | | 16657 | 28.3 | 1.6 | 16.8 |
| | | 16685 | 33.8 | 1.4 | 8.3 |

To a perfect match RNA substrate, chirally defined phosphorothioate oligonucleotides tend to activate RNaseH mediated cleavage of RNA more profound than the ASO with mixed chirality. However, chirally defined oligonucleotides of a chosen phosphorothioate (ASO) configuration can be found that have a marked reduced RNaseH cleavage of a mismatch

RNA, highlighting the ability to screen libraries of chirally defined variants of an oligonucleotide to identify individual stereodefined compounds which have improved mismatch selectivity.

### Example 11

The parent compound used, 4358 was used:

5″Gₛ^{m}CₛaₛaₛgₛcₛaₛtₛcₛcₛtₛGₛT 3′ (SEQ ID NO 5)

Wherein capital letters represent beta-D-oxy-LNA nucleosides, lower case letters represent DNA nucleosides, subscript s represents random s or r phosphorothioate linkages (not chirally defined during oligonucleotide synthesis), and superscript m prior to C represents 5-methyl cytosine LNA nucleoside.

A range of fully chirally defined variants of 4358 were designed with unique patterns of R and S at each of the 11 internucleoside positions, as illustrated by either an S or an R. The RNaseH recruitment activity and cleavage pattern was determined using human RNase H, and compared to the parent compound 4358 (chirality mix). The results obtained were as follows:

| | | | | | | | | | | | | | Full length |
|---|---|---|---|---|---|---|---|---|---|---|---|---|---|
| Oligo | Chirality of nucleobase linkages | | | | | | | | | | | % full | 4358/full |
| no. | 1 | 2 | 3 | 4 | 5 | 6 | 7 | 8 | 9 | 10 | 11 | length | length chiral |
| 4358 | Chirality mix | | | | | | | | | | | 4.34 | 1.0 |
| 24387 | S | S | S | S | S | S | S | S | S | S | S | 4.30 | 1.01 |
| 24388 | S | S | S | S | S | S | R | S | S | S | S | 2.64 | 1.64 |
| 24389 | S | S | S | R | S | S | S | S | S | S | S | 4.01 | 1.08 |
| 24390 | S | S | R | S | S | S | S | S | S | S | S | 4.14 | 1.05 |

1. A stereodefined phosphorothioate LNA oligonucleotide comprising at least one stereodefined phosphorothioate linkage between a LNA nucleoside and a subsequent (3′) nucleoside.

2. The stereodefined phosphorothioate LNA oligonucleotide of claim 1, which comprises at least one stereospecific phosphorothioate nucleotide pair wherein the internucleoside linkage between the nucleosides of the at least one stereospecific phosphorothioate nucleotide pair is either in the Sp configuration or in the Rp configuration, and wherein at least one of the nucleosides of the at least one stereospecific phosphorothioate nucleotide nucleoside pair is a LNA nucleoside.

3. The stereodefined phosphorothioate LNA oligonucleotide of claim 2, wherein the other nucleoside of the at least one stereospecific phosphorothioate nucleotide nucleotide pair is other than DNA, such as nucleoside analogue, such as a further LNA nucleoside or a 2′ substituted nucleoside.

4. The stereodefined phosphorothioate LNA oligonucleotide of claim 1, wherein the LNA oligonucleotide is a gapmer oligonucleotide.

5. The stereodefined phosphorothioate LNA oligonucleotide of claim 4, wherein the phosphorothioate internucleoside linkage between at least two adjacent LNA nucleosides is stereospecific, Sp or Rp.

6. The stereodefined phosphorothioate LNA oligonucleotide of claim 4, wherein each wing of the gapmer comprises one or more stereospecific phosphorothioate internucleoside linkage between at least two adjacent LNA nucleosides.

7. The stereodefined phosphorothioate LNA oligonucleotide of claim 4, wherein all the phosphorothioate internucleoside linkages between adjacent LNA nucleosides are stereospecific.

8. The stereodefined phosphorothioate LNA oligonucleotide of claim 4, wherein the oligonucleotide comprises a region Y′ which is capable of recruiting RNase H, which is flanked 5′ and 3′ by 1-6 nucleotide analogue units.

9. The stereodefined phosphorothioate LNA oligonucleotide of claim 8, wherein the nucleoside analogue units are independently selected from the group consisting of 2′-O-methoxyethyl-RNA (2′MOE), 2′-fluoro-DNA monomers or LNA nucleoside analogues.

10. The stereodefined phosphorothioate LNA oligonucleotide of claim 9, wherein the nucleoside analogue units are LNA units.

11. The stereodefined phosphorothioate LNA oligonucleotide of claim 1, wherein the LNA units are selected from the group consisting of (R)-cET, and (S)-cET.

12. The stereodefined phosphorothioate LNA oligonucleotide of claim 1, wherein the LNA units are beta-D-oxy LNA units.

13. A conjugate comprising the stereodefined phosphorothioate LNA oligonucleotide of claim 1 covalently attached to a non-nucleoside moiety.

14. A pharmaceutical composition comprising the stereodefined phosphorothioate LNA oligonucleotide of claim 1 or the conjugate of claim 13 and a pharmaceutically acceptable solvent, diluent, carrier, salt or adjuvant.

15. The stereodefined phosphorothioate LNA oligonucleotide of claim 1 or the conjugate of claim 13, for use in medicine.


SEQUENCE LISTING (5 sequences)

SEQ ID NO: 1 - DNA, 16 bp, artificial
  Oligonucleotide sequence
actgctttccactctg

SEQ ID NO: 2 - DNA, 14 bp, Artificial
  Oligonucleotide sequence
tcatggctgcagct

SEQ ID NO: 3 - DNA, 13 bp, Artificial
  Oligonucleotide sequence
gcattggtattca

SEQ ID NO: 4 - DNA, 16 bp, Artificial
  Oligonucleotide sequence
cacattccttgctctg

SEQ ID NO: 5 - DNA, 13 bp, Artificial
  Oligonucleotide sequence
gcaagcatcctgt